(19) United States
(12) Patent Application Publication
(10) Pub. No.: US 2023/0268041 A1
(43) Pub. Date: Aug. 24, 2023

(54) SYSTEM AND METHOD FOR USING THE MICROBIOME TO IMPROVE HEALTHCARE

(71) Applicant: Jona, Inc., Darien, CT (US)

(72) Inventor: Leo GRADY, Darien, CT (US)

(73) Assignee: Jona, Inc., Darien, CT (US)

(21) Appl. No.: 18/172,878

(22) Filed: Feb. 22, 2023

Related U.S. Application Data
(60) Provisional application No. 63/268,380, filed on Feb. 23, 2022.
(60) Provisional application No. 63/269,842, filed on Mar. 24, 2022.
(60) Provisional application No. 63/482,257, filed on Jan. 30, 2023.

(51) Int. Cl. G16H 15/00

(52) U.S. Cl. CPC G16H 15/00

(57) ABSTRACT

Systems and methods for leveraging subject microbiome data to achieve a target goal are disclosed. The method contains operations including: detecting, on a graphical user interface of an application platform of a user computing device, a selection of a subject by a user; accessing, based on the detecting, data associated with the subject, wherein the data comprises the subject microbiome data; generating, by a processor, an overview report comprising a first set of subject predispositions; and displaying, on the application platform, the generated overview report. Other aspects are described and claimed.

### CROSS REFERENCE TO RELATED APPLICATIONS

This application claims priority to U.S. Application No. 63/268,380, filed on Feb. 23, 2022; U.S. Application No. 63/269,842, filed on Mar. 24, 2022; and U.S. Application No. 63/482,257, filed on Jan. 30, 2023; all of which are incorporated by reference herein in their entireties.

### TECHNICAL FIELD

Various embodiments of the present disclosure relate generally to the field of healthcare improvement, and, more particularly, to data collection and analysis of the microbiome and the dynamic utilization of that analyzed data to impact diagnosis, treatment, and/or wellness.

### BACKGROUND

The human and animal microbiome has been demonstrated to play a key role in both human and animal health. Despite having been shown that the microbiome can affect early development, immune response, therapy efficacy and even mental and behavioral changes, the role of the microbiome is still poorly understood. One reason why the microbiome is so poorly understood is the lack of frequent, convenient and location-specific microbiome sampling across a population as well as the lack of information about the microbiome at the levels of interacting pairs or groups of individuals. The present disclosure is intended to address this problem by enabling more detailed and widespread data collection about the microbiome as well as the use of that data to impact diagnosis, treatment and wellness.

The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

In summary, a computer-implemented method of leveraging subject microbiome data is disclosed. The computer-implemented method contains operations including: detecting, on a graphical user interface of an application platform of a user computing device, a selection of a subject by a user; accessing, based on the detecting, data associated with the subject, wherein the data comprises the subject microbiome data; generating, by a processor, an overview report comprising a first set of subject predispositions; and displaying, on the application platform, the generated overview report.

Another aspect provides a user computing device for leveraging subject microbiome data. The user computing device includes: one or more computer processors; and a non-transitory computer-readable storage medium storing instructions executable by the one or more computer processors, the instructions when executed by the one or more computer processors causing the one or more computer processors to perform operations including: detecting, on a graphical user interface of an application platform associated with the user computing device, a selection of a subject by a user; accessing, based on the detecting, data associated with the subject, wherein the data comprises the subject microbiome data; generating, by a processor, an overview report comprising a first set of subject predispositions; and displaying, on the application platform, the generated overview report.

A further aspect provides a non-transitory computer-readable medium storing instructions executable by one or more computer processors of a computer system. The instructions, when executed by the one or more computer processors, cause the one or more computer processors to perform operations including: detecting, on a graphical user interface of an application platform of a user computing device, a selection of a subject by a user; accessing, based on the detecting, data associated with the subject, wherein the data comprises the subject microbiome data; generating, by a processor, an overview report comprising a first set of subject predispositions; and displaying, on the application platform, the generated overview report.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE DRAWINGS

The accompanying drawings, which are incorporated in and constitute a part of this specification, illustrate several embodiments and together with the description, serve to explain the principles of the disclosure.

FIG. 1 depicts a block diagram of an exemplary system environment for improving healthcare, according to one aspect of the present disclosure.

FIG. 2 depicts a block diagram of exemplary associations and linkages between entities associated with a health optimization system, according to one aspect of the present disclosure.

FIG. 3 depicts an exemplary flow diagram for training and deploying a machine learning model, according to one aspects of the present disclosure.

FIG. 4 depicts an exemplary knowledge graph, according to one aspect of the present disclosure.

FIG. 5 depicts an exemplary user interface, according to one aspect of the present disclosure.

FIG. 6 depicts an exemplary user interface, according to one aspect of the present disclosure.

FIG. 7 depicts an exemplary user interface, according to one aspect of the present disclosure.

FIG. 8 depicts an exemplary user interface, according to one aspect of the present disclosure

It is to be understood that both the foregoing general description and the following detailed description are exemplary and explanatory only and are not restrictive of the disclosed embodiments, as claimed.

### DETAILED DESCRIPTION OF THE EMBODIMENTS

The following embodiments describe systems and methods for using the microbiome to improve healthcare. In the context of this application, the term “microbiome” may refer to the one or more portions of the entire aggregate of all microbiota (including all related microbiota biological properties such as genetics, proteins, metabolites, transcriptomics, etc.) and properties of the environment that they reside on or within tissues and biofluids along with the corresponding anatomical sites in which they reside, including the skin, mammary glands, seminal fluid, uterus, placenta, ovarian follicles, lung, saliva, oral mucosa, nasal mucosa, conjunctiva, biliary tract, and gastrointestinal (GI) tract. For clarity, note that the anatomical site may be represented at one or more levels of specificity (“GI tract”, “duodenum”, “upper duodenum”, etc.). For clarity, note that the anatomical sites may vary from subject to subject (e.g., for plant versus animal) and note that the subject may be living or dead (necrobiome). Types of microbiota in our definition include bacteria, archaea, fungi, protists, viruses, phages, plasmids, prions, parasites, mobile genetic elements and micro-animals. The term “subject” is used herein throughout to refer to either humans, animals or plants since the inventive concepts described herein equally apply to both humans, animals and plants. Note that a subject may be sampled directly and/or may be linked to one or more subjects. Any references to “user” refers to either a subject themselves or to a person who has access to the subject's account/data (e.g., a user could be the subject themselves, a user could be a care or medical provider for the subject, a user could be a parent for the subject, etc.). One or more users may be associated with one or more subjects (e.g., a farm owner may be a “user” for multiple animal “subjects” on the farm, both a patient and their doctor may be a “user” for a single patient “subject”, etc.).

### I. Overview

At a high level, the embodiments described herein have a variety of different possible components and forms. One aspect includes a subject prediction engine, which may take multiple forms and will be the subject of subsequent sections. Described herein are a plurality of non-limiting, high level characteristics of steps involved in the subject prediction engine.

### A. Development (Training)

The system of the embodiments may optionally receive one or more elements of subject data (e.g., context factors, microbiome data, linkages, linked user data, etc.) for one or more subjects and store these measurements in an accessible electronic storage device. The system may further receive one or more measurements of microbiome data for one or more subjects (e.g., at one or more locations of each subject) and store these measurements in the same or different accessible electronic storage device. The system may optionally link these measurements (e.g., using a processor) to either one or more time points of subject measurements and/or measurements of one or more additional subjects and then store these measurements in the same or different electronic storage device. The system may further receive one or more target subject predispositions for one or more subjects and thereafter train or develop (e.g., using a processor) a subject prediction engine that can read these measurements and, if available, other subject data (e.g., context factors, microbiome data, linked subject data, linked user data, etc.) to predict the one or more target subject predispositions. This subject prediction engine can be represented in many different forms such as by storing the system representation on an electronic storage device, in electric memory, distributed across a network, as hardware (e.g., field programmable gate array (FPGA), etc.), and the like.

### B. Deployment

The system of the embodiments may optionally receive one or more elements of subject data (e.g., context factors, microbiome data, linkages, linked user data, etc.) for a subject and store these measurements in an accessible electronic storage device. The system may further receive one or more measurements of microbiome data for a subject (e.g., at one or more locations of each subject) and store these measurements in the same or different accessible electronic storage device. The system may optionally link these measurements (e.g., using a processor) to either one or more time points of subject measurements and/or measurements of one or more additional subjects and then store these measurements in the same or different electronic storage device. The system may then apply the subject prediction engine to determine one or more subject predispositions about the subject. These determined subject predispositions may be stored one or more electronic storage devices. Additionally or alternatively, the system may output the determined subject predispositions to one or more display devices. The system may optionally provide a user and/or coach with a series of visualizations, recommendations, guidances, interactive capabilities and services using an electronic storage device, visual display, etc.

In the following sections, various embodiments associated with different types of entities, linkages, sampling, data, analysis, and guidances, as well as different example implementations of these embodiments, are more thoroughly described.

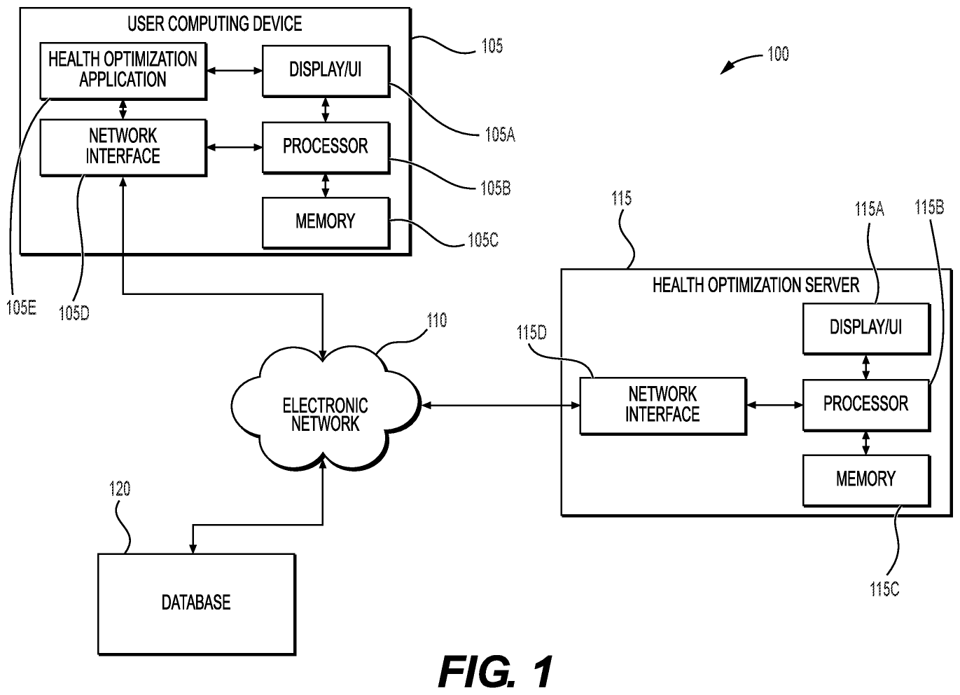

Referring now to FIG. 1, a block diagram depicting an exemplary system environment 100 for improving healthcare is provided. The system environment 100 may include a computing device 105 operated by a user, an electronic network 110, a health optimization server (“computer server”) 115, and a database 120. Each of the foregoing components may be connected via the electronic network 110, e.g., using one or more standard wired, data transfer and/or wireless communication protocols, and/or other means known to those skilled in the art but not explicitly listed here. The system environment 100, such as computer server 115, may include one or more computing devices. If the one or more processors of the computer system 100 are implemented as a plurality of processors, the plurality of processors may be included in a single computing device or distributed among a plurality of computing devices. If a computer server 115 comprises a plurality of computing devices, the memory of the computer server 115 may include the respective memory of each computing device of the plurality of computing devices. The computer server 115 and the database 120 may be one server computer device and a single database, respectively. Alternatively, the computer server 115 may be a server cluster, or any other collection or network of a plurality of computer servers. The database 120 also may be a collection of a plurality of interconnected databases. The computer server 115 and the database 120 may be components of one server system 100. Additionally, or alternatively, the computer server 115 and the database 120 may be components of different server systems, with the electronic network 110 serving as the communication channel between them.

The computing device 105 may include a display/user interface (UI) 105A, a processor 1056, a memory 105C, a network interface 105D, and/or a health optimization application (“application”) 105E. The user computing device 105 may be a personal computer (PC), a tablet PC, a television (TV), a smart TV a personal digital assistant (PDA), a mobile device, a palmtop computer, a laptop computer, a desktop computer, etc. The user computing device 105 may execute, by the processor 1056, an operating system (O/S) and at least one application (each stored in memory 105C). The application 105E may be a browser program or a mobile application program (which may also be a browser program in a mobile O/S). Users may be able to provide inputs to and receive outputs from the application 105E via interaction with one or more digital icons resident thereon. In some embodiments, outputs provided by the application 105E may be facilitated based on instructions/information stored in the memory 105C. The output may be visual data presented on the application GUI and may be executed, for instance, based on XML and Android programming languages or Objective-C/Swift. However, one skilled in the art would recognize that this may also be accomplished by other methods, such as webpages executed based on HTML, CSS, and/or scripts, such as JavaScript. The display/UI 105A may be a touch screen or a display with other input systems (e.g., mouse, keyboard, etc.). The network interface 105D may be a TCP/IP network interface for, e.g., Ethernet or wireless communications with the network 110. The processor 105B, while executing the application 105E, may receive user inputs from the display/UI 105A, and perform actions or functions in accordance with the application or other related applications.

The computer server 115 may include a display/UI 115A, a processor 1158, a memory 115C, and/or a network interface 115D. The computer server 115 may be a computer, system of computers (e.g., rack server(s)), and/or or a cloud service computer system. The computer server 115 may execute, by the processor 1158, an operating system (O/S) and at least one instance of a server program (each stored in memory 115C). The computer server 115 may store or have access to information from the database 120. The display/UI 115A may be a touch screen or a display with other input systems (e.g., mouse, keyboard, etc.) for an operator of the computer server 115 to control the functions of the computer server 115 (e.g., update the server program and/or the server information). The network interface 115D may be a TCP/IP network interface for, e.g., Ethernet or wireless communications with the network 110.

The computer server 115 may be configured to receive data over the network 110 from the user computing device(s) 105, including, but not limited to: subject data, user data, coach data, and user command requests. Subject, user, and/or coach data may be stored in the database 120 and may include previously acquired data (e.g., from a subject, user, coach or other sources) such as, for instance, contextual information associated with one or more subjects and/or objective sample measurements associated with one or more subjects.

### II. Entities

Three different types of entities are the primary focus of the concepts described herein: subjects, users and coaches.

### A. Subjects

Subjects correspond to those entities for which the system of the embodiments is gathering context data, microbiome data, personalized modeling, generating predispositions and providing guidance of suggested changes to achieve a goal. Subjects may represent a variety of different entities, including but not limited to: individual consumers, patients, enrollees in clinical trials, pets, livestock, zoo animals, plants, etc.

### B. Users

Although subjects are a primary focus of the concepts described herein, a subject may or may not always be a user of a system. Although the simplest scenario is where a subject and a user are the same person, there are many other scenarios in which this is not the case. For example, if the subject is a patient, the user of the system might be a doctor or a researcher. In another example, the subject of the system could be a pet and the user could be the pet owner. Therefore, users may represent a variety of different individuals, including but not limited to an individual consumer, family member (e.g., parent, caregiver, etc.), doctor, nutritionist, researcher, economist, epidemiologist, therapist, pet owner, farm owner, veterinarian, etc. Users may be assumed to have an interest in one or more subjects (or populations of subjects) and may or may not set goals for these subjects to achieve.

For clarity, different users may have different levels of access to the same subject. For example, a user who is themselves a subject may have access to all the subject data. However, a doctor user for the subject may have access to less subject information (e.g., restricted to purely medical information) and a researcher may have access to a very limited set of anonymized data over a population of subjects. Subject data access levels may be established for different users via interactions with the application 105E. These access levels may be established and/or adjusted manually (e.g., via selections made in a settings menu, etc.) or, alternatively, may established automatically (e.g., a researcher user that registers with the application 105E may automatically be allocated limited subject data access permissions, etc.).

Note that some implementations of the system may not distinguish users from subjects and just assume that each user corresponds to a subject. In such an implementation of the inventive concepts, any reference to a user would simply be assumed to refer to the subject themselves.

### C. Coaches

A third entity contemplated herein is a coach, which is entirely optional in an implementation of the system. Coaches are intended to provide support to users to help the users achieve goals, answer questions, etc. Therefore, coaches may represent a variety of different individuals, including but not limited to fitness coaches, diet coaches, pet coaches, agricultural coaches, nutritionists, doctors, therapists, and the like.

### III. Linkages

Subjects, users, and coaches are linked both within these groups and between each other to represent a variety of relationships and to substantially enhance the information and actionability produced by the inventive concepts. Contemplated herein are several different types of linkage that are referred to in a general sense as “linkages”. When important to specify which type of linkage is referred to, a modifier for the linkage type (e.g., coach-user linkage, subject-subject linkage, etc.) will be used.

Note that user-user, coach-coach, or coach-subject linkages are not further explicitly discussed, although such linkages may be used formally or informally in various embodiments. Some examples include: allowing two users to communicate when they are linked to the same subject, two coaches to communicate when they are linked to the same user, allowing coaches to see one or more elements of subject data linked to a user the coach is working with, and the like.

### A. User-Subject Linking

Each user may be linked with no subjects, a single subject, or with multiple subjects. Similarly, each subject may be linked with no users, a single user, or with multiple users. Some examples include:
1. Subject: Individual consumer; User: Individual consumer
2. Subject: None; User: Individual consumer (e.g., a consumer interested in exploring educational materials)
3. Subject: Individual consumer; Users: Individual consumer, primary health care physician, therapist
4. Subjects: Multiple patients in a care practice; User: Primary health care physician
5. Subjects: Individual consumer, consumer's child, consumer's pet; User: Individual consumer
6. Subjects: Multiple animals on a farm; User: Farm owner
7. Subjects: Multiple plants on a farm; User: Farm owner

### B. User-Coach Linking

Each user may be linked with no coaches, a single coach, or with multiple coaches. Similarly, each coach may be linked with no users, a single user or with multiple users. Some examples include:
1. User: Individual consumer; Coach: Diet coach
2. User: Individual consumer; Coach: Diet coach, fitness coach, therapist
3. User: Primary care physician; Coach: Medical specialist
4. User: Farm owner; Coaches: Veterinarian, agricultural specialist

### C. Subject-Subject Linking

One or more subjects may be linked together in various ways. When necessary to distinguish between two subjects who are linked together, the term “pairing” may be utilized and when multiple subjects are linked together, the term “community” may be utilized. The general term “subject-subject linkage” or, if unnecessary to specify, “linkage” may be utilized to refer to either pairings or communities. Samples, subjects, data, or microbiome elements may be linked together in multiple ways to provide a richer and more meaningful analysis. These linkings may be generated in multiple ways and may have multiple representations.

### I. Pairings

One or more subjects may share microbes with each other as a result of many factors. Knowing that a potential microbial sharing exists between different subjects is useful for many reasons. For instance, one reason is because any sampling and analysis method may undersample a subject. Therefore, obtaining a sample from one subject (e.g., using one or more of the described methods) who is linked by microbial sharing to other subjects potentially provides additional information about all linked subjects. For example, if two subjects share a living environment and are linked via microbial sharing, it may be possible to refine estimates of each subject's microbiome (i.e., improve sampling errors). Alternatively, if one subject's sample(s) were obtained at more recent time points, to update and predict changes to the other subject's data. In the language of graph theory, pairings may be considered as edges between subjects (nodes) which may or may not be signed, directed or undirected, weighted or unweighted.

One or more potential microbial sharing linkages could be defined by one or more of the following relationships between two subjects. These relationships include, but are not limited to, one or more of:
1. Shared living environment
2. Shared work environment
3. Shared food or drink
4. Family relationships (e.g., mother and child, etc.)
5. Sexual or romantic partners (e.g., kissing, etc.)
6. Shared pet
7. Shared farm or agricultural setting
8. Shared hospital, lab, veterinary practice or other medical environment
9. Shared group environment (e.g., a church, gym, etc.)
10. Shared food or water supply
11. Shared town, city, country or other municipal or political unit
12. Shared geography
13. Shared environmental factors (e.g., temperature, weather, pollution, etc.)

Another example of a linkage type is a shared subject attribute, allergy, trait, sensitivity, behavior or medical condition. Such shared attribute linkages include, but are not limited to one or more of:
1. A medical condition or shared history such as diabetes, inflammatory bowel disease, heart transplant, etc.
2. A psychological or psychiatric condition such as depression, anxiety, stuttering, bipolar disorder, etc.
3. A particular food allergy, sensitivity, intolerance or preference (e.g., peanuts, legumes, etc.)
4. A particular diet (e.g., vegan, vegetarian, ketogenic, etc.)
5. Age similarity, age range, or other demographic data
6. Gender, gender history, sexual orientation/preference
7. Profession similarity
8. Activity similarity (e.g., same workouts, ambulatory routines, leisure activities, etc.)
9. Religion or political beliefs
10. Race and/or ethnicity
11. Species or breed
12. Individual traits such a handedness, eye color, hair color, fur color, markings, hair texture, earwax type, etc.
13. Multiple subjects associated with one or more of the same users
14. Similar survey data
15. Exposure to similar or same pathogens (e.g., particular diseases) or environmental factors (e.g., pollution)

Another example of a linkage type is one or more shared (or similar) data element(s) between two subjects. These linkages may or may not require the data elements to be shared within a certain temporal distance or data element distance (e.g., using one or more of any data element distances described below). Such shared data element linkages include, but are not limited to one or more of:
1. Shared germline genes, somatic genes, gene expression, proteomic, transcriptomic, metabolomics or other-omics test results
2. Shared medications or treatments between subjects
3. Shared (same or similar) sample data elements, which may include any shared microbiome elements. Examples include, but are not limited to:
   a. The presence or absence of the same or similar microbe, virus, species, genus, phyla or any other taxonomic level or genetic similarity in the microbiome in the two subjects
   b. The presence or absence of the same or similar relative microbiome populations between subjects
   c. The presence or absence of the same or similar metabolites
4. Temporal or data element distance between two subjects
5. Temporal or data element sample location similarities between two subjects
6. Any same or similar context factor or derived quantity a. For example, two similar features (e.g., imaging biomarkers, organ sizes) derived from medical images of the two subjects
7. Any derived analysis quantity of sample data or context factor (e.g., similar alpha diversity, similar subject predictions)
8. The same or similar temporal changes in the subject microbiomes

Pairings may also be generated between two subjects via any number of graph-theoretical generation methods for a set or subset of points (subjects) which are represented by a set or subset of their data elements or a dimensionality-reduced representation (see below). Examples of such pairings may include, but are not limited to:
1. K-nearest-neighbors (using Euclidean distance, minimax distance, mutual information distance, total variation distance, Lo distance, etc.)
2. Fully connected graph
3. Delaunay Triangulation
4. Random pairing assignments between two subjects with a probability of assignment based on one or more methods, including but not limited to any of the weighting techniques described above, uniform probabilities, etc.

One or more weights for each pairing may be derived from one or more methods, including but not limited to:
1. Any one of a distance metric (e.g., Euclidean) defined to describe similarity of shared data elements between subjects. For example, the similarity in diets between one pair of subjects may be different than the similarity in diets between a second pair of subjects and therefore these pairings might be weighted differently.
2. Derived similarity measures from subject data analysis (e.g., alpha diversity, beta diversity, etc.)
3. Input by one or more subjects (users). For example, a pairing between two subjects who are always together in the same household may be weighted differently than a pairing between two subjects who are only sometimes together in the same household (e.g., due to travel, multiple living arrangements, etc.).
4. Amount of time together and/or frequency of interaction between the subjects. For example, frequency of sexual contact.

Any of these linkages may be generated by one or more methods. The methods to generate a linkage may include, but are not limited to, one or more of the following mechanisms:
1. Input by one or more subjects (or users)
2. Location data (e.g., GPS, wearable device locations, IP addresses, etc.)
3. Wearable device measurements
4. Social network links
5. Social media and/or dating application linkages indicating a shared microbial environment (e.g., family, coworkers, romantic relationships, two subjects in the same photo, etc.)
6. Internet usage data (e.g., IP addresses, searches, etc.)
7. Insurance data
8. Governmental data
9. Census or other public data
10. Employer data
11. Survey data
12. Hospital, care provider and/or medical record data
13. Collected subject sample data, context data and/or analysis of this data
14. Inferred data about one or more subjects based on linkages
15. Account data
16. Diet or food journals
17. Farm and agricultural data
18. Family history

Note that one or both subjects (or users concerning the two subjects) may or may not need to agree to a linkage (or removal of a linkage) in order for a linkage to be included (removed) in the subsequent analysis. Linkages may or may not be added, removed or refined over time in response to new data, modified data or user inputs.

For clarity, these subject-subject linkages may or may not be associated with different subtypes to represent different types of subject-subject linkage (e.g., family members, residents of the same town, etc.). This linkage subtype information, if available, is considered part of any linking data or subject data with this sort of linkage.

### II. Communities

As with pairings, communities may represent a group of subjects containing more than two subjects. One or more communities may be defined via many methods, including but not limited to one or more methods described above to define pairings. For example, all members of a household, including pets, may be considered as a community. In this example, one member of the household could also belong to a second, distinct community defined by a workplace environment shared with other subjects. In the language of hypergraph theory, communities may be described as triplets, cycles, hyperedges or sets which may or may not be ordered, directed or signed, weighted or unweighted. Note that in the context of hypergraph theory, the hyperedges representing communities may be of one or more different cardinalities.

### III. Derived Pairings and Communities

In addition to the above methods for generating pairing and community structures, embodiments of the application may also derive pairings from communities and vice versa. Methods for generating pairings from one or more communities include, but are not limited to:
1. Defining a pairing between every two subjects in a community
2. Defining a pairing based on a defined number or percentage of the most similar or dissimilar pairs of subjects in a community
3. Pairwise intersections between two distinct communities

Methods for generating communities from a set or subset of subject pairings include, but are not limited to:
1. The entire set or subset of subjects assigned to one community
2. Graph clustering techniques, in which one or more clusters is defined as a community. Clustering techniques include but are not limited to:
   a. Harmonic clustering
   b. Geometric clustering
   c. Isoperimetric clustering
3. Rotation table methods for identifying a set of cycles from an embedding of the graph
4. Algebraic methods for defining a minimum basis set (e.g., a minimum cycle set defined by Horton's algorithm)
5. A cycle double cover

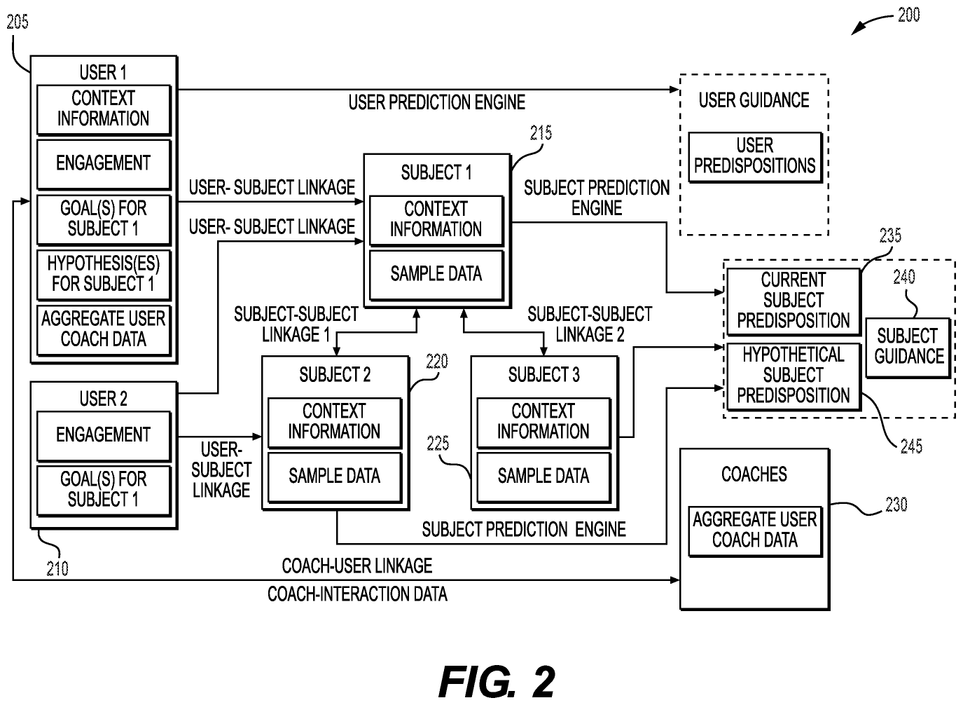

FIG. 2 illustrates a block diagram 200 depicting exemplary associations/linkages that may be present between entities involved in the health optimization system 100. For instance, FIG. 2 provides an indication of: potential linkages between subjects (i.e., commonalties between subjects that bind those subjects in a pairing or community), how users may leverage the subject data associated with one or more subjects to achieve an objective (e.g., to achieve a goal for a subject), how predispositions may be generated for users and/or subjects (e.g., via utilization of user and/or subject prediction engines), and how one or more coaches may be assigned to a particular user based upon their intended needs/goals.

As a non-limiting example of the foregoing, FIG. 2 provides Users 1 and 2 205, 210. User 1 205 may be linked to Subject 1 215 and, in this instance, User 1 205 may be the parent of Subject 1 215. Data associated with Subject 1 215 may include various types of context information and microbiome sample data. User 1 205 may have access to all of the data for Subject 1 205, including: the ability to view and set goals for Subject 1, view a hypothesis for Subject 1 based on their available data and the goals, aggregate user coach data (e.g., communications between the user and the coach 230, etc.), current subject predisposition data 235 data, and subject guidance data 240 that is based on the current subject predisposition data 235. In an embodiment, a user prediction engine may enable User 1 to receive user guidance 245 related to how they may be able to help Subject 1 achieve their goals. User 2 210 may also be linked to Subject 1 215, as well as Subject 2 220, but may have reduced engagement and access permissions with both.

In an embodiment, Subjects 1, 2, and 3 215, 220, 225 may all be linked and may be members of a singular community (e.g., Subjects 1, 2, and 3 215, 220, and 225 may all be members of a group that are frequently around one another). The shared associations between the subjects may enable an unlinked user, such as User 1, to be apprised of hypothetical subject predisposition data 245 of Subjects 2 and 3 220, 225.

### IV. Subject Data

In this section, different types of data are detailed that may or may not be associated with a subject. These context factors, measurements, linkages, and the possible time/location of these data points in relation to a subject are collectively referred to as “subject data” and each specific type of data as a data “element”. For clarity, the subject data for different subjects may or may not contain all the same types of data elements and each subject may or may not contain any or all data elements. For clarity, note that subject data for a certain subject may or may not be considered to encompass any or all user data from one or more users that are linked with that subject and any or all user-subject and subject-subject linkages. For clarity, subject data may or may not include microbiome data. For clarity, note that “element” is used in a similar manner to refer to particular data elements for user data and/or coach data as well.

### A. Subject Context Factors

Some data about a subject are not microbiome related, but rather, may be supplied to the system by a user or via other means. The intention of these context factors is to provide the system with additional information that can enable more accurate and reliable predictions to inform the user about the subject's health and disease.

A variety of potential context factors to be obtained are contemplated herein. Some of these context factors can be obtained either via questionnaires for the user about the subject or via connection of the system with other records such as a smartphone, wearable device, home health device, electronic medical record (EMR), social media accounts, other company, etc. These subject factors may represent data obtained from one or more time points or may represent continuous monitoring data. These context factors about the subject may include, but are not limited to, one or more of the following examples:
1. Age (birthday)
   a. Noticeable changes and/or decline with age
2. Ethnicity or race
3. Breed (e.g., for pets, livestock, other animals, etc.)
4. Strain, specialty seed and/or genetically modified organism (e.g., for plants)
5. Gender history and status
6. Occupation
   a. Title
   b. Role and responsibilities
7. Sexual orientation
8. Sexual history
   a. Kissing
   b. Intercourse
   c. Other sexual activities
9. Relationship status (e.g., single, married, divorced, in a relationship, etc.)
10. Pregnancy status or history
   a. Presence of gestational conditions
   b. Birth control history
11. Shopping, search or online data history
12. Languages spoken
13. Religion or political beliefs
14. Socioeconomic status and history
15. Education status and history
16. Income status and history
17. Payment information (credit card, etc.)
18. Height
19. Weight history and status
20. Circumcision status
21. Contact information (email, phone number, physical address, etc.)
   a. Emergency subject contact information
22. Insurance information
23. Hygiene and/or hygiene frequency, product usage
   a. Handwashing
   b. Flossing
   c. Antibacterial soaps, creams and cleaning materials
   d. Douche usage
   e. Air purifiers
   f. Water chlorination and/or purification
   g. Bathing type and frequency
   h. Living conditions
   i. Tooth brushing
   j. Mouthwash
   k. Bad breath
24. Goals input by one or more linked users
   a. Weight loss or gain
   b. Clinical care and/or mental health support of one or more subjects
   c. Effective agricultural management of one or more subjects
      i. Achieving best agricultural yield from one or more subjects
   d. Fitness goals
      i. Improved endurance
      ii. Strength/muscle mass
      iii. Sexual performance
   e. Improvement to various medical conditions
      i. Pain
      ii. Diarrhea
      iii. Bacterial vaginosis
   f. Change to cosmetic condition
      i. Reduced acne
      ii. Softer skin
      iii. Dry skin
   g. Improvement to a dietary response
      i. Allergy
      ii. Intolerance
      iii. Sensitivity
      iv. Glucose increase
   h. General wellness
      i. Minimizing disease risk
      ii. Improving sleep quality
      iii. Disease resistance
   i. Improved mammary function
      i. Breastfeeding
      ii. Diary animal yield
   j. Microbiome alteration
25. Breastfeeding
   a. Frequency, source for the subject as an infant
   b. Frequency for the subject as a supplier of breastmilk
26. Method of birth
   a. Vaginal
   b. C-section
   c. In vitro
27. Medical history and status
   a. Trauma
      i. Origin
      ii. Treatment
   b. Any or all medical conditions or disorders
      i. Diagnosis
      ii. Diagnosis time and method
      iii. Treatment history
      iv. Treatment response
      v. Current illnesses or conditions
      vi. Pain
         1. Location
         2. Severity
         3. Frequency
   c. Blood test results
      i. Triglycerides
      ii. Creatine Kinase
      iii. Testosterone
      iv. Free Testosterone
      v. Hemoglobin A1c (HbA1c)
      vi. Low density lipoprotein
      vii. High density lipoprotein
      viii. White Blood Cell Count
      ix. Potassium
      x. Alanine Aminotransferase (ALT)
      xi. Red Blood Cell Count
      xii. Hematocrit
      xiii. Mean Cell Volume
      xiv. Mean Cell Hemoglobin
      xv. Mean Cell Hemoglobin Concentration
      xvi. Red Cell Distribution Width
      xvii. Platelets
      xviii. Mean Platelet Volume
      xix. Monocytes
      xx. Neutrophils
      xxi. Lymphocytes
      xxii. Eosinophils
      xxiii. Basophils
      xxiv. Blood glucose level
      xxv. Low-density Lipoprotein (LDL)
      xxvi. High-density Lipoprotein (HDL)
      xxvii. Dehydroepiandrosterone sulfate (DHEAS)
      xxviii. Heavy metals analysis
      xxix. Thyroid function
      xxx. Vitamins and minerals
      xxxi. Melatonin
      xxxii. Cortisol
      xxxiii. Fatty acids
   d. History of blood transfusions
   e. Development disorders
      i. Autism and Asperger
   f. Metabolomic testing results
   g. Presence of chronic conditions and history
      i. Diagnosis and treatment
         1. Date of diagnosis
         2. Method of diagnosis
         3. Treatment
         4. Treatment response
      ii. Inflammatory conditions
         1. Inflammatory bowel disease
         2. Irritable bowel syndrome
         3. Arthritis (rheumatoid, gout, psoriatic)
         4. Dermatitis
      iii. Asthma
      iv. Diabetes (type 1, 2)
   h. Congenital diseases
      i. Cystic fibrosis
      ii. Down's Syndrome
   i. Cardiovascular disease and history
   j. Liver disease and history
   k. Gastrointestinal disease and history
      i. Ulcers
      ii. Lesions
   l. Kidney disease and history
      i. Kidney stones
   m. Sexually transmitted disease history and status
   n. Antibiotic usage and history
      i. Type
      ii. Concentration
      iii. Method of administration
      iv. Date
      v. Reactions
   o. Medications
      i. Frequency
      ii. History
      iii. Response
   p. Skin conditions
   q. Infectious disease status and history (viral, bacterial, prion, etc.)
      i. Type of infection
      ii. Infectious agent
      iii. Treatment
      iv. HIV/AIDS status
      v. Sexually transmitted diseases and/or infections
      vi. Whether one or more infections occurred shortly prior to a sustained change in the subject
   r. Neurological disease and disorder
      i. Alzheimer's disease
      ii. Mild cognitive impairment
      iii. Parkinson
      iv. Multiple sclerosis
   s. Previous surgeries
      i. Removal
         1. Tonsils
         2. Adenoids
         3. Appendix
         4. Tumor
      ii. Organ transplant
         1. Transplant donor context information and/or measurement data
         2. Transplant response and success
      iii. Type
      iv. Date
      v. Response
   t. Bariatric surgeries or treatments
      i. Type
      ii. Date
      iii. Response
   u. Cancer history
      i. Presence of cancer
      ii. Type of cancer
      iii. Diagnostic method
      iv. Treatment
      v. Recurrence
   v. Genomic test results for one or more elements of tissue extracted
   w. Genetic test results
      i. Germline
      ii. Somatic
   x. Proteomic test results
   y. Transcriptomic test results
   z. Diagnostic testing results
      i. Glucose
      ii. Cortisol
      iii. Sex-Hormone Binding Globulin
      iv. Albumin
      v. Calcium
      vi. Magnesium
      vii. Hemoglobin
      viii. Aspartate Aminotransferase (AST)
      ix. Gamma-glutamyl Transpeptidase (GGT)
      x. Sodium
      xi. High Sensitivity C-Reactive Protein
      xii. Ferritin
      xiii. Total Iron Binding Capacity
      xiv. Iron
      xv. Transferrin Saturation
      xvi. RBC Magnesium
      xvii. Folate
      xviii. Vitamin B12
      xix. Vitamin D
   aa. Radiology
      i. Radiology reports
      ii. Features derived from radiology images
   bb. Pathology
      i. Pathology reports
      ii. Features derived from pathology images
   cc. Vital measurements
      i. Resting heart rate
   dd. Dental
      i. Dental procedures
      ii. History
      iii. Fillings
28. Mental history
   a. Diagnosis of psychological or psychiatric disorder
      i. Brain fog
      ii. Biopolar
      iii. Fatigue
         1. Chronic
         2. Episodic
      iv. Stress
      v. Depression
      vi. Anxiety
      vii. Psychosis
   b. Therapy duration, history and type
   c. Response to therapy
29. Time and date
30. Diet history and status
   a. Water amount and frequency
   b. Nutritional content of food and drink
      i. Presence and/or amount of specific ingredients and/or nutritional elements of food and drink
   c. Frequency of meals
   d. Specific diet followed at present or in the past/future
   e. Fasting
   f. Caffeine amount and/or frequency
   g. Alcohol amount and/or frequency
   h. Drug amount and/or frequency
   i. Chewing tobacco amount and/or frequency
   j. Appetite and/or appetite changes
   k. Time of day when food is ingested
31. Menstrual history and status
32. Heartbeat
33. Living situation and condition
   a. House
   b. Apartment building or other communal living
   c. Wilderness
   d. Urban homelessness
   e. Farm
   f. Nursery
   g. Dwelling level (e.g., 1st floor, 2nd floor, etc.)
   h. Access to sunlight
   i. Number and locations of places for defecation
   j. Number, characteristics and/or frequencies of people, animals and/or plants lived with
   k. Age and size of dwelling
   l. Level of subject confinement within living situation
   m. Air quality within living situation
      i. Air composition (amounts or proportions or different gases or volatile compounds) before, during, and/or after defecation
         1. Methane
         2. Radon
         3. Smoke
      ii. Presence of mold or other airborne microbes
34. Allergies
   a. Food
      i. Intolerance
      ii. Sensitivity
   b. Environmental
      i. Intolerance
      ii. Sensitivity
35. Geographic location
   a. Latitude
   b. Longitude
   c. Altitude
   d. Zip code
   e. Town
   f. City
   g. Country
   h. State
   i. Province
   j. Farm
   k. Institute
   l. Environmental disaster history
      i. Flood
      ii. Earthquake
      iii. Fire
      iv. Tornado
      v. Volcanic eruption
   m. Epidemiological information associated with location
      i. Local environmental factors
      ii. Disease or disorder prevalence
      iii. Local population data
36. Weather history and status
   a. Temperature
   b. Humidity
   c. Barometric pressure
   d. Air quality
   e. Pollution or smog
37. Presence of and type of pet, including frequency of contact and/or living arrangement
38. Exposure to livestock
39. Heredity
40. Smoking (and/or vaping) history and current frequency/habits
41. Alcohol history and current frequency/habits
42. Drug usage history and current frequency/habits
43. Sleep patterns and current frequency/habits
   a. Time of day when subject falls asleep and/or wakes
   b. Sleep interruptions and time of sleep interruptions
44. Family medical history
   a. All medical history factors above for one or more relations
   b. Relationship between the subject and one or more family members
45. Sun exposure and use of sunscreen history
   a. Frequency and intensity of sun exposure
   b. Use of sunscreen frequency, location, brand and/or type
46. Vitamins and supplements being taken
   a. Vitamin or supplement type, concentration and/or brand
   b. Probiotics, type, concentration and/or brand
   c. Prebiotics, type, concentration and/or brand
   d. Postbiotics, type, concentration and/or brand
47. Cosmetics or beauty product type, concentration, location of application, provenance, history of usage and/or brand
48. Subject behaviors
   a. Ability to complete tasks
   b. Procrastination
49. Fitness and exercise
   a. Type
   b. Frequency
   c. Exercise patterns
   d. Body measurements
      i. Waist circumference
      ii. Hip circumference
      iii. Body surface scanning
      iv. Body fat percentage
      v. Muscle percentage
      vi. Body mass index
      vii. Water retention
      viii. Bio-impendence measurements
50. Gasterointestinal wellness
   a. Urination
      i. Frequency
      ii. Pain associated
      iii. Color
   b. Defecation
      i. Frequency
      ii. Pain associated
      iii. Color
      iv. Stool type
      v. Time of defecation
      vi. Duration of defecation
   c. Burping frequency and/or severity
   d. Hiccup frequency and/or severity
   e. Bloated feeling frequency and/or severity
   f. Gas frequency and/or severity
   g. Heartburn frequency and/or severity
51. Local soil, geology, water, air, environmental or atmospheric samples
   a. Processed in a lab or referenced in an existing database to inform
      i. Quality
      ii. Composition
      iii. Microbial concentrations and related properties
52. Mortality
   a. Subject death
   b. Cause of death
   c. Time of death
53. Livestock production, yield, effectiveness, quality, outputs, characteristics, etc.
   a. Milk
   b. Meat
   c. Fur
   d. Leather
   e. Wool
   f. Honey
   g. Work capacity
   h. Organ quality (e.g., for transplantation)
   i. Blood and/or other biofluids
   j. Mating or breeding
   k. Pest resistance
   l. Growth hormones
54. Crop production, yield, effectiveness, quality, outputs, characteristics, etc.
   a. Crop density
   b. Pest resistance
   c. Crop yield and quality
   d. Fertilizers used
   e. Crop rotation
   f. Equipment used
55. Subject DNA, RNA, transcriptomics, proteomics, cell free DNA etc.
   a. Sequencing data
   b. Genetic markers
   c. Genomic analysis of one or more tumors or abnormalities
56. Behavioral habits
   a. Smartphone usage patterns
      i. Screen on/off patterns
      ii. Screen locked/not locked patterns
      iii. Battery level patterns
      iv. GPS strength patterns
      v. Number of Bluetooth devices connected patterns
      vi. Wifi compared to satellite connection patterns
      vii. Apps installed and usage patterns
   b. Voice data, changes and patterns

For clarity, any of the examples of subject predispositions may also be input as subject context factors. For clarity, any mention of a probiotic, prebiotic or postbiotic may also refer to a combination of these, which is sometimes called a synbiotic.

Note that each of these contextual information elements may also be represented as probabilities (e.g., stochastic variables). One reason to use probabilistic representations is to account for the possibility of inaccurately entered information or information that is not current. For example, a user recording 8 hours of sleep for the subject the night before might be inaccurate or imprecise (perhaps it was 7.6 hours), and therefore a probabilistic representation (e.g., a distribution of possibly correct values informed by the entered information or a probability associated with the entered information) might be more appropriate.

Some of these subject context factors may be in the form of unstructured data, free text, audio recordings, video, etc. For clarity, natural language processing (NLP), image/video analysis and/or sentiment analysis may be applied to one or more of these subject context data to add additional subject context data. For one example, NLP could be applied to a doctor's report in the subject's medical record to assess a subject medical condition. As another example, sentiment analysis could be applied to a doctor's report to assess the doctor's level of concern with a written condition. Any outputs of NLP or sentiment analysis used on subject context data is considered as an additional form of subject context data.

The collection of these context factors may be obtained by one or more methods and stored on an electronic storage device. An exemplary list of methods is provided below, which is not intended to be exhaustive:
1. Survey question answers from one or more users about the subject
2. Inferred context factors from other subjects linked to the subject
3. Account information associated with the subject
   a. Smart phone application account
   b. Website account
   c. Social media account
   d. Paper records of account
4. Electronic Health Records (EHR), Electronic Medical Records (EMR), Personal Health Records (PHR), Consolidated Clinical Document Architecture (CCDA), Picture Archiving and Communication System (PACS), Vendor Neutral Archive (VNA), Health Information System (HIS), Laboratory Information System (LIS), Radiology Information System (RIS) via one or more of any number of standards, including:
   a. Native Application Programming Interfaces (APIs)
   b. Health Language 7 (HL7)
   c. Fast Healthcare Interoperability Resources (FIHR)
   d. A Health Information Exchange (HIE)
   e. Digital Imaging and Communications in Medicine (DICOM)
   f. Tokenized connection to a Real World Data (RWD) or Real World Evidence (RWE) database
5. Directly from medical devices such as a capsule endoscopy
6. Location data associated with a subject, obtained via a smartphone, wearable devices, Internet Protocol (IP) address, Global Positioning System (GPS), etc.
7. Shopping, search or internet usage history obtained via web browser cookies account linking with shopping stores, websites and/or services
8. Census data
9. Public databases, providing a variety of data for a particular location, such as:
   a. Altitude
   b. Current and history of weather, atmospheric, pressure, temperature, air quality, humidity, smog, pollution
   c. Soil, geology, water and air quality, composition and microbial environment
   d. Local environmental disasters, type, magnitude, history e. Epidemiological information
10. Wearable, implantable, carried, attached or home health devices with linked accounts and/or linked to a subject's smartphone (e.g., via Bluetooth) and/or other item that is worn or carried by the subject (e.g., a radio beacon, air tag, etc.), such as:
   a. Smartphones and all smartphone sensors
   b. Tracking beacon (e.g., air tag)
   c. Smart watches
   d. Smart rings
   e. Smart jewelry
   f. Connected pregnancy tests
   g. Smart clothing
   h. Pacemaker
   i. Continuous glucose monitoring
   j. Smart scale
   k. Blood pressure monitors
   l. Sleep monitors (e.g., sleep tracking mat)
   m. Smart thermometer
   n. Accelerometer
   o. Body temperature
   p. Sweat monitor
   q. Heart rate monitor
   r. Pulse oximeters
   s. At home infectious disease testing
   t. Pregnancy tests
   u. Air quality monitor
   v. Bathroom and/or defecation monitoring device
      i. Smart toilet
      ii. Location device to determine when a subject is in the bathroom
11. Additional testing performed separately and linked as part of the inventive concepts or linked via a commercial hospital, lab or provider
12. Insurance or payer information
13. Clinical trial databases
14. From the account of a linked subject to the current subject with a known relationship (friend, family, pet, co-worker, sexual/romantic partner, etc.)
15. Menstrual history (period tracking) and diet tracking applications
16. Farm and/or agricultural records (database)

One or more of these collection devices may or may not be calibrated and the post-calibration data may be used to supply the subject context data. For example, a continuous glucose monitoring device often needs to calibrate to an individual before it can produce reliable readings.

### B. Microbiome Data

This section details the type of microbiome samples, measurements, data and location information associated with the subject at one or more time points. Recall that the term microbiome is utilized herein to refer to the aggregate of all microbiota (including all related microbiota biological properties such as genetics, proteins, metabolites, transcriptomics, etc.) and properties of the environment that they reside on or within human tissues, solids, biofluids and/or biofilms along with the corresponding anatomical sites in which they reside, including the skin, mammary glands, seminal fluid, uterus, placenta, ovarian follicles, lung, saliva, oral mucosa, nasal mucosa, conjunctiva, biliary tract and gastrointestinal (GI) tract. Types of microbiota in our definition include bacteria, archaea, fungi, protists, viruses, phages, plasmids, prions, parasites, mobile genetic elements and micro-animals.

Therefore, the elements of microbiome data being produced or received will depend on the sample taken (e.g., sample size, sample quality, etc.) and the analysis method. Some examples of microbiome data that may be produced or received include, but are not limited to:
1. Microbe species (and other taxonomic level) present
2. Microbe species (and other taxonomic level) quantities
   a. Evidence of bacterial blooming
3. For one or more types of microbiota, the microbe genes, transcriptome, proteome, variants, metabalome, metabolome, mRNA and/or other microbiota-omics, including subsequences and characteristics
   a. Biosynthetic Gene Clusters (BGC)
   b. Evidence of horizontal gene transfer
4. Subject DNA, RNA, cell free DNA etc.
   a. Sequencing data
   b. Genetic markers
   c. Amount and/or proportion of subject DNA, etc. in a sample
5. For one or more types of microbiota, the growth rate of the microbiome element. The growth rate for a microbe species (or other taxonomic element) may be determined by the copy number of DNA in replication
6. Microbial environmental factors such as:
   a. Chemical presence and quantity
      i. Metabolites
      ii. Cytokines
      iii. Amino acids and associated structures
         1. Peptides
         a. Antimicrobial peptides
         b. Known and unknown post-translationally modified peptides
         c. Ribosomally synthesized and post-translationally modified peptides (RiPPs)
         d. Non-Ribosomal Peptides (NRPs), (e.g., lugdunin, surugamides)
         2. Proteins
         a. Zonulin
      iv. Nutrients
         1. Dietary minerals
         2. Human milk oligosaccharides
         3. Glycerol
         4. Polyphenols
         5. Macro nutrients (protein, fat, carbohydrates)
         6. Micronutrients
         7. Short-chain fatty acids
         8. Alcohol
      v. Neurotransmitters
      vi. Hormones
      vii. Acids
         1. Salivary uric acid
      viii. Foreign bodies in solution (e.g., microplastics)
      ix. Gases
      x. Antibodies
      xi. Medication
         1. Antibiotics
         2. Illegal drugs
         3. Stimulants
      xii. Semiochemicals
      xiii. Electrolytes
   b. Temperature
   c. Pressure
   d. Alkalinity and/or pH
   e. Inflammation
   f. Properties or characteristics of the surrounding tissue
      i. Ulcers
      ii. Lesions
      iii. Mucosal layer condition
      iv. Intestinal permeability
   g. Bleeding
   h. Enzymes
   i. Bile
   j. Ionic concentrations
   k. Imaging of the local tissue
      i. Optical
      ii. Spectroscopy
      iii. Ultrasound

For clarity, the term “species” is utilized to refer to both known (identified) species and/or unknown or genetically distinct microbiota (and/or phylotypes). Genetically distinct may be defined by either an exact genetic match or a genetic similarity measure.

Each element of microbiome data may also include one or more sample data for the one or more samples associated with the element of microbiome data. This sample data may include one or more of a range of different information about the sample, including but not limited to the sample:
1. Time
2. Location, which may or may not be at one or more different levels of precision (e.g., “GI tract”, “2.37 mm distal to the pyloric sphincter” and/or “coordinate (2.2, −7.9) on a subject skin surface model”)
3. Type
4. Device
5. Analysis method
6. Operator
7. Data origin (e.g., if the sample data was received from an external source)

### I. Sample Types

Given the optional one or more context factors for a subject, the next step may be to acquire or receive one or more bodily samples of the subject from one or more locations at one or more times and to associate each sample with one or more times and locations. These samples may include, but are not limited to:
1. Fluids, solids or biofilms from the GI tract
   a. Saliva
   b. Plaque
   c. Chyme
   d. Bile
   e. Stomach acids
   f. Digestive juices
   g. Pancreatic fluids
   h. Feces/stool
   i. Mucus
   j. Blood
   k. Tissue samples (biopsies)
2. Skin tissue samples
3. Skin swab samples
4. Tissue biopsies or biopsies
5. Oral, GI-tract mucosa swab samples or biopsies
6. Vaginal or uterine fluid, swab samples or biopsies
7. Urine
8. Penile samples
9. Nasal swabs or biopsies
10. Breast milk or biopsies
11. Dental samples
12. Lung outputs or biopsies
13. Conjunctiva samples
14. Biliary tract samples

### II. Sampling Mechanisms and Devices

In order to obtain one or more samples for the patient, one or more of several different types of sampling device that may be used. In some cases, the samples are obtained manually and may be analyzed either in a central lab and/or a point-of-care (PoC) ex vivo diagnostic device. In some cases, the samples must be retrieved after sampling (e.g., a GI-ingestible pill that performs sampling and requires retrieval from stool after passing) for analysis ex vivo. In some cases, the samples may be analyzed on the device (e.g., a smart endoscope, a GI-ingestible capsule, etc.) in vivo and transmitted to a receiver (e.g., via an antenna, Bluetooth, NFC, etc.) for processing and storage and/or retrieved ex vivo for data retrieval. Both ex vivo and in vivo analysis of the one or more subject samples may be performed and there may be different sample collection and/or sample analysis methods for different samples, where each sample is associated with a time and location.

A wide variety of sampling devices may be used to obtain samples for analysis. Such devices include:
1. Wet (e.g., saline) or dry swabs (e.g., flocked swabs, calcium alginate, rayon, isohelix, etc.) for sample collection (including possible pre-application of agents such as proparacaine) on skin, oral cavity, nasal cavity, conjunctiva, etc.
2. Cytobrush
3. Dipstick
4. Stool samples obtained externally following excretion or internally via one or more of any of various invasive means (e.g., colonoscope).
5. Fluids (e.g., blood, saliva, urine, mucus, phlegm, bile, GI tract fluids, etc.) may be obtained through one or more means. Note that the components and biomarkers in these fluids may be analyzed via a variety of both classical in vitro diagnostics (e.g., cholesterol, blood glucose, troponin, C-reactive protein, etc.) or more recent liquid biopsy techniques (e.g., cell-free DNA, etc.). Fluid sampling may be done through a variety of mechanisms, including but not limited to:
   a. General fluid capture methods (e.g., with categorial location tags such as “circulatory”, etc.), such as would be obtained by venipuncture, finger stick, external collection via excretion, etc.
   b. Invasively obtained (e.g., via a scope)
6. Tissue obtained via one or more means, including but not limited to
   a. Biopsy
   b. Resection
   c. Surgery
   d. Ingestible capsule
   e. Bodily excretion (e.g., in stool, skin flaking, etc.)
   f. Autopsy
7. Gases obtained and analyzed via one or more techniques, including but not limited to:
   a. Analysis from sampled fluids, breath, flatulence, etc. As with context information, gases may be sampled with various means (coated stainless steel canister, end tidal air collector, Tedlar bag, etc.) and analyzed for a variety of volatile organic compounds or inorganic compounds via many different means
   b. Ingestible capsule that performs gas measurement and is later retrieved for ex vivo analysis (or data transfer) following excretion or invasive means (e.g., surgery) and/or performs analysis in vivo and transmits that information to a receiver. In vivo analysis of gases may be performed with one or more of the following methods, including but not limited to:
      i. Ingestible capsule that collects and/or directly analyzes (either of transient material or collected material) fluid, microbiota, biofilms, tissue, gas, temperature, pH, etc. and is either later retrieved following excretion, tether and/or invasive means (e.g., surgery) and/or performs analysis in vivo and transmits that information to a receiver. There are multiple different methods for an ingestible capsule to collect and/or analyze samples, including but not limited to:
         1. Direct ingestion of fluid through an aperture or porous membrane. Such ingestion could be performed via multiple methods that are either entirely passive (due to capsule motion) or via an induced negative pressure, such as that created by osmosis or via an active device (e.g., a small motor), which may or may not include a discharge hole for excess fluid.
         2. A self-polymerizing reaction mixture that entraps microbes and biomarkers
         3. Salt chamber capture (e.g., calcium chloride salt powder)
         4. Sponge
         5. Gas-permeable membrane (e.g., polydimethylsiloxane)
         6. Temperature sensor
         7. Pressure sensor
         8. pH sensor
8. Biofilms (e.g., plaque, mucosal lining, etc.)
   a. Scraping
   b. Excretion
   c. Invasive capture
   d. Ingestible capsule that collects biofilms
9. Imaging
   a. Capsule endoscopy
   b. Spectroscopy
      i. Raman spectroscopy
   c. Colonoscope
   d. Endoscope
   e. Digital (or digitized) optical camera
      i. Color imaging
   f. Digital (or digitized) microscopy

Note that the times and locations of each of these samples may be determined using one or more different methods and technologies, including those on the list above. Each sample may or may not utilize one or more different or similar methods for obtaining time and location information about the sample. Note that in some cases the times and locations for one or more samples may be obtained differently and have different levels of precision. For example, a precisely located and manually recorded skin swab and a categorically located stool excretion (e.g., labeled simply as “colon” or “GI tract”) could both be obtained and analyzed. A hierarchy or taxonomy (ontology) of different locations may or may not be used to define a relationship between different locations and times.

### III. Sample Time and Location

For each sample acquired from the subject, the location and time of the sample may or may not be recorded. These sample times and location may be recorded in one or more multiple different ways. Any of these location methods may be associated with one or more samples and the location may be retrieved wirelessly (e.g., via an antenna, Bluetooth transmitter, etc.) or recorded and accessed when the sample is retrieved (e.g., with a GI-ingestible capsule). For clarity, each individual sample may or may not have multiple time and/or location measurements performed with multiple different time and/or location measurement methods.

Time measurement methods include, but are not limited to:
1. Manual recording of the time of the sample by the individual taking the sample. This manual recording may be done initially in many forms, including on paper, smartphone, audio recording device (voice), electronic form, etc. Eventually, all sample times are recorded and stored electronically on an electronic storage device.
2. A sensor or transmitter that records time of the same when the sample is taken. For example, the sensor may include a clock, connection (wired or wireless) to a time server or many other means to record the time when the sample has been taken. The clock could be calibrated to a universal time or initialized at a particular time and recorded relative to that initialized time (such as with a stop watch).

Location measurement methods include, but are not limited to:
1. Manual recording of the location of the sample by the individual taking the sample. This manual recording may be done initially in many forms, including on paper, smartphone, audio recording device (voice), electronic form, etc. Eventually, all sample locations are recorded and stored electronically on an electronic storage device.
2. A sensor or transmitter that records location in absolute 3D space (world coordinates). For example, such a sensor may assess its position via triangulation with multiple known and calibrated beacons, transponders, etc. A sensor location may also be determined by external in vivo imaging via x-ray, ultrasound, computed tomography (CT), magnetic resonance imaging (MRI), etc. or internally via laparoscopic imaging, optical coherence tomography, etc. In these cases, an opaque device may be attached to the sample collection mechanism to enhance the visibility of the imaging. The location determination via such imaging may be determined in many ways, such as by calibration of the imaging device (e.g., known instrument or patient positioning), manual reading of the images and recording the location (similar to the manual recording method above), automated analysis of a digitized image with algorithms executed by an electronic processor, etc.
   a. A sensor or transmitter that generates location relative to subject coordinates. Relative position coordinates could be multidimensional.
   b. For an example of a three-dimensional coordinate, such a sensor or transmitter could assess position in space relative to one or more known beacons, transponders, to a calibration point placed on or inside the subject, etc. A reference point could also be obtained for the subject via one or more in the vivo imaging methods referred to above and using the imaging to map the location relative to that reference point.
   c. For an example of a two-dimensional coordinate, the sensor or transmitter location could be mapped to the surface of the subject's body (skin) or an internal surface such as the subject's lungs, vagina, uterus, oral/nasal cavity, teeth, conjunctiva, mucosal lining, GI tract, etc.
      i. This surface could have been previously mapped into a digital representation on an electronic storage device through multiple means such as time-of-flight imaging, structured light imaging, in vivo imaging such as x-ray, ultrasound, CT, MRI, sensor network (e.g., smart clothing), multiple point probes together with surface reconstruction algorithms. The location of the sample could be identified relative to this surface via either the same means used to create the surface (e.g., in vivo imaging of the sample on the surface) or mapped to the closest point on the surface if both the surface and the sample are located in the same coordinate system (world coordinates or relative coordinates).
   d. For an example of a one-dimensional relative coordinate, a biological structure could be approximated by a one-dimensional space. For example, the GI tract could be approximated as a one-dimensional curved line (e.g., using the centroid of the cross-section of the GI tract to define a curved line) and determining the sensor or transmitter position along this one-dimensional space as a geodesic distance from a pre-defined origin location (e.g., mouth, anus, etc.). The sensor or transmitted position in the space could be determined via various means, such as
      i. Using ex vivo imaging to measure the location of the sensor or transmitter and projecting that location onto the one-dimensional space.
      ii. Measuring time elapsed since introduction of the sensor/transmitter and using accelerometer or velocimeter measurements (or assuming a predefined or user-input defined constant velocity) to determine travel within the approximately one-dimensional space.
3. A sensor or transmitter that generates a categorical location relative to the subject. In some cases, manual recording may be used to assess the categorical location of the sample. In other cases, the anatomical location may also be determined automatically by measuring biological characteristics surrounding the sample acquisition, such as pH, local gas composition (oxygen, oxygen-equivalent concentration profile, hydrogen, nitrogen, carbon dioxide, etc.), sweat, temperature, etc., and matching those measured biological characteristics with known biological characteristics of different locations. For example, a sample acquired in the GI tract that measured a surrounding pH between 1.5-3.5 can be determined to have been taken in the stomach. Categorical locations may be defined narrowly (e.g., proximal duodenum) or broadly (e.g., GI tract) and may be defined in an anatomical or functional taxonomy describing relationships between different categorical locations (e.g., proximal duodenum is part of duodenum which is part of the small intestine which is part of the GI tract, etc.). Examples of categorical locations include but are not limited to:
   a. Any anatomical locations
   b. Particular teeth (or locations on the teeth) where the sample was obtained
   c. Particular fingers, hands, nose, forehead, etc. where a skin sample was obtained
   d. Particular sections or locations in the GI tract such as mouth, esophagus, stomach, pyloric valve, appendix, biliary ducts, duodenum, jejunum, Ileum, cecum, ascending colon, right colic flexure, transverse colon, left colic flexure, descending colon, sigmoid colon, rectum, anal canal, etc.
   e. Particular locations in the oral cavity, such as lips, tongue, salivary ducts, nasal cavity, pharynx, epiglottis, larynx, etc.
4. A location-activated switch that triggers sample collection at a particular location. A location-activated switch using any of the same mechanisms (such as those below) could also be used to turn off sample collection and therefore limit sample collection to a target location or set of locations. By specifying a location-activated switch, the system knows that the sample was taken from the location specified by the switch. In this case, the term switch refers to any change of device state that initiates sample collection. There are many such types of location-activated sample collection switches, which may include but are not limited to the following mechanisms:
   a. Manual triggering of sample collection through the use of visual inspection, in vivo imaging or tethering of the sample collection device (e.g. location tethered biopsy, tethered endoscopy, etc.).
   b. The sample collection of a GI-ingestible device could be activated after a certain time (e.g., by electronically or magnetically opening/closing gates to allow fluidic capture) and the location could be determined by assessing the expected location of the device after a certain time (possibly also using accelerometer or velocimeter readings). Similarly, a sampling device passing through the vagina or across the skin with known or estimated speed and initiated at a particular time and location could be used to locate a sample taken after a certain time has elapsed via the corresponding time stamp.
   c. A GI-ingestible device could be coated such that the coating dissolves at a target location (due to pH, enteric coating, etc.). One such non-limiting example is Cellulose Acetate Phthalate.
   d. The switch of a sampling device could be made to trigger electronically by any biological profile associated with a certain location, such as pH, local gas composition (oxygen, hydrogen, nitrogen, carbon dioxide, etc.), local chemical concentration, local microbiome sampling composition, local sweat, local temperature, etc. A switch could also be made to trigger mechanically via different mechanisms in response to a biological profile, such as a hydrogel that responds by swelling to initiate or terminate sample collection.
   e. Magnetically opened and closed sampling (triggered) for sampling at different locations
   f. Electrically powered sample collection that is triggered by a magnetic reed switch or exposure to gastric acids to generate power
   g. A machine learning or statistical method could be trained by collecting a series of known locations and biological profiles to identify a location by a biological profile and to trigger the sample collection when the biological profile was determined to match the biological profile of a known location.
   h. Active movement of the sampling device to a target location via a control mechanism. This control mechanism could be performed via a range of methods, including manual manipulation, actuators controlled via wired or wireless controllers, moving the sampling device with one or more external magnets, etc. This control mechanism may or may not include feedback for the sampling operator.

In each of these cases, the sensor or transmitter could be attached to the sample or sample acquisition device. As with all devices described herein, it is presumed that these sensors, transmitters, receivers, transponders, tethers, magnets, controllers, actuators, etc. are appropriately calibrated and sterilized, if necessary.

### IV. Sample Analysis

Depending on the sample type, sample device, and aspect of the microbiome being analyzed the sample may be analyzed with one or more of a large number of in vivo and/or ex vivo methods. In the following, examples are detailed regarding how the microbiome samples might be analyzed for each of the microbiota and/or microbiota environment. For clarity, sample analysis may be performed as part of the invention and/or received from an external source.

### i. Sample Analysis of Microbiota

A sample analyzed in vivo (e.g., ingestible capsule) or ex vivo (e.g., a lab and/or point-of-care device) can use a wide variety of different methods to analyze the microbiota, both multi-omic and classical. Examples of these in vivo and/or ex vivo methods potentially include, but are not limited to, one or more of the following methodologies:
1. Culturing (e.g., streak plate culturing)
2. Visual and/or automated inspection with a microscope (conventional and/or digital scanning device) and measurement
3. Temperature, alkalinity (pH) and gas sensors
4. High throughput isolation (culturomics)
5. Molecular methods
   a. Targeted or untargeted panels
   b. Enzyme-linked immunosorbent assays (ELISA)
   c. Metabarcoding
      i. Barcoding
         1. Mitochondrial gene cytochrome oxidase 1 (CO1)
         2. Ribosomal DNA (rDNA)
         a. 16S
         b. 18S
         c. 12S
         d. Cytochrome B
      ii. DNA extraction
         1. DNA extractions and purifications
         2. Amplicon generation
         3. Primer mixtures
         4. Labeled nucleotides
         5. Polymerase chain reaction (PCR) amplification
      iii. Sequencing
         1. High throughput
         2. Sangar sequencing
         3. Next Generation Sequencing (NGS)
         4. Third Generation sequencing
         5. Nanopore sequencing
      iv. Data analysis
         1. Bioinformatic matching of outputs to databases
         2. Pruning
   d. Metagenomic sequencing
      i. Shotgun sequencing
      ii. 454 pyrosequencing
      iii. Chromosome conformation capture
      iv. Sequence pre-filtering
      v. Assembly
      vi. Gene prediction from a database (e.g., basic local alignment search tool (BLAST) searches)
      vii. Binning
      viii. Data integration
   e. Metatranscriptomic sequencing
      i. RNA extraction
      ii. Messenger RNA (mRNA) enrichment
         1. Removing ribosomal RNA (rRNA) through ribosomal RNA capture
         2. Using a 5-3 exonuclease to degrade processed RNAs (mostly rRNA and transfer RNA (tRNA))
         3. Adding polyadenylation (poly(A)) to mRNAs by using a poly(A) polymerase (e.g., in E. coli)
         4. Using antibodies to capture mRNAs that bind to specific proteins
      iii. Complementary DNA (cDNA) synthesis
      iv. Preparation of metatranscriptomic libraries
      v. Data analysis
         1. Mapping reads to a reference genome database
         2. Perform de novo assembly of the reads into transcript contigs and supercontigs
      vi. Microarrays (e.g., tiling microarrays) or RNA-Seq may also used to measure microbial transcription levels, to detect new transcripts and to obtain information about the structure of mRNAs (e.g., the untranslated region (UTR) boundaries)
   f. Metaproteomics
      i. Shotgun proteomics
      ii. Two-dimensional polyacrylamide gel electrophoresis
      iii. Mass spectroscopy (MS) peptide identification
      iv. Gel-based (one-dimensional and two-dimensional) and non-gel liquid chromatography based separation
      v. Gene expression measurement
      vi. Assessment of protein structural information
   g. Metabolomics, metabonomics, exometabolomics
      i. Shotgun lipidomics
      ii. Analyte separation
         1. High performance liquid chromatography (HPLC)
         2. Gas chromatography (GC)
         3. Two-dimensional gas chromatography
         4. Electrospray ionization
         5. Capillary electrophoresis
         6. Flame ionization detection
      iii. Metabolites extraction with the addition of internal standards and derivatization
      iv. Metabolite detection and quantification
         1. Liquid chromatography
         2. Gas chromatography (GC)
         3. Mass spectroscopy
         4. Nuclear magnetic resonance (NMR) spectroscopy
         5. Electron ionization
         6. Atmospheric-pressure chemical ionization
         7. Electrospray ionization
         8. Secondary electrospray ionization
         9. Surface-based mass analysis
         10. Nanostructure-Intiator MS
         11. Matrix-assisted laser desorption/ionization (MALDI)
         12. Secondary ion mass spectrometry
         13. Desorption electrospray ionization
         14. Laser ablation electrospray ionization
         15. Ion trap (e.g., orbitrap) MS
         16. Fourier-transform ion cyclotron resonance
         17. Ion-mobility spectrometry
         18. Electrochemical detection (e.g., coupled to HPLC)
         19. Raman spectroscopy and radiolabel (e.g., combined with thin-layer chromatography)
      v. Metabolite feature extraction and data analysis
         1. Identification of a metabolite according to fragmentation pattern
         2. Digitized spectra
         3. Metabolite features
         4. Toxicity assessment
         5. Functional genomic assessments
         6. Fluxomic assessment
         7. Nutrigenomic assessment
6. 16S, 18S or 12S rRNA sequencing and analysis
7. Force measurement sequencing
8. FPGA acceleration (basecalling) for nanopore sequencing
9. Biosensors and/or coupled with readout sensors (e.g., miniaturized luminescence, etc.)
   a. One or more bacteria, probiotics or other biosensors may be designed and created via synthetic biology techniques to target response to one or more elements of the microbiome (e.g., microbes, metabolites, viruses, phages, etc.).
   b. Additional mechanisms include other biosensors such a protein biosensors that can be constructed from a system with two nearly isoenergetic states, the equilibrium between which is modulated by the analyte being sensed (e.g., LucCage and LucKey). Such biosensors may be designed to target response to one or more elements of the microbiome. These responses may be sensed, then recorded and/or transmitted to identify the targeted microbiome element.
   c. One possible readout mechanism is for the bacteria or other biosensors to luminesce in response to the targeted microbiome element, which is detected by a photodetector which may or may not transmit the detection event to an outside receiver. In such a case, the biosensor probiotics could lie adjacent to readout electronics in individual wells separated from the outside environment by a semipermeable membrane that confines cells in the device and allows for diffusion of small molecules or other targeted elements of the microbiome.
   d. Enzyme catalyzation to create color or electrical output that may be coupled with readout sensors

Note that subject samples may also be sampled and analyzed in vivo, where the data is either retrieved ex vivo or where the data is transmitted to an external receiver (e.g., via an antenna, Bluetooth, etc.) for processing and storage from the sampling device. Each sample may be independently analyzed either in vivo, ex vivo, received from an external source or a combination of these.

### ii. Sample Analysis of Microbiota Environment

A sample analyzed in vivo (e.g., ingestible capsule) or ex vivo (e.g., a lab and/or point-of-care device) can use a wide variety of different methods to analyze the microbiota, both multi-omic and classical. Examples of these in vivo and/or ex vivo methods potentially include, but are not limited to, one or more of the following methodologies:
1. Visual and/or automated inspection with a microscope (conventional and/or digital scanning device) and measurement
2. Collected gas information (e.g., hydrogen, methane, oxygen, etc.) may be analyzed via one or more techniques, including but not limited to:
   a. Analyzed for a variety of volatile organic compounds or inorganic compounds via many different means, such as:
      i. Proton Transfer Reaction Mass Spectrometry
      ii. Secondary Electrospray Ionization Mass Spectrometry
      iii. Selected ion flow (tube) mass spectrometry
      iv. Hallimeter
      v. Breathalyzer
      vi. Gas chromatography-mass spectrometry GC-MS
      vii. Gas chromatography-UV spectrometry GC-UV
      viii. Ion mobility spectrometry IMS
      ix. Fourier transform infrared spectroscopy FTIR
      x. Laser spectrometry Spectroscopy
      xi. Individual chemical sensors or chemical sensor arrays, (e.g., electronic noses)
   b. In vivo analysis of gases (e.g., an ingestible capsule) may be performed with one or more of the following methods, including but not limited to:
      i. Gas sensors modulated by heating elements to selectively respond to certain gases
      ii. Membranes with embedded nanomaterials that allow for fast diffusion of dissolved gases
      iii. Filters that selectively allow only certain gases to permeate
      iv. Diffusion of dissolved gases while efficiently blocking liquid
      v. Tuned semiconductors with a gas profile extraction algorithm
      vi. Oxygen- and/or gas-sensitive polymers
3. Temperature at sample locations may be measured with a wide variety of methods, including but not limited to:
   a. Negative thermal coefficient thermistors
   b. Infrared and/or near-infrared (thermocouples, thermopiles, etc.)
   c. Digital thermal sensors
   d. Temperature-sensitive polymers (cyclododecane, methanesulfonic acid wax, etc.)
4. Pressure at sample locations may be measured with a wide variety of methods, including but not limited to:
   a. Transductive and/or transmission
      i. Strain gauge
      ii. Piezoresistive
      iii. Piezoelectric
      iv. Capacitive
      v. Inferometric
5. Alkalinity (pH) at sample locations may be measured with a wide variety of methods, including but not limited to:
   a. Electrode
      i. Differential sensor
      ii. Combination pH sensor
   b. pH-sensitive polymers (polymethacrylates, enteric elastomer, etc.)
6. Minerals, sample composition and electrolytes
   a. Ion selective electrodes for dietary minerals and electrolytes
   b. Moisture-responsive polyanhydrides
   c. Enzyme-sensitive polymers (chitosan, starch, etc.)
7. Electrochemical
   a. Voltammetry
8. Optical
   a. CMOS imaging sensors
   b. CCD camera
   c. LED (white, fluorescent, etc.)
   d. Optical fibers

### D. Data Storage

For each sample taken, the resulting data, including the corresponding time and location information is stored in one or more electronic storage devices for further processing and analysis.

As with the context factors, note that each of these location and microbiome measurements may also be represented as probabilities. One reason to use probabilistic representations is to account for the possibility of inaccurately measured information (e.g., due to sensor precision), insufficiently sampled information or information that is not current.

### V. User Data

In this section, different types of data that may or may not be associated with a user are detailed. These user context factors, user engagement data, linked subjects, user-subject linkages, coach-user linkages, user-aggregated coach user interaction data and the possible time/location of these data associated with a subject, are collectively referred to as “user data” and each specific type of data as a data “element”. For clarity, the user data for different subjects may or may not contain all the same types of data elements and each user may or may not contain any or all data elements. Note that user data for a certain user may be considered to encompass some or all subject data from one or more subjects that are associated with that user (including subject-subject linkages and subject data for linked subjects) and may or may not also encompass some or all coach data and user-coach interaction data for one or more coaches linked to the user. For clarity, the subject data for different subjects may or may not contain all the same types of data elements and each subject may or may not contain any or all data elements.

Any or all of these user data may or may not be stored on one or more electronic storage devices.

### A. User Context Factors

In order to better serve a user and/or customize the user experience, certain user context factors may or may not be gathered about the user. A variety of potential user context factors to be obtained are contemplated. Some of these context factors can be obtained either via questionnaires for the user or via connection with another system (e.g., a social media account, a hospital account, etc.). These user context factors about the user may include one or more of, but are not limited to, these examples:
1. Age (birthday)
2. Ethnicity or race
3. Any information shared via account linking with another system (e.g., social networks, social media)
4. Gender history and status
5. Sexual orientation
6. Relationship status (e.g., single, married, divorced, in a relationship, etc.)
7. Shopping, search or online data history
8. Languages spoken
9. Occupation
10. Socioeconomic status and history
11. Education status and history
12. Religion or political beliefs
13. Geographic location
14. One or more goals associated with one or more linked subjects
   a. Goal(s) history
   b. Goal(s) status
15. Payment information (credit card, etc.)
16. Contact information (email, phone number, physical address, etc.)
   a. Emergency user contact information

For clarity, any of the example of user predispositions may also be input as user context factors.

### B. User Engagement Data

The user may be interacting with the system in multiple different ways. Some examples of user engagement include but are not limited to:
1. Accessing subject data for one or more subjects
2. Answering new survey questions or quizzes about a user and/or subject(s)
3. Engaging in research
4. Logging diet, fitness, menstrual or other data for one or more subjects
5. Adding or remove linkages
6. Enrolling one or more subjects in clinical trials
7. Posting on message boards
8. Purchasing products
9. Engaging with messages or advertisements
10. Interacting with coaches or medical professionals
11. Communicating with other users linked to the same subject (e.g., a patient (subject) and a doctor communicating through the system)
12. Navigating web pages or mobile apps
13. Reading, searching, listening to or otherwise engaging with educational materials
14. Communicating with other similar users and/or users linked to similar subjects (e.g., providing suggestions, notes, support groups, tips or recommendations to other users)
15. Communicating with other users broadly (e.g., providing product or other recommendations, suggestions, notes or tips to other users)

For clarity, natural language processing (NLP) and/or sentiment analysis may be applied to one or more of these user engagement data elements to add additional user engagement data. For one example, NLP could be applied to audio recording of coach communication to collect data to assess a subject for cognitive decline. As another example, sentiment analysis could be applied to analyze a user (who themselves is the subject) posting on message boards to assess the subject's pain level. Any outputs of NLP or sentiment analysis used on user engagement data is considered as an additional form of user engagement data.

### C. User Aggregated User-Coach Interaction Data

As described below, the user may or may not have interaction with one or more coaches. A combination of one or more user-coach interaction data may or may not be associated with a user. Such a combination may be termed as “user aggregated user-coach interaction data”.

### VI. Coach Data

One or more coaches may or may not be associated with one or more users. For example, a user-subject who wanted to both lose weight and who suffered from diabetes may benefit from having two separate coaches to help them lose weight and manage their diabetes. Similarly, two different users may share a coach.

### A. Coach Context Factors

In order to better serve a user and/or customize the user experience, certain coach context factors may or may not be gathered about the coach. A variety of potential coach context factors to be obtained are contemplated herein. Some of these context factors can be obtained either via questionnaires for the coach or via connection with another system (e.g., a social media account, a hospital account, etc.). These coach context factors about the coach may include, but are not limited to, one or more of the following examples:
1. Age (birthday)
2. Ethnicity or race
3. Any information shared via account linking with another system
4. Gender history and status
5. Sexual orientation
6. Relationship status (e.g., single, married, divorced, in a relationship, etc.)
7. Shopping, search or online data history
8. Languages spoken
9. Occupation
10. Socioeconomic status and history
11. Education status and history
12. Geographic location
13. Goals associated with one or more linked subjects

### B. Coach Aggregated User-Coach Interaction Data

As described below, the user may or may not interact with one or more coaches. A combination of one or more user-coach interaction data may or may not be associated with a coach. Such a combination may be termed as “coach aggregated user-coach interaction data”.

### VII. Data Processing and Analysis

### A. Data Representation for Subjects and Data Elements

In the inventive concepts described herein, each subject is represented in the system by a set of data elements that represents one or more of the contextual information and the microbiome measurements. Each microbiome measurement has a location and/or time associated with it, when available. One or more of these data elements may be missing for any particular subject. When a subject is missing one or more elements of subject data, the system may or may not fill in one or more missing elements via sampling likelihoods (e.g., marginal or conditional probabilities) from the system subject database, taking representative values from published scientific literature, etc. Some subjects may have no contextual information or microbiome measurements, created purely as a function of linkages with other subjects in the system. Each data element may also associate a time with that data element indicating either when the data element was generated (e.g., from a patient measurement or context element) or, if not available, when the entry of the data element into the system occurred. When more recent data elements for a subject are input into the system, these elements are added to the data representation of that subject, potentially with a new time associated, without replacing or deleting the previous data element. All subject representations are stored in one or more electronic storages devices. Note that, from time to time, data from multiple subjects may be merged into one subject or data from one subject may be split into multiple subjects. For example, if a new subject entry was created due to a linkage and it was later determined that this new subject entry corresponded to an existing subject in the system, then these subject entries (accounts) may be merged.

### B. Data Representation for Linkages

All defined linkages are represented on an electronic storage device and may change in response to new data or modified mechanisms for defining linkages. Linkages, weights, signs and directionality may be represented electronically via any number of standard methods, including but not limited to labeling the electronic entry of a subject (e.g., in an array, linked list, object in object-oriented language) with one or more labels representing linkage membership or by storing different types of linkages as groups (e.g., arrays, objects) of subject identifiers. Depending on the representation for linkages, the ordering of subjects in the linkage representation may or may not be considered significant (e.g., to represent directionality and/or sign). Linkages may also be altered over time and, as with subject data changes, these updates are added to the data representation of that subject and linkages, potentially with a new time associated, without replacing or deleting the previous data linkage representation. For example, if two subjects lived in the same household and later changed this living situation, the system may record the new (absence of) linkage between the subjects, while still maintaining a record of the previous linkage.

### C. Linkages to Improve Subject Data Elements

Because linkages represent potential microbiome interactions between subjects, information about one subject may provide additional or improved information about a linked subject. Each sample taken from a subject most often will not contain all elements of the subject's microbiome. Therefore, if a linkage between subjects represents microbial sharing, then a sample about one subject may further inform knowledge about another subject. Estimating a true population (in this case, the microbiome elements of a subject) from one or more samples is a core problem of sampling theory and parameter estimation methodologies. In this case, although the linkage between two subjects represents an incomplete microbial overlap, two samples from two linked subjects provides an improved estimation of the complete microbiome population of each subject than would be obtained by treating each subject sample independently.

The type of linkage can be treated differently in using multiple samples from linked individuals to better estimate microbiome populations. For example, two subjects with a linkage representing a shared geography may have similar microbial populations due to similarities in ambient temperature, humidity, moisture, weather, local microbial environment etc. In contrast, two subjects with a linkage representing a romantic relationship and shared household, may have a more significant overlap in microbial populations, particularly in certain locations (e.g., the skin microbiome due to physical contact).

Linkages may also be used to inform missing information, including context information, about each subject. For example, if one subject is missing geographical location information and this subject is linked to a localized community (e.g., an employer, church, etc.) where each subject in the community is all in one specific geographical location, then there is a high probability that the subject with missing geographical location information is in this same specific geographical location.

In this manner, linkages associated with a subject provide additional information which can be used to better improve and keep current the information of the subject, enabling more accurate data analysis, predictions and user feedback from the system.

### D. Dimensionality Reduction

Dimensionality reduction is a method for reducing the number of variables in a dataset in order to better visualize data and/or as a preprocessor for a dataset prior to machine learning and/or other analysis. Dimensionality reduction may be performed by the following steps:
1. Receive one or more first sets of subject data with k data elements (input data dimension)
2. Optionally receive a number indicating the number of target data elements (less than or equal to k) or other parameter to the dimensionality reduction algorithm to indicate the level of dimensionality reduction to be performed
3. Apply a dimensionality reduction method to the first sets of subject data. A wide range of dimensionality reduction methods can be used, including but not limited to principal components analysis, manifold learning, Laplacian eigenmap, isomap, locally-linear embedding, self-organizing map, t-distributed stochastic neighbor embedding, maximum variance unfolding, etc. Depending on the selection of the dimensionality reduction method, the output may be a second set of subject data with r elements such that r<k and/or a dimensionality reduction system (e.g., a basis) that can be applied to reduce the dimensionality of the first set of subject data and/or subsequent sets of subject data.
4. Optionally output the second set of subject data to an electronic storage device
5. Optionally output the dimensionality reduction system (e.g., basis) to an electronic storage device. This dimensionality reduction system may then be applied to the previous or new first sets of subject data to create a second set of dimensionality-reduced subject data. In this case, the second set of dimensionality-reduced subject data may be displayed to a user and/or stored in an electronic storage device.

Unsupervised and/or generative learning methods (e.g., clustering, k-means, etc.) may also be used for similar purposes. Specifically, clustering subject data may be used as a preprocessor for subsequent methods, to visualize clusters for data discovery or even as a form of dimensionality reduction (e.g., reducing patient data to belonging to one or more of a small set of clusters).

### E. Subject Assessment and Predispositions

In order to inform the user (which may be the subject themselves, parent, doctor or some other relation to the subject) about the state of the subject, this part of the disclosure addresses how the subject data in the system may be used to provide more information for the user. There are many ways in which the data in the system may be used to better inform the user, as subsequently described.

Data/information access for a user includes one or more output devices, which may or may not be electronic (e.g., monitor, laptop, smartphone, paper, electronic document, EMR, etc.).

For clarity, there may be multiple types of users accessing information for one or more subjects and these different types of users may have access to different data elements. For example, a first user may be the subject themselves who is able to access and modify detailed information about themselves in the system. In contrast, a second user may be a researcher who can access only a small portion of de-identified subject data in the aggregate across a population of subjects.

### I. Context Information

One or more elements of the subject and/or user context information may be accessible to the user and may or may not be editable by the user. The context information may be represented as it was input to the system and/or a probabilistic representation of this information may be displayed. Some context information varies with time (e.g., glucose, sleep tracking, nutrition, etc.) and therefore may or may not be visualized for the user with a time-varying plot, graph or display.

When a user is associated with multiple subjects (e.g., a doctor with multiple patient subjects, a farm owner with multiple animal and/or plant subjects), the user may access population-based displays of context information that provides population level statistics, information and comparisons. Context information for one subject may also be displayed to the user in the context of a population, such as all subjects, all linked subjects in the subject's town, all linked subjects in the subject's workplace, all subjects in a certain geography and/or all subjects in a certain demographic range (e.g., males aged 25-35, etc.). The contextual data about a subject in the context of a population could be displayed in multiple ways, e.g., showing where the subject is in a population distribution, whether the subject is above/below mean (medium), etc.

Population data over time may also be displayed in a variety of ways, e.g., as an animation.

### II. Microbiome Information

As described above, our system may represent each microbiota and each biological property of these microbiota and/or their environment (collectively referred to here as microbiome “elements”) as a number indicating the concentration of that element at a particular location. Alternately, other representations could be used, such as a binary number to represent the presence or absence of an element at a particular location or a probability distribution to represent the probability of that element at a particular location. As described above, these locations may be at different physical levels for different subjects depending on the sampling mechanism used (e.g., “GI tract” compared to “duodenum”).

At a most basic level, the user may access and visualize the microbiome representation at one or more locations on and/or within one or more subjects. These visualizations may take a variety of forms, including but not limited to:
1. Raw concentration numbers of each element at each location
2. Pie charts or bar charts showing the distribution of different elements, possibly grouped by genus, phyla or any other taxonomic (or other) grouping. For example, the relative composition of eukaryotes, bacteria and archea and/or fungi, slime molds, protozoa, algae, amoebas, etc.
3. Displayed in the context of location. For example, populations sampled at different locations in the GI tract could be displayed in conjunction with a 2D or 3D display of the GI tract to provide context for each set of population information. This display of the GI tract could either be generic (e.g., overlaid on a display of the GI tract drawn from an anatomy textbook) or personalized to the individual (e.g., based on a radiological image of the patient GI tract, an ingestible capsule display through the GI tract (capsule endoscopy), a surface segmented and 3D reconstructed from a radiological image, etc.).
4. Ontology or hierarchical representation where an ontology is represented along an axis of similarity. As one example, visualizing the phylogenic relationships of subject microbes along an axis based on phylogenic distances. As another example, visualizing the composition of microbes sampled at different locations along an axis of proximity and/or similarity of the locations.
5. Known information and/or studies about the pathogenicity, benefit, interactions and virulence of each of one or more type of microbes
6. Known population of antibiotic resistant genes

For clarity, these concentrations could reflect one or more of any element of the microbiome (microbes, metabolites, virii, temperature, gas concentrations, etc.). As above, these displays could be altered to represent a population of subjects, one subject in the context of a population of subjects and/or evolving through time. For example, a pie chart could represent changes over time with successive pie charts, concentric pie charts where each rim of the pie represents a different point in time, etc. These populations may be defined in many different ways, such as the entire population represented in the system, a population with a certain medical condition, a population located in a certain geography, a population defined by linkages to a subject, a population of subjects associated with the user's account, etc. Various epidemiological information in one or more populations may or may not also be generated and displayed, such as the rate of change for a certain microbe type within a population, across linked individuals or communities, etc.

As described above, the user may be able to access the raw data display of both the contextual and/or the microbiome data. In addition to this information, or instead of it, the user may also have access to additionally processed information generated by the system to provide more descriptive assessments of the subject data. As with all information processing in this disclosure, this processing is done by the system via any of one or more electronic processors (e.g., CPU, GPU, DSP, smartphone, laptop, desktop, cloud, mainframe, etc.).

These descriptive assessments may be calculated separately for one or more types of microbiota and/or including microbiota biological properties and/or environment (such as genetics, proteins, metabolites, transcriptomics, temperature, pressure, etc.) that have been measured or otherwise input into the system for one or more subjects (e.g., bacteria, archaea, eukaryotes, virii, phages, etc.) and/or at certain taxonomic levels (e.g., kingdom, phyla, family, genus, etc.) and/or within a taxonomic level (e.g., firmicutes, bacteroidetes, etc.). Examples of these descriptive assessments include, but are not limited to:
1. Microbiome alpha diversity, which represents the diversity within one population. Alpha diversity may or may not be calculated collectively (all elements of the microbiome) or it may be calculated separately for the different types of microbes. Alpha diversity may be computed at one or more locations at one or more levels of location (e.g., distal small intestine, GI tract, skin, face, whole body, etc.). Alpha diversity may be calculated in many different ways, including but not limited to:
   a. Shannon
   b. Simpson
   c. Hunter-Gaston
   d. Inverse Simpson
   e. Gini-Simpson
   f. Berger-Parker
   g. Any Hill number
2. Microbiome beta diversity, which represents the relative diversity between two populations. Beta diversity may be calculated between many different populations. For example, within one subject, the beta diversity could be calculated between two locations of the same subject (e.g., beta diversity of the upper small intestine compared to the lower small intestine) and/or at two different time points for the same subject at the same location (e.g., beta diversity of the upper small intestine on one day compared to the upper small intestine a week later). Beta diversity may also be calculated between two subjects at the same (similar) location and time or two subjects at different locations and/or times. These two subjects could be chosen in many different ways, such as one user calculating beta diversity between two subjects accessible by the user (e.g., a doctor comparing two patients), between two linked subjects (e.g., a mother comparing her diversity with her child's) or in any other way of identifying two subjects (e.g., randomly). Beta diversity can also be calculated between one subject compared to a population (e.g., of a subject compared to an average of a matched gender population, matched diabetic population, local population, global population) or within a population of multiple subjects (e.g., a farm owner assessing beta diversity of all subject animals and/or plants on the farm). One or more of these beta diversity calculations may be performed holistically for the subjects and/or at certain subject locations and/or times.
3. Rarity of one or more microbiome elements for a subject at one or more locations, compared to one or more of either known databases or the information in the system about one or more populations.
4. Ratio of one or more microbiome elements for a subject at one or more locations, compared to one or more other microbiome elements for a subject (same subject or different subject(s)).
5. Dysbiosis at one or more subject locations
6. Highlighting specific (e.g., pathogenic) species concentrations (or presence) for an individual subject, one or more linked subjects and/or global populations
7. Composite scores of the subject microbiome health
   a. Gut Microbiome Health Index
   b. Immune readiness
   c. Enteric nervous system imbalance

Note that these microbiome diversity calculations may or may not also take into account a similarity weighting between different microbiome elements. For example, in the case of microbes, this weighting could be determined by one or more of phylogenic distance, Faith phylogenetic distance, molecular similarity (e.g., genetic similarity), or even a similarity weighting between two microbes that is learned by machine learning techniques (e.g., based on learning which microbes have similar metabolic effects, etc.). One or more of these assessments may also be displayed to the user to show how these quantities change over time.

Assessments about the sampling and measurement acquisition for one or more subjects at one or more locations may or may not also be displayed to one or more users. Examples of these sorts of assessments include, but are not limited to:
1. Sample information such as sample times, location, processing details (e.g., type of processing, location, vendor, etc.)
2. Sample acquisition information, such as who performed the sample, how long the sample required to take, etc.
3. For certain sample acquisition devices, such as ingestible capsules, the amount of time required to pass through the subject and/or to pass from one subject location to another subject location (residency time). This residency time information could also be used to calculate GI motility and to display to the user.

Any of these assessments could be displayed to one or more users as a function of time or in comparison to a population. For example, comparing a subject's motility (or residency time) from the small intestine to the large intestine to the distribution of motilities from the small intestine to the large intestine of a population (e.g., of similar patients, of the same age range, in a similar geographic location, etc.).

One or more alerts may be created for one or more users (e.g., a person and their doctor) if one or more conditions are met by this data for one or more subjects (e.g., the presence of a significant concentration of a known pathogen in one or more linked subjects). Alerts may also be generated for one or more users associated with a first subject if there are significant changes in the information of one or more linked subjects or if one or more subjects initiates a change in link status with the first subject. These alerts may take one or more different forms, such as push notifications on a smartphone, email, postal mail, web page banners, etc.

### III. Subject Predispositions

The inventive concepts described herein conceptualize connecting together a wide range of subject predispositions (e.g., all medical conditions (physical and mental), traits, behaviors, wellness factors, agricultural outputs, social characteristics, microbiome states, etc.) and subject data to inform the user(s) about risk factors, treatment effectiveness, diet and lifestyle improvement, products and services, etc. related to one or more subjects. Information displayed about one or more subjects to one or more users based on subject data is termed herein as “subject predispositions”. For clarity, these subject predispositions may be determined using all subject data or a subset of subject data. For clarity, these subject predispositions may or may not be determined using only subject context factors (or a subset of subject context factors), only subject microbiome data (or a subset of subject microbiome data elements), only subject linkage information (or a subset of subject linkage information), only subject data associated with one or more linked subjects (or a subset of subject data associated with one or more linked subjects), only user linked data and/or a mixture of these data (or subset of a mixture of these data). For clarity, subject predispositions for different subjects in a single embodiment, or subjects in different embodiments may be determined using different subject data elements, mixtures, etc. For clarity, these subject predispositions may refer to subject predispositions in one or more of the future, present and/or past. A small number of examples of subject predispositions include, but are not limited to:
1. General and miscellaneous
   a. Age
   b. Susceptibility to infection
   c. Colds and cold susceptibility
   d. Infections (ear infections, etc.)
   e. Alcoholism
   f. Smoking (and/or vaping)
   g. Headaches
      i. Migraine
   h. Type of birth (vaginal, c-section, etc.)
   i. Mosquito attractiveness
   j. Missing unsampled biome based on known correlations (e.g., viroids, fungi, etc.)
   k. Subject identity
2. Any or all medical conditions, diseases, disease resistance and disorders (and severity of condition, disease, disease resistance, disorder, etc.)
   a. General
      i. Pain
      ii. Fever
      iii. Metabolic syndrome
      iv. Antibiotic history
      v. Cachexia
      vi. Diabetes (all types)
      vii. Anemia
      viii. Bacterial blooms
      ix. Germline genetics
      x. APOE protein type
      xi. Vitamin deficiencies
      xii. Disease resistance
         1. Malaria resistance
      xiii. Antibacterial resistance to one or more antibiotics at one or more doses
   b. Mortality
      i. Subject death
      ii. Cause of death
      iii. Time of death (past or future)
   c. Oncology
      i. Cancer risk of any type or at any location (e.g., oral, throat, esophageal, gastric, colon, rectal, skin, lymphoma, etc.)
      ii. Cancer recurrence
      iii. Cancer progression
   d. Infection
      i. Bacteremia
      ii. Ear infection
      iii. Parasite presence
   e. Immune disease and inflammatory disorders
      i. Crohn's
      ii. Ulcerative colitis
      iii. Diverticulitis
      iv. Type 1-2 diabetes
      v. Coeliac
      vi. MS
      vii. Lupus
      viii. Rheumatoid arthritis
      ix. graft vs host disease
      x. Inflammation markers (SED, CRP, Rheumatoid factor, etc.)
      xi. Immune function, including functioning of tonsils, adenoids, thymus, spleen, etc.
      xii. IgA deficiency
      xiii. Gout
   f. Heart health
      i. Resting heart rate
      ii. Heart attack
      iii. Stroke
      iv. Atherosclerosis
      v. Blood pressure
      vi. Blood sugar (glucose)
      vii. High serum triglycerides
      viii. Cholesterol
      ix. Heart enlargement
   g. Lung health
      i. Asthma
      ii. COPD
      iii. Lung function
      iv. Breath characteristics (hydrogen, methane)
   h. Liver health
      i. Cirrhosis
      ii. NASH
   i. GI
      i. Colic
      ii. Ulcers
      iii. Small intestinal bacterial overgrowth
      iv. Small intestinal fungal overgrowth
      v. Vomiting
      vi. Heartburn
      vii. Intestinal barrier dysfunction
      viii. Gas
      ix. Celiac disease
      x. GERD
      xi. Diarrhea
      xii. Constipation
      xiii. Bloating
      xiv. Dyspepsia
      xv. Gastritis
      xvi. Indigestion
      xvii. Stool quality
      xviii. Gut motility and residence times
      xix. Cramping
      xx. Leaky gut
      xxi. Irritable bowel syndrome
      xxii. GI events
         1. Colon cleanse
         2. Colonoscopy
         3. Enema
   j. Musculoskeletal
      i. Osteoarthritis
      ii. Joint pain
      iii. Myelopathy
      iv. Morning stiffness
      v. Synovitis
      vi. Bone diseases
      vii. Muscular degeneration
   k. Women's health
      i. Pregnancy
      ii. Morning sickness
      iii. Menstrual cycle
      iv. PMS, cramping and discomfort
      v. Bacterial vaginosis
   l. Neurological
      i. Alzheimer's disease
      ii. Parkinson
      iii. Multiple sclerosis
      iv. Pain tolerance
      v. Mild cognitive impairment
      vi. Dementia
      vii. Memory changes
         1. Memory loss
      viii. Changes in ability to concentrate
   m. Identifying subject eligibility/suitability for a clinical trial and/or study
   n. Recommending clinical, wellness, healthcare services, providers and doctors
3. Treatment response and effectiveness
   a. Efficacy of a particular drug to alleviate a particular condition
      i. Likelihood that a cancer patient will respond to a specific immunotherapy treatment. Likelihood of cancer progression or recurrence
      ii. Efficacy as a function of one or more doses and/or methods of administration
   b. As a biomarker, complementary and/or companion diagnostic
   c. Likelihood that a drug or treatment causes one or more side effects (toxicity)
   d. Contraceptive effectiveness
   e. Immune responses and rejections
      i. Effectiveness of a vaccine for a particular patient
      ii. Organ transplant rejection
   f. Progression of a medical condition or disease in the absence of treatment
   g. Microbiotic treatment effectiveness
      i. Prediction of the likelihood of response to a fecal transplant
      ii. Prediction of subject response to a specific probiotic (containing one or more specific strains) and/or a specific mixture of prebiotic
      iii. Prediction of subject response to a specific probiotic (containing one or more specific strains) and/or a specific mixture of postbiotic
   h. Impact on the subject (e.g., health, wellness, nutrition, microbiome, etc.) of one or more medical procedures, including but not limited to
      i. Antibiotics
      ii. Vaccines
      iii. Fecal transplant
      iv. Colonoscopy
      v. Exposure to radiation
      vi. Colon cleanse
      vii. Surgery
      viii. Hormonal therapy
   i. Impact on the subject (e.g., health, wellness, nutrition, microbiome, etc.) of one or more behavioral or lifestyle changes, including but not limited to
      i. Diet changes
      ii. Vitamin, supplement, prebiotic, postbiotic and probiotic changes
      iii. Geographical location changes
      iv. Fitness and exercise changes
      v. Linkage changes
      vi. Social and sexual changes
      vii. Hormonal changes (e.g., testosterone changes)
4. Microbiome composition
   a. One or more elements of microbiome composition described in section IV. B
   b. Location within or on the subject body of the microbiome composition elements
5. Sensory changes
   i. Vision loss
   ii. Hearing loss
   iii. Loss of taste
   iv. Loss of smell
6. Aging effects
   a. Menopausal effects
   b. Growth and size changes over time
7. Sexual health and behavior
   a. Sex drive
   b. Erectile dysfunction
   c. Fertility and fertility challenges
   d. Urinary tract infection
   e. Sexually transmitted diseases
   f. Subject dating preferences, attraction profile and a good match
   g. Kissing of old and new partners
8. Developmental (also possibly including the risk of onset)
   a. Autism and Asperger
9. Nutritional and fitness
   a. Weight loss/gain
      i. Calorie absorption for one or more different foods
   b. Loss/gain of appetite
   c. Obesity/BMI
   d. Malnutrition
   e. APOE protein type
   f. Kwashiorkor
   g. Food cravings (sugar)
   h. Food intolerances, sensitivities, deficiencies, preferences
   i. Ability to effectively digest different foods, vitamins, minerals, etc.
   j. Subject exercise frequency and type
   k. Exercise impact
   l. Change in diet or food environment
   m. Glycemic response
   n. Individual response to specific food or drink
   o. Nutritional deficiencies
   p. Saccharolytic and proteolytic fermentation
   q. Production of reduced or excess amount of different microbiome outputs (for example: butyrate, lactate, polyamine, phenol, ammonia, hydrogen sulfide, methane, GABA, glutathione, TMA, Propionate, taurine, histamine, indole, estrogen recycling, acetate, zonulin, etc.)
   r. Vitamin biosynthesis (for example: B1 thiamin, B2 riboflavin, B5 pantothenic acid, B6 pyridoxine, B7 biotin, B9 folate, B12 cobalamin, K2 menaquinone, etc.)
   s. Eating pattern (fasting, intermittent fasting, etc.)
   t. Diet (vegan, vegetarian, ketogenic, etc.)
   u. Malabsorption of nutrients (carbohydrates, protein, etc.)
   v. Fat storage
   w. Alcohol response
   x. Caffeine response
10. Allergies
   a. Food allergies (e.g., peanut, shellfish, tree nuts, etc.)
   b. Current versus late onset food allergies
   c. Atopy
   d. Hay fever
   e. IgA, IgE, IgG, IgM
11. Oral health
   a. Periodontitis
   b. Gingivitis
   c. Cavities
   d. Bad breath
   e. Toothache
   f. Caries
   g. Tooth decay
   h. Tonsillitis
   i. Adenoid disease
   j. Oral care (e.g., frequency of toothbrushing, frequency of flossing, etc.)
12. Skin health and beauty
   a. Atopic dermatitis
   b. Acne
   c. Flushing
   d. Wrinkles
   e. Skin softness
   f. Discoloration
   g. Psoriasis
   h. Eczema
   i. Rash
   j. Skin, hair and nail health
      i. Hair loss and/or gain
      ii. Hair thickness and texture
   k. Sun exposure frequency and intensity
   l. Dry skin
      i. Dandruff
13. Mental health
   a. Depression
   b. Anxiety
   c. Mental stress
   d. Mood and mood changes
   e. Malaise
   f. Psychiatric disorders
      i. Bipolar disorder
      ii. Schizophrenia
      iii. Obsessive-compulsive disorder
   g. Brain fog
   h. Mood
   i. Optimism
   j. Energy level
   k. Grit
   l. Fatigue chronic fatigue
   m. Serotonin levels
   n. Sleep quality (e.g., insomnia, etc.)
      i. Optimized time for sleeping and/or waking
   o. Circadian rhythms
   p. Memory loss
   q. Dementia
   r. Cognitive decline
   s. Mild cognitive impairment
14. Time
   a. Expected changes through time, based on current state and history, due to current microbiome composition, week, season, menstrual cycle, etc.
   b. Microbiome stability
15. Family, social and environmental
   a. Heredity or microbiome sharing with relatives, households or social relationships (or other linkages)
   b. Family or social relationships
   c. Local environment, building, living environment, air quality, location, city/town, etc.
   d. Soil samples and geolocation (earth biome project)
   e. Geographic location and/or recent changes
   f. Latitude and geographical location
   g. Humidity
   h. Weather
   i. Temperature
   j. Water, air quality
   k. Pollution levels
   l. Presence, number and type of pet
16. Behavioral, customization and personalization
17. Livestock production, yield, effectiveness, quality, outputs, characteristics, etc.
   a. Milk
   b. Meat
   c. Fur
   d. Leather
   e. Wool
   f. Honey
   g. Work capacity
   h. Organ quality (e.g., for transplantation)
   i. Blood and/or other biofluids
   j. Mating or breeding
   k. Pest resistance
18. Crop production, yield, effectiveness, quality, outputs, characteristics, etc.
   a. Crop density
   b. Pest resistance
   c. Crop yield and quality
19. Cosmetics and beauty products
   a. Positive or negative subject responses and/or interactions to one or more ingredients of cosmetic or beauty products and/or methods of application including but not limited to:
      i. Skin irritation
      ii. Discoloration
      iii. Color changes
      iv. Quality of moisturization
      v. Hair texture changes
      vi. Durability
      vii. Skin softness changes
      viii. Non-viable ingredients comprised of inactivated microorganisms and/or soluble factors (products or metabolic by-products) released by live or inactivated microorganisms, added to a cosmetic product to achieve a cosmetic benefit at the application site, either directly or via an effect on the existing microbiota.
         1. Ferments
         2. Lysates
         3. Extracts
         4. Filtrates
         5. Non-viable microorganisms (inactivated/heat-killed)
         6. Metabolic products or by-products (isolated)
      ix. Non-viable ingredients added to a cosmetic product to be actively used as nutrients by the microbiota (prebiotics) of the application site to achieve a cosmetic benefit.
         1. Fibers
         2. Sugars
         3. Minerals
         4. Complex biological mixtures or extracts
20. Subject preferences and responses to specific products and subjects
   a. Food and drink (e.g., how well will certain food and drink taste to the subject, which food and drink will the subject prefer, mouth feel, etc.)
   b. Beauty and cosmetics
      i. Perfumes (e.g., how well will the subject like certain perfumes or smells, which perfumes and smells will the subject prefer, etc.)
   c. Clothing and materials (e.g., how well will the subject like certain clothes or materials, which clothes or materials will the subject prefer, etc.)
   d. Dating, sexual and/or romantic partners

For clarity, any of the examples of subject context factors may also be produced as subject predispositions, even in cases where the corresponding subject context factors have been entered. In some cases the subject context information is not entered for the subject (e.g., germline genetics) and must be inferred. However, even in examples where the subject data is the same as one or more subject predispositions, it may still be meaningful to display one or more risk scores associated with one or more subject predispositions since the subject data may contain errors, be outdated or there may be a conflict between an element of subject data and a subject disposition which may be useful for the user to be aware of (e.g., knowing that all signs point toward a subject predisposition for the subject to be lean and yet the subject is obese).

Subject predisposition directionality may or may not be additionally determined. For example, if we know that one or more subject data elements are in a state of change, then one or more subject predispositions which depend on that data element may also be in a state of change. Specifically, it has been shown that it is possible to determine which elements of the microbiome are currently in states of significant replication by examining the copy number of microbes. A microbe species where a significant percentage of the population has a large copy number can be determined to be in a state of growth. Therefore, a microbe species in a state of growth may also indicate a direction of change for any subject predispositions that are associated with that microbe.

### i. User Display of Subject Predispositions

One or more subject predispositions may be displayed and/or provided to one or more users and/or coaches in a variety of different ways including, but not limited to:
1. A risk score, over time or at different time point (future or past). Risk scores for each subject disposition may also include:
   a. Flags or otherwise identified elevated risks for the user to be aware of. These flags could be preset by a user (e.g., if a certain risk score goes above a certain level) or automatically by the system (e.g., if a certain risk score is abnormally high for a subject)
   b. Identified groups of users that have an elevated or lower risk (e.g., if the subject belongs to a certain ethnic group with an elevated risk for a certain predisposition, etc.)
   c. Additional information and/or FAQs about each subject predisposition (e.g., information about diabetes, gout, etc.)
   d. Links and/or correlations between different subject predispositions (e.g., comorbidities, etc.)
   e. Positive or negative impacts on predisposition (risk level) as a result of subject changes, including but not limited to diet, fitness, lifestyle, sexual activity, medication, therapies, supplements, geography, water supply, employment, mental wellness (e.g., therapy, meditation, etc.), etc.
   f. References to publications, data, explanations, etc. to inform the user why a subject has the predisposition (risk level) that is displayed and/or why subject changes can cause positive or negative impact on predisposition
2. A visualization of the distribution of subject predispositions for a population and an indication of the subject predisposition in this context. One or more different populations could be used, including but not limited to the population of all subjects in the system, the population of all linked subjects, the population of all subjects having a certain type of linkage (e.g., all subjects in a family, geography, ethnicity, work community, etc.), all subjects meeting certain criteria (e.g., all diabetic females aged 40-50), etc. The visualization could take many different forms, including but not limited to a histogram, pie chart, scatterplot, scatterplot in a dimensionally-reduced coordinate system (e.g., a principal components analysis scatterplot), etc. The indication of the subject in this context could take many forms, including but not limited to arrows, lines, dots, colors, textures, etc.

### F. Determining Subject Predispositions from the Subject Data (Subject Prediction Engine)

Subject predispositions may be determined from the subject data in multiple different ways. Each determined subject predisposition may or may not also have associated a measure of confidence indicating the subject predisposition quality, reliability, evidence, etc. Each determined subject predisposition may or may not also include information about interpretability/explainability or how that subject predisposition was determined from the subject data (e.g., by linking a scientific study). A system that determines subject predispositions based on one or more elements of subject data and/or one or more subject linkages (possibly also including subject data from linked subjects) may be termed as a “subject prediction engine”. As above, the subject prediction engine may or may not also determine one or more confidences, information about the reasoning behind one or more subject predispositions, etc. In this section, we detail many different examples of methods by which a subject prediction engine may be used to determine subject predispositions. For clarity, a subject prediction engine may or may not use one of these methods and/or may combine one or more methods to determine subject predispositions.

### I. Data Transformation and Augmentation

For clarity, the subject data used in any of these methods for a subject prediction engine may be the raw subject data in the system, a transformed set of raw subject data (e.g., normalized, cleaned, dimensionality reduced, etc.), the probabilities/stochastic variables possibly associated with the subject data elements and/or some combination of the above.

In any of the subject prediction engine methods detailed below, the input data and/or output data may or may not be transformed using one or more of many standard techniques. These techniques include, but are not limited to:
1. Data normalization
2. The data used in development (e.g., subject prediction engine training) may or may not also be augmented to reflect properties of the data. For example, if it was believed that some data elements in the subject data followed a Gaussian distribution, augmented development data could be sampled from this distribution. In another example, development data could be augmented by randomly adding and/or removing linkages to the subject to establish stability in the presence of changing social dynamics.
3. The input or output subject data in development and/or deployment may or may not also be replaced by a dimensionality-reduced set of input and/or output data, as detailed above.
4. Sampling and/or cross-validation

For clarity, the inventive concepts described herein conceive that one or more of any data transformation techniques could be applied in different orders in the subject prediction engine.

### II. Based on Publicly Available Information

Many publicly presented studies have been conducted, and more will be conducted in the future, that connect one or more elements of the subject data (e.g., context, associated user data, linkages, microbiome, locations and changes over time) with one or more subject predispositions. As a result of these publicly presented studies, the subject prediction engine may or may not display information to one or more users that connect the results of these studies to one or more subjects (or linked subjects).

In a scenario where this information is being presented to a user, the subject prediction engine may maintain a database or data lake of known connections between various subject data elements and known subject dispositions. This database may represent these connections at one or more levels. For example, if a certain species of bacteria were connected to colon cancer based on a robust set of published scientific studies, then the database could represent this information in one or more ways. For example, a database could represent this information as a connection between that bacteria and colon cancer, between that bacteria at a specific location and colon cancer, between various concentration levels of that bacteria and colon cancer, between that bacteria genus (phyla, etc.) and colon cancer, etc. Each of these representations may also contain information about the confidence in the connection and/or causality (e.g., the bacteria causing colon cancer, the bacteria amplifying severity of colon cancer, colon cancer causing the bacterial growth, mere correlation, etc.). This connection confidence or causality, if included, may be derived from multiple sources, such as the number of published studies showing the connection, the size and quality of those studies, the study population better matching the subject, etc. Thresholds may be defined by the subject prediction engine and/or user such that information about the connection with a subject predisposition is only displayed if the confidence of that connection exceeds the defined threshold. For clarity, the connection between patient data and the patient disposition may be derived from a single element of patient data (e.g., a particular family of bacteria at a particular location in the colon is connected to a subject disposition for colon cancer) and/or multiple elements of subject data (e.g., a certain mixture of subject GI microbes together with certain subject genetics and subject blood biomarker levels are connected to obesity in women). Similarly, subject data about a subject's linkages (or the linked subject data) could also be connected to one or more subject dispositions (e.g., a certain mixture of a first subject's skin microbes together with a certain mixture of skin microbes for a second group of subjects in the set of the first subject's one or more linked romantic partners could be connected to predisposition for increased sexual attractiveness in the first subject). Many different types of subject predispositions could be connected to one or more types of subject data.

The information in such a database could be assembled in many different ways and may be either static or dynamic. Examples include, but are not limited to, one or more of manual entry, a web crawler gathering data from the internet, a publication crawler that mines scientific publications, a software that mines an audio recording of a scientific talk, natural language processing, existing databases, etc. Note that such a database or data lake could be centralized, de-centralized and/or generated dynamically (e.g., a web crawler that searches the web and finds evidence for a connection between subject data and a subject predisposition whenever a connection was displayed to a user).

### III. Microbiome Simulation and Host Interaction

The microbiome is a large, complex, evolving, spatially differentiated ecosystem that interacts with the subject and linked subjects. In order to better understand and make more accurate predictions of microbiome changes and influences, one method for a subject prediction engine is to model and simulate the microbiome, subject and potentially one or more linked subjects.

### i. Microbiome Modeling and Simulation at One Location

Given information about the microbiome from a location, there are multiple methods for performing modeling and simulation of the microbiome at this location by assessing the interaction between elements of the microbiome. Example interactions may include, but are not limited to:
1. Different metabolite productions by some microbe populations
2. Quorum sensing communication between microbes to alter receiving microbe behavior
3. Horizontal gene transfer between microbes, including the likelihood of any gene transfer occurring, the likelihood of specific gene transfer occurring and/or the likely alteration in the receiving microbe due to the horizontal gene transfer
4. Microbe population proliferation and/or reduction in response to changes in the microbiome, location environment (e.g., temperature, pressure, alkalinity, etc.) or nutrient access
5. Impact of fecal transplant from a donor
6. Survival of a newly introduced microbial species
7. Simulation of predator-prey population changes between viruses, phages, subject immune system and/or microbes
   a. Lotka-Volterra simulations
   b. Competitive Lotka-Volterra simulations
   c. Nicholson-Bailey simulations
8. Population growth or decrease of specific microbial populations
   a. Kolmogorov modeling
   b. Population doubling time
   c. Population half life
   d. Demographic processes
   e. Malthusian growth modeling
9. Calculation of steady-state equilibria for an ecosystem
10. Evolutionary game theory calculations
11. Biophysical modeling to model dispersion, transport of microbiome elements
   a. Reaction-diffusion
   b. Diffusion
   c. Transport equations
12. Interactions with the subject or external environment
   a. Response to nutritional changes
   b. Response to the introduction of new species or concentrations (e.g., probiotics, infection, sexual contact, external feces introduction (fecal transplant), etc.)
   c. Impact of antibiotics
   d. Increase (pro-inflammatory) or decrease (anti-inflammatory) in local inflammation
   e. Introduction of a drug
   f. Probiotic introduction
   g. Impact of therapy
   h. Cancer development, tumor formation
13. Flux balance analysis
14. Genome scale metabolic methods

This modeling may be performed categorically (e.g., knowing the rate of metabolite output of a certain microbe in response to a stimulus to model an overall change in metabolite concentration at a location), in one or more spatial dimensions to analyze transient, phase change or steady-state phenomena. The domain and initialization may either be informed and personalized to the subject with measured data (e.g., surface information for a section of the subject's colon, sample measurements, etc.) or a uniform, random or otherwise generic domain and initialization may be used. Data from one or more linked subjects may also be used to inform or personalize the domain and initialization simulation for the subject.

In each of these cases, an initial population must be defined and the change represented by modifications to certain (sub)populations, interactions between (sub)populations, changes to growth (or death) rates for a certain population, spatial interactions, domain changes, boundary condition changes, and the like.

As a non-limiting example of the foregoing, an objective may be to model and calculate the impact of a newly introduced species on the microbiome populations at a location. The concentration of each microbial species (and/or virus, phage, etc.) may be represented by a real number associated with that microbiome element and a real number to represent the newly introduced species. Initial concentrations of each species may be set in accordance with a microbiome measurement at that location, a population (or subpopulation) average, uniformly or randomly. Each species may be assigned an individual baseline death rate over time, a growth rate over time and a rate representing the impact of each pair of species on each other's growth or death rate. These baseline rates and impact rates may established based on one or more of many different factors, but not limited to, the scientific literature, measurements over time, experimental data, other modeling, uniformly and may be fixed, time-dependent or species population dependent.

In this scenario, the impact of a newly introduced species on microbiome populations may be calculated by solving a system of Lotka-Volterra equations either transiently or at steady-state using a computational process. This calculation could be further complexified in many ways, such as by adding terms to represent quorum sensing between microbial populations, changing baseline growth/death rates over time in response to modeled nutritional changes, pH changes, temperature changes or modeling a sudden cross-species drop in population to determine the impact of antibiotic usage in combination with the introduction of a new species.

Another way in which this calculation could be modified is by adding a spatial element to the model to more accurately calculate the impact of a newly introduced species. For example, if the target location was a section of colon, then a generic or personalized spatial surface model of that section of colon could be created (e.g., extracting the subject's individual colon surface from radiological imaging, capsule endoscopy, etc.). In this more complex model, each location in the 3D domain (e.g., a tetrahedral geometric domain representation) of the simulated patient colon could be associated with a real number representing the concentration of each microbiome species at that location (e.g., at each tetrahedron). The initial concentrations of each species at each location may be initialized either via subject sampling information, uniformly, randomly or with any other method. The initial concentration of the newly introduced species could be a single location (e.g., concentration of zero everywhere else), uniformly across tetrahedra or via any other manner. Boundary conditions for the domain may be set using any number of standard methods such as Dirichlet, Neumann, Robin, Helmholtz, Cauchy or a mixed boundary condition. These boundary conditions may be set using subject sampled information or set more generically (e.g., a Dirichlet boundary condition of zero, the population averages, randomly, etc.). Interaction of species concentrations between tetrahedra could be modeled mathematically in many ways, for example as a diffusion relationship (transport equation, etc.) with the Lotka-Volterra equations governing the internal element dynamics. Given this domain, boundary conditions, initialization and coefficients, the transient or steady state may be calculated through a variety of different methods, such as a finite element method (finite difference, etc.).

In this example, multiple different ways in which a microbiome system could be modeled as part of the subject prediction engine were described in order to calculate the impact of the introduction of a new species. This example model could also be used to calculate the impact of a fecal transplant (e.g., by initializing the domain with multiple new species and/or changing the initialization of the concentrations of the existing species) or multiple other of the example models and calculations described above.

The metabolites produced by the ecosystem of species could be further added to the model. For example, each metabolite concentration could be represented as a real number that is a function of one or more species concentrations at a particular location. These functions could be determined from the literature, from experiment, or assumed to follow a standard relationship (e.g., different concentrations are simply proportional to different species). Adding in this metabolite element to the model enables the calculation of a variety of metabolites at different locations over time as species concentrations change. In this example, a decay term for one or more metabolites could be further included to model removal of these metabolites over time (e.g., due to subject bodily consumption, etc.).

### ii. Location Interactions

One aspect of the concepts described herein are that microbiome samples may be acquired at one or more locations at one or more time points. These locations may be precisely defined (e.g., first sample from the small intestine 15.2 mm distal to the pyloric valve compared to a second sample from the small intestine 16.1 mm distal to the pyloric valve) or more categorically defined (e.g., first sample from the oral cavity compared to a second sample from the GI tract). These different locations can be modeled as interacting with each other in multiple different ways. In the previous example above, the two samples could be modeled as coming from adjacent tetrahedral subsections of a domain representing the subject's small intestine where the two subject samples are represented as two separate initial conditions for microbial concentrations spread uniformly across all domain elements in the two sections. In this case, the interactions between the subsections might naturally be modeled the same way as any two adjacent domains (e.g., in the above example, via a diffusion or transport relationship).

However, location interactions may also be modeled in a variety of very different ways. For example, the relationship between microbial concentrations in the oral cavity and the microbial concentrations in the GI tract could be modeled by assuming that a certain number of microbes in the oral cavity are swallowed and pass into the GI tract. A simple version of this model could, again, have two sets of real numbers to represent the microbial populations of each species is each of the oral cavity and the GI tract. At each time point in the calculation, a random selection of microbial populations in the oral cavity representation could be subtracted (swallowing) and those same microbial populations (or a diminished amount or subset) could be added to the population representation in the GI tract to model the swallowing interaction.

### iii. Subject Ecology Modeling

One embodiment of this microbiome simulation and modeling method is to maintain a microbiome simulation model for each subject at one or more locations that is continuously updated as new information is received and/or measured in the subject data. The model may also be updated as more information about specific or general microbe interactions become known (e.g., via scientific publication) to update subject predispositions. Insofar as there are random, stochastic or otherwise probabilistic components of the simulation, more than one sampling of these random components could be used to calculate distributions or probabilities for subject dispositions. Such an ongoing simulation model for each subject could be considered to be a dynamic digital twin of the subject microbiome ecosystem. Such a simulation could be visualized for a user as well, if so desired.

### IV. Machine Learning

As the scientific literature expands, there will be an increasing number of established connections between subject data and one or more subject predispositions that may be displayed to a user. Similarly, simulation methods may also accurately enable calculation of interactions between microbes and their environment to provide estimations of subject predispositions. In addition, a subject prediction engine may also be established using one or more machine learning methods. There are multiple methods that may be employed by a subject prediction engine, that we detail below.

### i. Supervised Learning

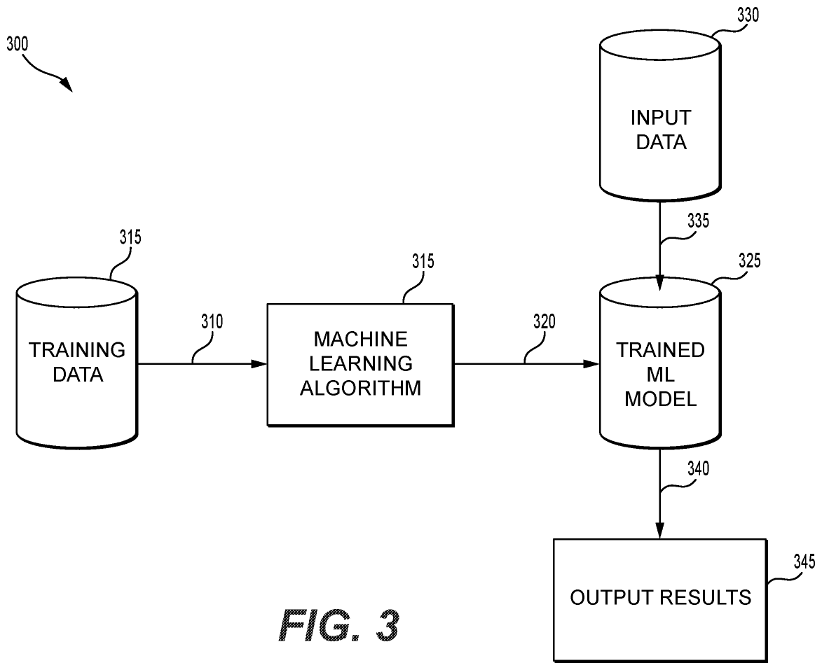

Supervised learning methods may be used by a subject prediction engine to establish a connection between one or more subject data elements, linkages and/or linked subject data elements with one or more subject predispositions. In general, a supervised learning method requires a training and a deployment phase. For instance, referring now to FIG. 3, a flow diagram for training and deploying an exemplary supervised machine learning model is illustrated. The training phase of such a system consists of the following steps:
1. Receive training data 305 that may include labeled associations between subject data (e.g., microbiome sample data, context data, etc.), linkages (e.g., subject-subject linkages, subject-user linkages, etc.), and the corresponding subject predispositions associated therewith.
2. Apply, at 310, the labeled training data 305 to a machine learning algorithm 315 to train the machine learning model to predict one or more subject predispositions from the labeled training data 305.
3. Generate, at 320, a trained machine learning model 325 capable of predicting one or more subject predispositions.

The deployment phase of such a system may consist of the following steps:
1. Receive input data (e.g., one or more sets of subject data, linkages and/or linked subject data) 320.
2. Apply, at 335, the input data 330 to the trained machine learning model 325 to calculate the one or more target subject predispositions
3. Output, at 340, results (e.g., the one or more target subject predispositions) 345 to an electronic storage device, user display, etc.

For clarity, the target subject predispositions may have different types of values, such as categorical (e.g., presence of colon cancer vs absence of colon), ordinal (e.g., colon cancer stage 1, 2, 3 or 4), real-valued (e.g., days to colon cancer recurrence following treatment), etc. Subject data may also have different types of values (e.g., subject weight, presence or absence of a certain bacteria species, etc.) which may or may not be converted to a single type (e.g., real-valued) as needed. A wide variety of supervised machine learning methods may be used in this step, including but not limited to deep learning, support vector machines, k-nearest neighbors, random forests, decision trees, logistic regression, graph machine learning, generative adversarial networks, etc. Different variants of these machine learning methods may be applied to match the type of the target subject predisposition as needed (e.g., regression, classification, etc.) and/or the type of the target subject predispositions may also be converted to another type during training (e.g., converting the target predispositions to real values).

Another possibility would be to follow an unsupervised method (e.g., clustering, k-means, etc.) with a discriminative method (e.g., by using a manual or automated method of identifying a discrimination boundary) in the same manner as a supervised classifier to assign subjects in different clusters to different sets of subject predispositions.

### V. Knowledge Graph

One or more knowledge graphs or knowledge bases could be constructed and updated by mining a variety of different sources, including but not limited to public databases, social media, scientific literature, subject data, user data and coach data. These data may be used to connect together various elements of microbiota and other elements of the microbiome with information including, but not limited to, subject predispositions, user predispositions, human, animal and plant health, nutrition, wellness, etc. A knowledge graph may or may not be manually and/or automatically curated and may leverage natural language processing (e.g., large language models, foundation models, etc.), video analysis, audio analysis, sentiment analysis, etc. technologies. Inferences on a knowledge graph may be used to derive subject predispositions from subject data and may or may not also be used to provide the reasoning, explainability and/or causal chain behind these inferences.

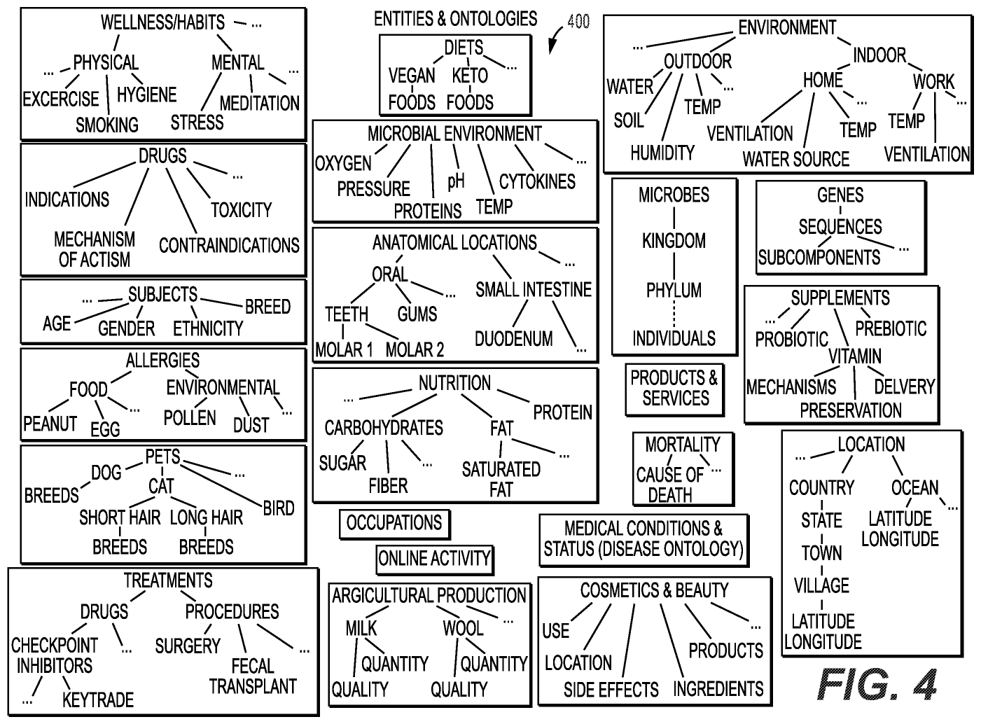

A knowledge graph may include a wide variety of entities and/or ontologies of entities. Any element of subject data, user data and/or coach data may represent an entity and/or be contained in an ontology. Examples of entities and ontologies of entities include, but are not limited to, the examples in the knowledge graph 400 illustrated in FIG. 4. For clarity, the entities and ontologies of entities in a knowledge graph may be static or change over time, via manual changes and/or automated changes. One or more knowledge graphs may or may not additionally include information about data provenance (e.g., justification), attribution, and/or uncertainty assessment of knowledge contained in the graph. For clarity, one or more knowledge graphs may take different forms, including but not limited to a directed edge-labeled graph, property graph, etc. and may or may not contain more complex structures such as hypernodes and/or hyperedges.

A wide variety of connections (i.e., directed and/or undirected edges) may be included in the relationship graph between different entities and/or ontological levels. A small number of examples include, but are not limited to:
1. Subject-subject connection connections
   a. Family member
   b. Sibling
   c. Parent-child
   d. Co-workers
   e. Spouses
   f. Romantic partners
   g. Co-habitants
2. Microbe-microbe connections
   a. Predator
   b. Prey
   c. Communication and sensing
   d. Correlation
3. Microbe-nutrition connections
   a. Microbe eats nutritional component
   b. Nutritional component causes microbe proliferation
   c. Nutritional component causes microbe reduction
4. Microbe-health condition (wellness, allergy, mortality, etc.) connections
   a. Microbe correlated with health condition
   b. Microbe causes worsening health condition
   c. Microbe causes improvement to health condition
   d. Health condition causes microbe proliferation
   e. Health condition causes microbe reduction
5. Microbe-microbial environment (external environment, diet, habit, pet, supplements, occupation, etc.) connections
   a. Microbe correlated with environmental factor
   b. Environmental factor causes microbe proliferation
   c. Environmental factor causes microbe reduction
6. Agricultural output-microbe (diet, drug, external environment, supplements)
   a. Microbe correlated with agricultural output
   b. Microbe causes improved agricultural output
   c. Microbe causes reduced agricultural output
7. Microbe-drug (cosmetic, beauty product, supplement, medical treatment, agricultural production, etc.) connections
   a. Microbe correlated with drug efficacy
   b. Microbe correlated with drug usage
   c. Microbe causes improved drug efficacy
   d. Microbe causes reduced drug efficacy
   e. Drug causes microbe proliferation
   f. Drug causes microbe reduction
8. Drug-health condition (wellness, allergy, morality, etc.) connections
   a. Drug is indicated for health condition
   b. Drug is contraindicated for health condition
9. Health condition-health condition (wellness, allergy, morality, etc.) connections
   a. Comorbidity
   b. Correlation
10. Microbial environment-drug (cosmetic, beauty product, supplement, medical treatment, etc.) connections
   a. Microbial environmental factor is increased with drug
   b. Microbial environmental factor is decreased with drug
   c. Microbial environmental factor is correlated with drug
11. Gene-microbe connections
   a. Microbe associated with gene
12. Microbe-protein (microbial environmental factors, etc.) connections
   a. Microbe produces protein
   b. Protein causes microbe response (e.g., proliferation, reduction, production of additional microbial outputs, etc.)
13. Online activity-health condition (occupation, wellness, pets, location, microbe, environment, etc.)
   a. Online activity correlated with health condition
14. Location-environment
   a. Location correlated with environmental factor
15. Anatomical location-microbial environment
   a. Anatomical location correlated with microbial environmental factor
16. Nutrition-food (drink, diet, etc.)
   a. Food contains certain nutritional component (e.g., in certain concentrations)

For clarity, multiway connections (e.g., hyperedges) similar to the above may or may not also be included. Some examples of multiway connections include but are not limited to:
1. Drug indicated for a certain health condition in a certain geography (e.g., country, etc.)
2. A certain microbe in the subject duodenum causes improved drug efficacy, while the same microbe in the jejunum causes reduced drug efficacy
3. A set of microbes is correlated with a certain medical condition, e.g., a presence of three specific microbes in specific ratios (or absolute quantities, etc.) in the duodenum is correlated with a certain health condition.
4. Certain concentration changes of a certain microbe at multiple time points are correlated with a drug

For clarity, these connections may or may not also account for temporal effects (e.g., the initial introduction of a drug causes microbial proliferation and after two weeks the microbial environment returns to a pre-introduction state, etc.). For clarity, the entities may have one or more of multiple different representations, such as binary, integer, categorical, real valued, etc. (e.g., a drug might be represented as binary (presence or absence) and/or as a real value (dosage quantity over time)).

With these one or more knowledge graphs, the system may include a variety of different methods for searching, querying, reasoning, inference, improvement or other methods for interacting with knowledge from a knowledge graph, including but not limited to schema (e.g., semantic, validating, emergent, quotient graphs, etc.), context methods (e.g., direct representation, reification, higher-arity representation, annotation, contextual knowledge repositories, etc.), deduction, interpretation, property axiom definition, standard ontology operations, classes, semantics, entailment operators, reasoning operations (e.g., rule-based, inference rules, description logics, etc.), induction operators (e.g., numeric, symbolic, unsupervised, self-supervised, supervised, etc.), graph analytics operations (e.g., centrality, community detection, connectivity, node similarity, graph harmonics, graph drawing techniques, graph embeddings, path finding, etc.), machine learning methods (e.g., graph neural networks, recursive graph neural networks, convolutional graph neural networks, symbolic learning, rule mining, axiom mining, etc.). A knowledge graph may also use one or more different identifiers (e.g., persistent identifiers, external identity links, datatypes, lexicalization, existential nodes, etc.) and/or include one or more refinement, quality improvement, correction (e.g., fact validation, inconsistency repair, etc.) and/or completion tools.

### G. User Assessment and Predispositions

In addition to subject predispositions to certain diseases, traits, therapies, etc., the user may or may not also have certain predispositions based on user behavior, personalization, coach interaction, customization, etc. These predispositions may be termed as “user predispositions”. Examples of user predispositions include, but are not limited to:
1. Identifying one or more educational topics that are most likely for the user to engage with and/or benefit from (e.g., most likely to cause a change in behavior)
2. Identifying which coaches a user is most likely to benefit from, engage with, and/or prefer
3. Identifying which products, advertisements and/or services a linked user is most likely to benefit from and/or engage with, purchase, recommend, etc. Some examples may include but are not limited to:
   a. Vitamins, supplements, prebiotics, probiotics, postbiotics
   b. Diets and/or food products and services (e.g., diet and meal plans)
   c. Drinks, shakes, juices
   d. Health products
   e. Oral care
   f. Cosmetics and beauty products
   g. Pet/animal/plant products
   h. Agricultural products
   i. Fitness programs
   j. Coaching or app services
   k. Healthcare providers

For clarity, any of the examples of user context factors may also be produced as user predispositions. These user predispositions may be determined from one or more current and/or historical factors including but not limited to:
1. Subject data for one or more linked subjects linked to the user, subject linkages of those subjects and/or subject data associated with those subjects
2. User engagement data
3. User context data
4. One or more coach-user linkages and/or coach-user interaction data
5. User goal data
6. Any other data associated with a user

### H. Determining User Predispositions from the User Data (User Prediction Engine)

User predispositions may be determined from the user data in multiple different ways. For clarity, recall that user data may or may not include some or all user coach interaction data, some or all subject data for one or more subjects that the user is linked with, information about subject-subject linkages for those subjects and/or some or all subject data for one or more additional subjects linked to those subjects.

Each determined user predisposition may or may not also have associated a measure of confidence indicating the user predisposition quality, reliability, evidence, etc. Each determined user predisposition may or may not also include information about how that user predisposition was determined from the user data (e.g., by showing the user their prior history). A system that determines user predispositions based on one or more elements of user data may be termed as a “user prediction engine”. As above, the user prediction engine may or may not also determine one or more confidences, information about the reasoning behind one or more user predispositions, etc. In this section, different examples of methods by which a user prediction engine may be used to determine user predispositions are detailed. For clarity, a user prediction engine may or may not use one of these methods and/or may combine one or more methods to determine user predispositions.

Similar to the subject prediction engine, the user prediction engine may employ machine learning, supervised learning, neural networks, etc. In addition, prior to use in the training or deployment of a user prediction engine, the user data may or may not be transformed, augmented, etc. similar to the subject data transformation and augmentation described above.

### I. Recommendation Systems

Some user predispositions are likelihoods that a user would be more likely to benefit from, engage with and/or prefer certain educational topics, products, services, advertisements and/or coaches. For these user predispositions, possibly but not necessarily including others, a user prediction engine may use recommender systems.

The training phase of a recommender system may consist of the following steps:
1. Receive one or more target user predispositions to predict (recommend)
2. Receive some or all user data associated with one or more users
3. Use a processor to apply a recommender system training method to train a recommender system that predicts (recommends) the one or more user predispositions from the data

The deployment phase of such a system consists of the following steps:
1. Receive some or all user data associated with one or more users
2. Apply the trained recommender system to calculate the one or more target user predisposition recommendations
3. Output the one or more target user predisposition recommendations to an electronic storage device, user display, etc.

A wide variety of recommendation system methods may be used in this step, including but not limited to collaborative filtering, content-based filtering, reinforcement learning methods, session-based methods, risk-aware systems, etc. As one or more user's data changes (e.g., additional interactions with the system, new coaches, new products, new services, etc.) the recommendations may or may not continue to update.

As a non-limiting example, a system associated with the foregoing concepts may be used to recommend education material for a user. In this example, one user for a subject may be the subject themselves. This user may have set a goal for themselves to reduce acne. By assessing the subject's nutrition, GI microbiome, skin microbiome and environmental humidity, the system may recommend educational materials on the causes of acne, the role of nutrition and the subject's skin microbiome. At a later time point, if the user has read the initial educational material and the acne is becoming less severe, the system may recommend new educational material on related skin conditions, how to improve the skin health and recommend vitamins and skin creams. A second user dermatologist with user access to the subject may be recommended some additional educational materials to remind the dermatologist about related conditions and recommend that the dermatologist follow up with the (user, subject) patient in six months. This recommendation could trigger a reminder for the dermatologist to follow us (see more about alerts below).

### VIII. User Guidance and Interaction

In the previous sections, descriptions were provided regarding how the system could be used to inform the user about the subject data that has been measured or otherwise input into the system. Additionally, descriptions were provided regarding how the system could be used to provide the user with one or more subject predispositions that could indicate to the user a variety of different risk factors, traits, efficacy of different treatments, etc. In this section, descriptions are provided regarding how the system can use the subject data to provide guidance to a user and enable the user to perform interactions that may benefit the user.

### A. Interactive User Assessment of One or More Subjects

The most basic form of user assessment for one or more subjects is via a subject report. Such a report may or may not be static and may or may not include one or more elements such as, but not limited to: Text summary of subject predispositions and/or guidances, visualization of one or more elements of the subject data, a list of one or more subject predispositions (which may or may not include a confidence level and/or predisposition magnitude), a representation of subject and/or user data, related scientific literature (or other publicly available information) and/or a list of one or more subject guidances (which may or may not include a confidence level and/or guidance importance). One or more elements of this subject report may be generated automatically or manually.

The user may also benefit from exploring different scenarios for one or more subjects, to determine the effect of changes to subject data on subject dispositions. For example, a user/subject may want to know how quitting smoking, relocating geography or adopting a vegan diet might affect their microbiome and ultimately their disease risk or weight loss. As another example, a doctor user may want to explore different possible treatment pathways for a patient subject obtained by different diets, medications, etc. As another example, a farm owner user may want to explore the effects of different diets on their livestock subjects.

In order to make these assessments, the system may enable a user to perform the following steps:
1. User identifies one or more subjects to explore
2. System determines a first set of subject predispositions (baseline predispositions) based on the first set of current subject data (baseline subject data)
3. User modifies one or more elements of the baseline subject data to create a second set of modified subject data (hypothetical subject data)
4. System determinates a second set of subject predispositions (hypothetical predispositions) based on the hypothetical subject data
5. System displays the hypothetical predispositions to the user using a display device and/or stores the hypothetical predispositions to an electronic storage device
6. Optionally, the system may show a comparison of the hypothetical predispositions with the baseline predispositions, possibly highlighting areas of significant change. If multiple subjects were selected for both baseline and hypothetical, these comparisons and highlights could be at a population level.

Users may also have other services provided to them, such as:
1. Communicate mechanisms with other users, such as chat, reviews and/or message boards, etc.
2. Additional information about subject predispositions, baselines and distributions
3. Access to a marketplace where a user can view, search, select and/or purchase products and services provided by the system or by 3^{rd} party vendors. Search, filtering, suggestions, purchasing, history, reviews, shipping and/or user recommendations for this marketplace of products and services may also be available to the user. This marketplace may also personalize, suggest, recommend, customize, pre-select, etc. products and services based on one or more of subject data, user data, coach data (or direct inputs) and/or subject/user predispositions.
4. Notification of clinical trials for which one or more linked subjects may be eligible

### B. User Guidance

In the last section, descriptions were provided regarding how the system could enable the user to interactively explore the predicted effects of hypothetical changes to one or more subjects. Another interactive mechanism for a user to explore opportunities for the subject is to establish a target subject predisposition to change (e.g., blood glucose, obesity, etc.) and have the system determine which changes in the subject data to make in order to (most closely) achieve that target. The term “goal” may be utilized to represent these targets.

As described above, the user may have set one or more goals for one or more subjects. For this section, it is assumed that one or more goals have been added to the system by one or more users for one or more subjects. The user may have set a goal (e.g., weight loss, pain reduction, reduce cancer risk, endurance, etc.), a goal may have come from a different user and/or a goal might be input separately. It may be assumed that a goal is structured such that it has a measurable objective defined in terms of a subject predisposition (e.g., in the case of weight loss the measurable objective is weight) and the goal can be described as optimizing the objective function. Note that a goal objective may also be binary (e.g., a goal to avoid recurrence of cancer or maintain current weight), although such binary goals may or may not be rephrased as a real-valued measurable objective by rephrasing the goal in terms of probabilities as needed (e.g., the above goal could be rephrased as minimizing the real-valued probability of cancer recurrence). The term “goal data element” may be used to describe the subject predisposition that the goal is targeting.

In working to achieve the goal, only some of the subject data elements may be modifiable for a particular subject at a particular time. For example, a patient with diabetes who has a goal to lose weight cannot simply modify (cure) their diabetes status, even if curing their diabetes could have a strong impact on achieving their goal. Therefore, in order to have the system provide guidance to achieving a goal, a certain set of subject data elements are first identified to establish the “modifiable data elements” for the system to optimize. This set of modifiable data elements may change over time (e.g., a person in a leg cast may have less control over their fitness today then they will in the future after they heal) and may be established in many ways including being set by a user, a set of users, predefined in the system, etc. Some examples of modifiable data elements include, but are not limited to diet, lifestyle, medication, therapy, cosmetics, fitness, geographical location, pets, linkages, etc.

Although multiple data elements may be modifiable, each data element may be modifiable with a different cost. These costs may represent many different types of cost, including but not limited to monetary costs, ease of modification, time required to modify, likelihood of adherence, risks associated with modification, etc. In one example, a person may find it much easier to modify their diet than their smoking habits. In this case, both diet and smoking habits are modifiable data elements, but the cost of a diet change would be less than the cost of a smoking habit change. In another example, a doctor may want to lower blood glucose levels for a patient. In this case, the modifiable data elements could be different medications, therapies and diet changes and the costs may be monetary costs (or time costs) such that the doctor can optimize the lowest cost (fastest) way of achieving a blood glucose reduction. Therefore, every modifiable data element may be associated with one or more costs. These costs may come from multiple different sources, including but not limited to, user input, price catalogues, known risks, uniform costs, etc. For clarity, these costs may or may not also be nonlinear (e.g., 30 minutes of fitness activity per day for the subject is low cost but 4 hours of fitness activity per day is nearly impossible for the subject and therefore has an extremely high cost).

The goal and progress toward the goal may or may not be represented to one or more users with one or more scores. The score could be the goal itself (e.g., if the goal were to lose weight, the score could be current weight), a normalized version of the goal (e.g., if the goal were to lose 15 pounds, then the score could be a percentage toward achieving that goal), a function of multiple goals (e.g., losing weight and sleeping longer) or a purely invented gamified score to help the user achieve their goal. Different users may have different scores. Scores may be displayed and/or may be accessible on one or more user interfaces or devices (e.g., webpage, mobile device, screen, etc.).

Mathematically, the user guidance to achieve a goal may be represented as optimizing a function of goal achievement (e.g., the squared difference of the current goal data element to the target goal data element), modifiable data elements and costs associated with changes in these modifiable data elements. Depending on the goal, modifiable data elements and costs, the optimization of this function may be performed with different methods. In the next sections, descriptions of multiple methods that could be used to optimize the function are described. Before doing so, however, the general steps involved in user guidance are described, which may include:
1. Receive a target subject and subject data
2. Receive a target goal for the subject, a set of modifiable data elements and costs associated with modification to each data element
3. The system determines a second set of guidance data elements for one or more of the modifiable data elements
4. The system displays this second set of guidance data elements to one or more users and/or stores these guidance data elements in an electronic storage device

Note that the above steps may be run in a loop as desired, updating subject data, costs, goals, modifiable data elements, etc.

### C. Determining User Guidance

Determining a second set of guidance in the above system may be accomplished via many different methods. In this section, several examples of methods to enable this step are provided which are not intended to be exhaustive.

### I. Based on Publicly Available Information

User guidance may be based in part or entirely on publicly available information. Specifically, user guidance may be based on clinical recommendations, drug warnings, comorbidities of health conditions, nutrition information and/or the scientific literature. For example, a subject with Crohn's Disease may be recommended to avoid alcohol in accordance with clinical guidelines.

User guidance may also be generated by associating instances in the scientific literature where the subject data linked to certain subject predispositions to other instances in the scientific literature where certain actions have been associated with changes in the subject data that would yield more favorable subject predispositions. For example, low levels of E. rectale, F. prausnitzii and high levels of E. coli have been associated in certain scientific papers with an elevated predisposition for Inflammatory Bowel Disease. A different set of scientific papers have associated a ketogenic diet with low levels of E. rectale and high levels of E. coli. Other scientific papers have also linked aspirin with an increase in E. coli. Therefore, the user guidance may be determined from these scientific papers by guiding the user that the subject avoid a ketogenic diet or taking aspirin because these actions could increase the subject predisposition for Inflammatory Bowel Disease (and avoiding these actions may lower the subject predisposition for Inflammatory Bowel Disease). However, other scientific papers have linked a low-fat diet with increased levels of F. prausnitzii. Therefore, the user guidance may be determined that the subject is recommended to adopt a low-fat diet as a method of improving the subject predisposition to Inflammatory Bowel Disease. Such a system could be automated and scaled via inference on a knowledge graph that is derived from the literature (as discussed in proceeding and following sections).

### II. Gradient Descent Type Methods

The ability to assess hypothetical subject predispositions in response to changes in hypothetical subject data enables the ability to perform a gradient descent-like approach to user guidance. One example is:
1. Receive an initial set of subject data, goal, set of modifiable data elements and costs associated with modification to each data element
2. Initialize a current subject data to the initial set of subject data
3. Initialize a current cost to zero
4. Initialize a current goal data element to the initial goal data element
5. Initialize a current objective value to some measure of difference (e.g., absolute value of difference) between the initial goal data element value and the target goal data element value
6. For some number of loops
   a. The system may randomly (uniformly, systematically) perturb one or more of the modifiable current subject data element values to create a set of adjusted modifiable data element values
   b. Create a second set of perturbed subject data that has values equal to the current subject data values, with the adjusted modifiable data element values replacing the modifiable data element values
   c. The system determines one or more perturbed hypothetical subject predispositions based on the perturbed subject data
   d. Create a perturbed current objective value based on the measure of difference between the perturbed goal data element value and the target goal data element value
   e. Calculate a perturbed cost based on the costs associated with modification of each of the current modifiable data elements to the perturbed modifiable data elements
   f. Select the perturbed modified data elements that optimize a combination of the perturbed current objective value and the perturbed cost. This combination may be calculated in multiple ways. One example is by multiplying together the current objective value and the perturbed cost.
7. Display the selected perturbed modified data elements to one or more users and/or store the perturbed modified data elements on one or more electronic storage devices
8. Optionally, display the perturbed hypothetical subject predispositions to one or more users and/or store the perturbed hypothetical subject predispositions on one or more electronic storage devices

These perturbed modified data elements define the guidance to a user at that time. For example, the system may determine through this method that adoption of a ketogenic diet and drinking more water daily will have the most significant (and lowest cost) impact on the subject's weight loss goal. The system may also display to the user the hypothetical subject weight after making these changes as a motivational tool. As the subject adopts these changes and the subject's current subject data is updated, the guidance system may be run again to generate a new set of changes (guidance) for the user to implement to keep optimizing toward their goal.

### III. Reinforcement Learning

Reinforcement learning is another category of machine learning that can be applied to help provide guidance to users and related individuals, such as coaches, to help a subject achieve a goal. Reinforcement learning has proven extraordinarily effective in teaching AI to excel at game-playing and is generally framed as a method to teach a computer to achieve a complex goal (e.g., winning a game of chess) via a series of actions and reactions (moves). In our case, reinforcement learning can be used to teach the computer to make moves (i.e., guide the user to act) in order to achieve a goal (i.e., the user goal, while minimizing action costs).

The user guidance problem may be mapped into the framework of reinforcement learning (e.g., Markov Decision Process) by associating:
1. Agent states with current and/or hypothetical objective values
2. Agent actions as modifications to the modifiable data elements
3. Probabilities associated with agent actions are associated with the costs to modifications in the modifiable data elements
4. Reward for transition is calculated by first using the system ability to generate hypothetical subject predispositions based on modifications to the modifiable data elements and then determining a change in the objective values from the previous state to the new state

Given this mapping to a reinforcement learning framework, a wide range of reinforcement learning techniques may be applied to provide the user guidance toward a goal including but not limited to Q-learning, SARSA, deep deterministic policy gradient, proximal policy optimization, etc. Reinforcement learning systems could be trained in a variety of ways, including training on historical subject data changes, simulation of users/subjects, etc. At each step of a reinforcement learning system used for guidance, the system may output a suggested set of modifications for the user to make in order to achieve their goal. This usage of a reinforcement learning system for guidance is similar to how a reinforcement learning system trained to play chess could make move suggestions for a human player at every turn in the game.

### IV. Knowledge Graph

Inference on a knowledge graph may also be used to determine the most significant modifiable variables impacting a goal data element and how changes in the modifiable variables could impact the goal data element.

### D. Coaching

In order to help a user achieve their goals, a user may have the option to interact with one or more coaches. The coaches and users may communicate through various means, such as written communication, voice, video, alerts, etc. Each coach associated with one or more users is represented as a coach-user linkage which is stored on one or more electronic storage devices. A coach with a coach-user-linkage to a user may or may not have access to one or more elements of the user data (e.g., user engagement data, user goals, etc.) and may or may not have access to one or more elements of data that a user has access to (e.g., subject data for subjects that the user is linked, etc.).

One or more coaches may be matched with a user in many different ways. For example, a user could be provided information on one or more coaches (e.g., coach expertise, coach gender, coach background, etc.) and given the opportunity to select a coach. As another example, a coach may be matched automatically to a user using one of many matching algorithms including, but not limited to, the Hoperaft-Karp algorithm, Edmonds' blossom algorithm, greedy algorithms, online bipartite graph matching algorithms, etc. Additionally or alternatively, as another example, a quality measure of match for a user to a coach could be determined via some means (e.g., demographic similarity of user and coach, a measure of similarity based on coach data and user data, net promoter score of user for a coach, the success of a coach in helping one or more other similarly situated users in achieving or progressing toward their goal, any combination thereof, etc.) and this quality measure could be used either as an edge weighting in a matching algorithm and/or used as a measure from which to train a machine learning algorithm to determine the optimal coach for a user, for a machine learning algorithm to learn an edge weighting, etc.

These coaches may also be provided with additional information which can support their coaching effectiveness for different users. For example, a coach may also be given access to the interactive subject assessment capabilities described above that would enable the coach to explore different scenarios of changes to subject data and the effectiveness of those changes to subject predispositions. As another example, the coach may also have access to the guidance information that the user may have from the system and the ability to identify new goals or change goals and determine the resulting change in guidance information. The coach may or may not also have access to user predispositions and/or any information the user has access to (e.g., subject data).

The coaches may also have access to further information which can help a coach support a user, such as access to data about populations of users, populations of subjects, trends, coaching effectiveness, subject linkages, subsets of subject and/or users (e.g., possibly defined by a coach), etc. As one example, a coach may look for trends in subject linkages (e.g., increase in a pathogen among those subjects in the same family as a subject or sharing the same water supply as a subject), trends in all subjects linked to a user (e.g., for a doctor user, supporting the doctor by identifying trends in the doctor's subject patients) or look to compare a subject to other subjects from a similar population (e.g., to assess how typical a subject is compared to other subjects with a similar medical condition or demographic information, etc.). Note that any or all information the system supplies to a coach could be supplied directly to one or more users either in addition to a coach or instead of supplying the information to a coach.

Interactions between one or more coaches and one or more users may also be analyzed for many reasons, including to improve the user experience, coach effectiveness, etc. As described, the coach-user interactions may take many different forms, such as written communication, phone, video, etc. Each of these interactions may be analyzed using one or more different types of analysis system such as natural language processing (NLP), sentiment analysis, video analysis, etc. to generate data that describes the interactions and effectiveness of these interactions. The data produced by this analysis may be termed as “coach interaction data”. For example, coach interaction data may show that when a user is not progressing toward achieving their goal, that certain coaching techniques, suggestions, attitudes, etc. are more effective than others for helping the user achieve their goal. As another example, the coach interaction data may show that when a certain user expresses frustration, that the most effective response for a coach is to respond in a caring way. As another example, coach interaction data may show that certain coaches had a more positive attitude in general and others had a more skeptical attitude, which could be used as data to better train a system to match coaches to users more effectively. As another example, the coach interaction data may also be used to suggest certain educational materials for the user. Coaches may also enter data about a user or user interaction prior or subsequent to user interaction to record notes about the user or interaction. These notes could be structured or unstructured data, written, spoken, etc. Any such note data is considered as part of user coach data. Coach user interaction data may be associated with a coach user linkage, with the user and/or with the coach. Similarly, user coach interaction data for all users associated with a coach may or may not be combined into a larger set of data associated with the coach. This combination of coaching data may be termed as “coach aggregated user coach data” for a coach. Similarly, user coach interaction data for all coaches associated with a user may or may not be combined into a larger set of data associated with the user. This combination of coaching data may be termed as “user aggregated user coach data” for a user.

Any coach interaction data and/or coach aggregated user coach data could be used to train and deploy a machine learning system to determine many different types of output to improve a coach. One example is to train and deploy a machine learning system that uses the coach interaction data to provide information to the coaches (in real time during a session to respond to a user, prior to a user session, as feedback after a user session, etc.) to give feedback to the coach and enable the coach to be more effective with helping the users achieve their goals. Another example is to use a machine learning system trained on the coach interaction data (and/or aggregate coach data) to determine that certain coaches are being generally less effective than their peers (or less effective with certain types of users) and to identify those coaches for additional instruction.

Similarly, any coach interaction data and/or user aggregated user coach data could be used to train and deploy a machine learning system to determine many different types of output to improve ways a coach can most effectively work with a user. For example, one user may prefer brief, fact-heavy interactions from their one or more coaches while another user may primarily respond to encouragement from their coaches. This information could be used to help coaches tailor their approach for a particular user, and/or improve matching of a coach to a particular user.

Note that coaches may or may not have a coach-specific (web, mobile, etc.) interface. The coach may also be augmented, supplemented and/or replaced for one or more user interactions with an automated coach system such as a chatbot, conversational AI, generative AI, etc. Some examples of topics that the coach may support a user on include, but are not limited to:
1. Effecting changes in one or more subject and/or user predispositions
2. Nutrition
   a. Diet, meal plans
   b. Supplements
   c. Eating patterns (e.g., fasting)
   d. Optimal times to eat certain foods and/or overall optimal eating schedule
3. Fitness and physical wellness
   a. Strength
   b. Flexibility
   c. Endurance
4. Medical issues and/or specialty care
5. Dental and/or oral health
6. Agriculture
7. Livestock
8. Fishery
9. Veterinary
10. Business and/or economics
11. Public health
12. Mental health and wellness (therapy, psychology, psychiatry, focus on a specific issue, specialists in therapeutic techniques, meditation, etc.)
13. Dating and romance
14. Sleep
   a. Optimal time to sleep and/or wake
15. Education
16. Cosmetics and/or beauty
17. Treatment efficacy
   a. Drug treatments
   b. Fecal transplants
   c. Antibiotics
18. Skin health
   a. Sun exposure
19. Forensics
20. Products and services
21. Legal
22. Research
   a. Study and/or trial design
23. Scientific
24. Pets

### I. Generative AI

One or more foundation models could be used to provide guidance to coaches (and/or replace coaches) and related individuals. For example, a generative model could observe coaching sessions to determine a generative model of the coaching session and coach interaction.

### II. Reinforcement Learning

Reinforcement learning is another category of machine learning that can be applied to help provide guidance to coaches and related individuals, such as coaches to help a subject achieve a goal. Reinforcement learning has proven extraordinarily effective in teaching AI to excel at game-playing and is generally framed as a method to teach a computer to achieve a complex goal (e.g., winning a game of chess) via a series of actions and reactions (moves). In our case, reinforcement learning can be used to teach the computer to make moves (i.e., guide the user to act) in order to achieve a goal (i.e., the user goal, while minimizing action costs).

The user guidance problem may be mapped for coaches into the framework of reinforcement learning (e.g., Markov Decision Process) by associating:
1. Agent states with current and/or hypothetical objective values for the user
2. Agent actions for the coach could take multiple forms, including but not limited to possible suggestions the coach might make for the user, reactions to the user, etc.
3. Probabilities associated with agent actions could be assigned in multiple ways, including as uniform probabilities, length of time required for an agent to make a suggestion, etc.
4. Reward for transition could be calculated multiple ways, such as by determining the likelihood of a positive reaction by the user, the likelihood that the user goals (objective values) are improved, generating hypothetical user predispositions based on hypothetical modifications to the user data elements etc. These likelihoods could be determined in many different ways, such as with the user prediction engine, by based on previous coach-user data from one or more users, etc.

Given this mapping to a reinforcement learning framework, a wide range of reinforcement learning techniques may be applied to provide the user guidance toward a goal including but not limited to Q-learning, SARSA, deep deterministic policy gradient, proximal policy optimization, etc. Reinforcement learning systems could be trained in a variety of ways, including training on historical subject data changes, simulation of users/subjects, etc. At each step of a reinforcement learning system used for guidance, the system may output a suggested set of modifications for the coach to make in order to achieve their goal. This usage of a reinforcement learning system for guidance is similar to how a reinforcement learning system trained to play chess could make move suggestions for a human player at every turn in the game.

### E. Alerts

A user and/or coach may also receive one or more alerts in one or more different circumstances. For example, a coach may trigger a user alert or a user may trigger a coach alert. As other examples, a user alert may be generated automatically if a subject exhibits a known pathogen, if a subject is eligible for a clinical trial, if there is a substantial change in user or subject data, if a user's goals for a subject aren't being met after a certain period of time, if a subject is starting to exhibit signs of disease onset (e.g., pre-diabetic), if subject linkages start exhibiting signs of disease onset, to indicate the introduction of new educational material, to indicate that new services are available, etc. The user and/or coach may or may not also establish conditions that would trigger an alert for themselves.

Alerts may take many forms, including but not limited to: mobile device push notifications, emails, physical mail, website banner notifications, etc. In an embodiment, alerts may be visual (e.g., a push notification displayed on a display screen of a user's device, etc.), auditory (e.g., an audible alert emitted from a speaker of a user's device, etc.), haptic (e.g., a vibration alert, etc.), any combination of the foregoing, and the like.

### IX Generative AI

Generative AI techniques may be used to generate multiple aspects of the system, including one or more elements of the subject data, one or more elements of the user data, one or more elements of the coach data, one or more elements of the user interaction and/or one or more elements of the user guidance. For example, a Generative AI system could be used to generate a microbiome population for one or more hypothetical subjects. Another example would be to use a Generative AI system to produce one or more elements of the user guidance or report. Another example of a Generative AI system would be to generate possible future states of one or more elements of subject data, one or more elements of user data and/or one or more elements of coach data. Such a system could be trained by observing one or more subjects, changes to modifiable variables (e.g., lifestyle changes, medications, dietary plans, diagnostics, treatment plans, etc.) and the impacts to other subject data elements over time. Generative AI systems could be trained with any number of methodologies, including but not limited to, Generative Adversarial Networks (GANs), Transformers, and/or Variational Auto-Encoders. Any of the exemplary techniques described below could also be used for training a Generative AI model.

### A. Foundation Models

One or more foundation models could be built to model subject data, subject predispositions and the evolution of subject data over time. For example, one or more foundation models could be trained by one or more of these methods:
1. Training a system to complete one or more sections of scientific papers given the contextual language of the papers
2. Training a system to complete one or more subject or user predispositions, given other subject or user predispositions and/or context factors
3. Training a system to complete one or more transcripts of a coaching interaction given contextual factors or elements of the interaction
4. Training a system to complete elements of subject, user and/or coach data given other elements of subject, user and/or coach data

### X. Exemplary Embodiments

In this section, examples of different scenarios for the inventive concepts are described herein.

### A. Healthcare Provider

In this embodiment, a scenario is described in which a hospital, health system, government or employer has adopted the inventive concepts described herein in order to provide better care for its patients. For simplicity, the term “hospital” is utilized in the description of this embodiment to refer to any of these entities. For this scenario, the following associations can be made:
1. Subjects—Each patient in the hospital is a subject
2. Subject-subject linkages—Patients who are in a shared living situation, patients who live in close geographical proximity
3. Users—Each patient may have multiple users associated with that patient, such as:
   a. The patient themselves
   b. The doctors and nurses on the patient's care team
   c. Hospital researcher
   d. Population researcher
   e. Medical insurance account manager for the patient
   f. Caregivers (e.g., a nursing home, hospice care professionals, etc.)
   g. Family members
4. Coaches—Different users may have access to one or more coaches, such as
   a. Patient and family members—Coaches for a patient or family member could work with the user to help minimize pain, increase mobility, encourage adherence to medication, nutritionist, etc.
   b. Doctors and nurses—Coaches for the care team could be medical specialists (e.g., other doctors or nurses) from another hospital that may provide occasional consultation on key care questions
   c. Caregivers—Coaches for the caregiver could be a medical specialist to help answer questions about optimal care for the patient

In this embodiment, the hospital may create subject and user accounts for each patient. In this case, the subject context factors could be initialized through one or more of a variety of factors, including the hospital data for each patient (e.g., EMR, PACS, LIS, etc.), survey questions from the various users about the subject, insurance data for the subject, social media accounts, etc. Subject-subject linkages could be initialized via user input and/or using the subject data about living situations, address, etc. User context factors could be initialized for each user type in one or more of multiple ways, including user questionnaires, hospital accounts, professional profile information, social media, online purchase history, etc. Following initialization, all of this data could be kept current by propagating changes to the hospital data for a patient, additional survey questions, social media updates, etc.

In this scenario, prior to initiating the program, the hospital performs full body imaging of each patient (e.g., MRI, CT, etc.). A digital anatomical model of each patient is then created from these full body images by segmenting out each anatomical structure (including body surface) to create a digital volume and surface model for all anatomical structures for each patient.

The hospital may collect microbiome data for these subjects through many different means, such as:
1. Regular collection of each patient's fecal samples, blood samples, urine samples, saliva samples, oral swabs, vaginal samples and respiratory samples for testing in a central hospital laboratory and/or external laboratory. Each of these samples being associated with the time of acquisition and location of sample (e.g., “GI tract”, “oral”, “bladder”, “lungs”, etc.).
2. Regular collection of skin swabs, vaginal and oral swabs from multiple locations on the patient skin and oral cavity for testing in a central hospital laboratory and/or external laboratory. Each of these samples being associated with the time of acquisition. The location of these swabs could be selected on a display of the digital model for the patient to match the location of the collected swab. The system records these sample locations and associates with the location the microbiome information obtained from the swab samples.
3. Tethered biopsies and/or scopes (endoscope, colonoscope, etc.) to acquire fecal and fluid samples at specific locations in the patient, which are recorded for both time and location in the GI tract (e.g., sample collected in colon at 5.5 mm proximal to rectum, etc.). These sample locations may be further associated with digital anatomical model of the patient either via calibrated radio beacons on the scopes that are linked to the models, user selection of a sample location on a display of the model, automated reading of the sample location description (e.g., “5.5 mm proximal to the rectum”) and association with the digital model, etc.

Recall that any other healthcare data associated with the patient is included in the subject data. Some examples:
1. Images of the patient produced by radiology, cytology, pathology, optically, etc. could be digitized and analyzed for digital biomarkers, measurement, assessment, anatomical quantification, morphology, radiomic features, pathomic features, etc. Any cells, tissue, skin, etc. may be stained with one or more assays and/or treatments prior to or during imaging.
2. Genetic, genomic (germline and somatic), transcriptomic, proteomic, metabolomic, exposome and other -omic data of the patient, lesions, cancerous tissue, etc.

A subject prediction engine using this data could be developed in one or more different ways, such as:
1. A knowledge graph that supports linking of one or more of these elements together
   a. Microbiome and/or subject context factors
   b. Subject predispositions
   c. Drug interactions (efficacy, side effects, toxicity, etc.)
   d. Nutrition and/or lifestyle
2. A machine learning method (e.g., deep learning, etc.) that is trained with the current and historical state of subject data (including linkages and linked subject data) and current and historical subject predispositions. This machine learning method could be updated as new data becomes available and may be deployed to provide a current set of subject predispositions based on the current and historical subject data (including subject linkage data). For example, by having the machine learning system be trained with multiple examples of patients who have or develop inflammatory bowel disease, the system may learn that certain conditions in the patient microbiomes (e.g., microbe composition and location, time evolution of species, etc.) are highly predictive of the onset of inflammatory bowel disease. Similarly, by being trained on subject data of multiple patients who received and responded differently to a treatment, the subject prediction engine may learn to predict which patients will respond best to which treatment.
3. A biological simulation that accounts for known or hypothesized interactions between different microbe populations (e.g., predator-prey, quorum sensing, etc.) and/or the microbiome environmental factors to determine microbiome and patient changes in over time. The availability of a spatial anatomical digital model of the patient further allows this simulation to have a spatial and temporal component (i.e., by using the location and time information associated with samples) to improve the accuracy of the simulation.
4. As new samples and subject data changes over time, each of these methods for the subject prediction engine may be updated and improved to further enhance their accuracy. For example, if a simulation predicts a certain change in the patient that does not agree with subsequently measured data about the patient, then the parameters of that patient simulation may be refined to improve the agreement between the simulation and data historically and/or for a future version of the subject prediction engine.

Subsequently contemplated are how different users may interact with and benefit from this system.

### I. Patients as Users

As a user, each subject may access their account and receive a range of information about themselves and guidance. The user may access their account in one or more of many ways, such as via a mobile device (e.g., tablet, smartphone, etc.), web interface, hospital terminal, etc. This patient could use the prediction engine to understand their risk profile for various conditions, explore additional information about these conditions and how their subject data is related to their subject predispositions. Further, if the patient inputs a goal (e.g., weight loss, pain reduction, etc.) the system may identify suggested changes for the patient. The patient may also use the subject prediction engine to explore one or more of multiple different potential changes they could make, such as dietary, supplements, lifestyle (e.g., smoking cessation), treatment, sexual activity, etc. to see how these changes would be reflected in changes to subject predispositions.

For example, through this exploration and/or system recommendations, the patient may identify that a dietary change, addition of supplements and a medication change is predicted to substantially reduce their pain level. Having identified this possibility, the patient may use the system to alert their primary care doctor with these changes in order to ask the doctor's opinion. The doctor, as a separate user, may access the patient's data, examine the evidence and suggest back to the patient that the patient tries these changes. The patient user may shop and purchase in the system marketplace for supplements, medications and/or meal plans to help them with these changes. In order to help the patient, the system may provide recommendations (e.g., via a user prediction engine), and/or access to user reviews, message board access, etc. to help the patient decide what is best for them. This patient may also select (or be matched to) a coach who can help them achieve their goal of pain reduction and adherence to these changes. This coach could have access to the same information about the patient, patient-doctor interaction, current pain level and the patient's plan for changes. Through coaching sessions, updates to the patient's subject data, coach recommendations and pain tracking the coach can help the patient with the changes and successfully reduce or eliminate pain.

The system may also use the patient data to send the patient one or more alerts, such as the eligibility of the patient for a clinical trial.

Family members and/or caregivers may be users instead of or in addition to the patient and have access to all or a subset of the patient data in order to help provide better care for the patient. Similar to the patient, these users may examine subject predispositions and/or use the subject prediction engine to explore the impact of different changes on subject predisposition. These users may also set goals for the patient and receive guidance and/or recommendations from the system. These users may also access the marketplace, receive product/service recommendations, access message boards, communicate with the patient's doctors, receive coaching, establish alerts, etc.

The patient may collect subject context data about themselves via any one or more of the methods described in Section V.A. For example, the patient may fill out survey data, link their phone (with usage and sensor data) to the system and either have and/or be prescribed one or more of the home health devices which would be linked to the system (e.g., via Bluetooth, Wi-Fi, etc.). The data from these devices may or may not be calibrated to the individual subject by accounting for other subject context factors and/or subject microbiome data. For example, variations in barometric pressure could be normalized by altitude or frequency of smartphone app usage could be stratified by age.

### II. Doctors as Users

A doctor may access their system account to monitor or interact with one or more patients. The doctor may look to see how changes in treatment are affecting the patient.

As an example, a doctor may notice or receive an alert that a patient with inflammatory disease is getting worse (e.g., by increases in inflammatory markers such as CRP). As a result, the doctor may use the subject prediction engine to explore different treatment and/or dietary and lifestyle choices. Through the use of the subject prediction engine, or a recommendation from the user prediction engine or guidance, the doctor may identify that a certain treatment that the doctor is unfamiliar with is likely to benefit the patient by substantially reducing the predicted inflammation markers. The doctor may connect with a coach, in this case the coach being a doctor specialist at another hospital, to discuss the subject prediction engine results and the use of this treatment for the patient. After the coaching session with the specialist, the doctor may alert the patient through the system that the doctor is changing the patient's medication and then use the system to monitor the patient improvement. The doctor may also set a goal for the patient for adherence to this new medication and suggest a coach for the patient that can help the patient with adherence.

As another example, a doctor may be concerned about a cancer patient who is not responding to the first line treatment. The doctor may use the system to assess whether a new immunotherapy treatment might be more effective for the patient. When the doctor uses a hypothesis of this new treatment for the patient, the subject prediction engine determines a high likelihood for severe side effects of the potential treatment for the patient. However, based on the subject data and connection to a clinical trial database the system may identify that not only is the patient eligible for a clinical trial for a new therapy but, via a knowledge graph, that the mechanism of action in the new therapy creates a higher predisposition of treatment success for this patient. As result, the doctor is able to alert the patient to this new trial and get the patient enrolled.

Another example is for the doctor to use the system as a biomarker, precondition, complementary and/or companion diagnostic for use of a drug or therapy prior to administering the drug or therapy to a patient. One method for using the system as a biomarker is for the system to identify relevant published literature which indicates that a certain therapy is more likely or less likely to be effective for a patient with one or more data elements.

### III. Medical or Scientific Researcher as User

Within a hospital, there may be one or more researchers who are exploring different medical treatments or scientific questions. A hospital researcher may have access to the population of all the subject data in the hospital, without any identifying information for a particular patient or doctor. Through the system, the hospital researcher may use a variety of search and visualization tools in the system to examine different patient trends, subpopulations and/or correlations between different aspects of the subject (patient) data and changes to the subject data (outcomes). These trends, changes, correlations, visualizations, etc. may support or provide evidence against a researcher's hypothesis and possibly develop new avenues of scientific and medical discovery.

Another example is a medical or scientific researcher who is designing a clinical trial and needs to understand baseline subject data and subject predispositions for a patient population to design the protocol for a clinical trial. For example, a researcher might be testing a hypothesis that the introduction of a certain commensal microbe (virus, phage, etc.) and colonization at a certain location in the patient (e.g., small intestine) might improve the patient response to a certain diabetes drug. In order to understand how many patients to enroll and to set the trial endpoints, the researcher may look at subject data from a population to understand how common the commensal microbe is at that location within the subpopulation of diabetics and pre-diabetics, how frequently the introduction of the microbe via oral probiotics establishes a permanent colony of the microbe in the certain location within the population of diabetic patients, and the historical efficacy of the drug in treating those diabetics who have the microbe in sufficient quantity compared to those diabetics who do not have the microbe in sufficient quantity. Based on this information, the researcher could make more informed decisions on population sample size, enrollment criteria for patients and the target endpoints that would prove the scientific hypothesis.

Another example is for the researcher to use the subject prediction engine as a biomarker, precondition, complementary and/or companion diagnostic for use of a drug or therapy prior to enrollment in a trial or to modify the protocol during the trial. For example, if the subject prediction engine determines that a first subject is likely to respond well to a drug then this first patient may be included in the trial and if the subject prediction engine determines that a second subject is not likely to respond well to a drug then this second subject may be excluded from the trial.

Another example is for a researcher at a food and drink company to use the system for food and drink innovation. For example, the researcher may be considering a new food ingredient and uses the subject prediction engine to determine how the patients might respond to this new food by examining predispositions of the patients for changes to the patients' microbiome, likelihood of allergic reactions, etc.

Note that medical or scientific researchers might also be outside a hospital, such as at a medical device company, pharmaceutical company, governmental health agency, agricultural company, food and drink companies, patient advocacy group, etc.

### IV. Population Researcher

Outside the hospital, other researchers may be interested in using the system to explore different medical, environment, and/or societal questions. Example population researchers are epidemiologists, virologists, public health agencies, economic analysts, account manager, insurance company analysts, defense agencies, etc. A population researcher may have access to one or more populations or subpopulations of all the subject data in the hospital, without any identifying information for a particular patient or doctor. A population researcher may use this data in various ways, including but not limited to:
1. Examining changes to subject data in a certain geographical location or work environment
2. Comparing populations in different environments (e.g., air quality, water quality, weather patterns, etc.) to assess differences or anticipate problems
3. Epidemiological tracking of diseases or spread within a population
4. Assessment of economic correlations with population subject data, prediction of economic indicators or macroeconomic events
5. How frequently do certain microbes transmit between linked subjects (e.g., family members, co-workers, romantically involved partners, etc.).
6. Assessing whether a certain treatment is the most cost-effective way to deliver quality care to a patient or population
7. Assessing the appeal (e.g., reviews, engagement, likelihood of purchase, etc.) of certain products and services to populations or subpopulations of subjects with subject data that meets criteria set by the researcher
8. Assessing opportunities for preventative care. For example, assessing whether a certain population taking a probiotic, supplement or receiving certain coaching may prevent disease onset or otherwise avoid future costs. One method for using the system to assess these opportunities is for the researcher to set a cost reduction goal and a set of possible modifiable variables (e.g., diet, supplements, etc.) for a population of subjects and to receive guidance from the system on how best make changes to these modifiable variables to optimize cost.

### B. Individual Wellness

In this embodiment, it is described how an individual (or group of individuals) consumer could use the inventive concepts described herein to enhance and improve overall wellness.

In this embodiment, the following associations can be made:
1. Subject—The consumer
2. Subject-subject linkages—Other individuals who may share a microbial environment with the consumer, such as other individuals involved with the consumer in a shared living situation, romantic relationship, geography, workplace, environment, etc.
3. User—A consumer may have multiple users associated with them
   a. The consumer themselves
   b. The consumer's primary care physician
   c. The consumer's family member or spouse
4. Coaches—The consumer may have one or more of different coaches, including:
   a. Nutritionist
   b. Fitness coach
   c. Beauty coach
   d. Telehealth professional

In this embodiment the consumer may create their own account and user accounts for other users that the consumer designates or invite other users who have existing accounts to link to the consumer as a subject. The consumer may elect to share only some information with each user. The consumer may also invite or link to other consumers with a proposed subject-subject linkage. These linked consumers may or may not be asked to accept a linkage.

In this case, the subject/user context factors could be initialized through one or more of a variety of factors, including a questionnaire, connection with medical records, insurance data for the consumer, linking with social media accounts, mobile device data, online activity (e.g., purchasing history, etc.), wearable devices (e.g., fitness tracker, glucose monitor, etc.), etc. User context factors could be initialized for each user type in one or more of multiple ways, including user questionnaires, medical records, mobile device data, professional profile information, social media, online purchase history, etc. Following initialization, all of this data could be kept current by propagating changes to any of these data sources, such as be additional survey questions, social media updates, coach-user interactions, etc.

The consumer may adopt either a generic or average anatomical model to describe spatial location, medical imaging data of the consumer (e.g., MRI, ultrasound, CT, etc.), range camera, single or multiple optical camera data of the external body surface. If available, a digital anatomical model of the consumer is then created from this anatomical data by segmenting out each anatomical structure (including body surface) to create a digital volume and surface model for all anatomical structures for each patient (e.g., using multi-view camera algorithms, stitching algorithms, digital surface reconstruction techniques, etc.).

### I. Consumer Microbiome Sampling

Multiple different ways are contemplated in which the consumer may perform microbiome sampling. As with the healthcare provider scenario, the consumer could collect samples via:
1. Collection of one or more of the consumer's fecal samples, blood samples, urine samples, saliva samples, oral swabs, vaginal samples and/or respiratory samples for testing in a central hospital laboratory and/or external laboratory. Each of these samples being associated with the time of acquisition and location of sample (e.g., “GI tract”, “oral”, “bladder”, “lungs”, etc.).
2. Collection of one or more skin swabs, vaginal and/or oral swabs from multiple locations on the consumer's skin and oral cavity for testing in a central hospital laboratory and/or external laboratory. Each of these samples being associated with the time of acquisition. The location of these swabs could be selected on a display of a digital model for the consumer to match the location of the collected swab, selected from a drop down menu of possible locations, etc. The system records these sample locations and associates with the location the microbiome information obtained from the swab samples.

### i. Ingestible Capsule

Another scenario for the consumer to collect GI-tract located microbiome data is with one or more ingestible capsules. The consumer may or may not use a test capsule to determine whether a capsule can pass through the patient without difficulty. Calibration of one or more sensors may or may not be performed prior to ingestion. Prior to ingesting a capsule, the consumer may enter into their mobile device, receiver and/or computer a serial number, scan a barcode, scan a QR code, etc. on either the capsule or packaging to identify the capsule. A capsule may also have a transmitter (e.g., radio beacon) that transmits a unique code to a mobile device, receiver, etc. A capsule may be of any size that is effectively ingestible by the consumer (e.g., a 000 sized capsule). A capsule may or may not have one or more of the following elements:
1. One or more microbiome sampling mechanisms and/or sensors that collects and/or directly analyzes (either of transient material or collected material) fluid, microbiota, biofilms, tissue, gas, temperature, pH, etc. and is either later retrieved following excretion, tether and/or invasive means (e.g., surgery) and/or performs analysis in vivo and transmits that information to a receiver. There are multiple different methods for an ingestible capsule to collect and/or analyze samples, including but not limited to:
   a. Direct ingestion of fluid through an aperture or porous membrane. Such ingestion could be performed via multiple methods that are either entirely passive (due to capsule motion) or via an induced negative pressure, such as that created by osmosis or via an active device (e.g., a small motor), which may or may not include a discharge hole for excess fluid
   b. A self-polymerizing reaction mixture that entraps microbes and biomarkers
   c. Salt chamber capture (e.g., calcium chloride salt powder)
   d. Sponge
   e. Gas-permeable membrane (e.g., polydimethylsiloxane) with embedded nanomaterials that allow for the fast diffusion of dissolved gases (and potentially efficiently blocking liquid)
2. Microbiome sensors and analysis. One or more of many different types of sensor may be in the capsule, including but not limited to
   a. Gas sensor(s)
      i. Heating element modulation
      ii. Semiconductor with gas profile extraction algorithm
      iii. Electrochemical
      iv. Thermal conductivity
   b. Temperature sensor(s)
      i. Thermal conductivity
      ii. Quartz crystal, e.g., that vibrates at a frequency relative to the temperature, produces a magnetic flux and transmits a low-frequency signal
   c. Pressure sensor
   d. pH sensor
   e. Accelerometer
   f. Velocimeter
   g. Optical sensing device
      i. Camera
         1. Color sensing
      ii. Spectroscopy
      iii. Raman spectroscopy
      iv. Confocal microscopy
      v. Optical coherence tomography
      vi. Infrared
   h. Ultrasound
   i. Physisorption sensors
   j. Acoustic wave sensors (e.g., piezoelectric)
   k. Chromatography (e.g., pyroelectric)
   l. Fluorescence
   m. Electrochemical
   n. Sensors and/or analyses to measure microbe type and/or quantity, and/or other elements of the microbiome (e.g., proteins, neurotransmitters, cytokines, etc.). Such measurements may be performed in one or more of a variety of methods, including but not limited to:
      i. Biosensors and/or coupled with readout sensors (e.g., miniaturized luminescence, etc.).
      ii. One or more bacteria, probiotics or other biosensors may be designed and created via synthetic biology techniques to target response to one or more microbial elements (e.g., microbes, metabolites, viruses, phages, etc.).
      iii. Additional mechanisms include other biosensors such a protein biosensors that can be constructed from a system with two nearly isoenergetic states, the equilibrium between which is modulated by the analyte being sensed (e.g., LucCage and LucKey). Such biosensors may be designed to target response to one or more elements of the microbiome. These responses may be sensed, then recorded and/or transmitted to identify the targeted microbiome element.
      iv. One possible readout mechanism is for the bacteria or other biosensors to luminesce in response to the targeted microbiome element, which is detected by a photodetector which may or may not transmit the detection event to an outside receiver. In such a case, the biosensor probiotics could lie adjacent to readout electronics in individual wells separated from the outside environment by a semipermeable membrane that confines cells in the device and allows for diffusion of small molecules or other targeted elements of the microbiome.
      v. Enzyme catalyzation to create color or electrical output that may be coupled with readout sensors
3. One or more power sources, including but not limited to:
   a. Silver oxide coin batteries (e.g., 3V, 80 mA)
   b. Lithium ion batteries (with sufficient coating for safety)
   c. Remote (external) powering
      i. Inductive powering
      ii. Radiofrequency powering
      iii. Ultrasound
      iv. Optical (EM waves). Can be combined with multiple transmitters, beamforming, etc.
      v. Energy harvesting from body, for example
         1. GI internal acids
         2. Galvanic cell using Zn/Cu electrodes
         3. Piezoelectric nanogenerator generating electric power from a minuscule amount of deformation and vibration, such as GI forces, body heat, etc.
   d. Biofuel cells
4. One or more power switches, including but not limited to:
   a. Magnetic reed switch
   b. Remote (external) power on
   c. Exposure to energy harvesting conditions (e.g., gastric fluids)
5. One or more different materials could be used externally or internally with the capsule. For example, external materials being biocompatible and resistant to biofouling. Material examples, include, but are not limited to:
   a. Biocompatible cladding
      i. Rigid biocompatible polymers
      ii. Polyethylene
      iii. Nonionic hydrophilic materials
      iv. Amphiphilic materials
      v. Biocompatible photocurable polymers
   b. Zwitterionic polymer compositions
   c. Zinc oxides
   d. Silica
   e. Copper
   f. Iron
   g. Gelatin
   h. Cellulose and various
   i. Medical grade epoxy
   j. Silicone
   k. Hydrogels
   l. Poly(d, l-lactide-co-glycolide) and derivatives
6. One or more navigation methods through the GI tract, including but not limited to:
   a. Passive progression (e.g., motility)
   b. Active navigation
      i. Externally, e.g., with an on-board magnet that may be steered via an external magnetic source
      ii. Capsule based actuators
7. One or more time measurement devices to measure time of sensor readings, time since ingestion, time since activation and/or sample collection, including but not limited to microprocessor clock, crystal, etc.
8. One or more location measurement devices to measure location of sensor readings, location of capsule, and/or location of sample collection, including but not limited to:
   a. A sensor or transmitter that records location in absolute 3D space (world coordinates). For example, such a sensor may assess its position via triangulation with multiple known and calibrated beacons, transponders, etc. A sensor location may also be determined by external in vivo imaging of the consumer via x-ray, ultrasound, computed tomography (CT), magnetic resonance imaging (MRI), etc. In these cases, an opaque device may be attached to the sample collection mechanism to enhance the visibility of the imaging. The location determination via such imaging may be determined in many ways, such as by calibration of the imaging device (e.g., known instrument or patient positioning), manual reading of the images and recording the location, automated analysis of a digitized image with algorithms executed by an electronic processor, etc.
   b. A sensor or transmitter that generates location relative to subject coordinates. Relative position coordinates could be multidimensional.
      i. For an example of a three-dimensional coordinate, such a sensor or transmitter could assess position in space relative to one or more known beacons, transponders, to a calibration point placed on or inside the subject, etc. A reference point could also be obtained for the subject via one or more in the vivo imaging methods referred to above and using the imaging to map the location relative to that reference point.
      ii. For an example of two-dimensional coordinate, the sensor or transmitter location could be mapped to the surface of the subject's mucosal lining, GI tract, etc.
         1. This surface could have been previously mapped into a digital representation on an electronic storage device through multiple means such as time-of-flight imaging, structured light imaging, in vivo imaging such as x-ray, ultrasound, CT, MRI, sensor network (e.g., internal sensors), multiple point probes together with surface reconstruction algorithms. The location of the sample could be identified relative to this surface via either the same means used to create the surface (e.g., in vivo imaging of the sample on the surface) or mapped to the closest point on the surface if both the surface and the sample are located in the same coordinate system (world coordinates or relative coordinates).
      iii. For an example of a one-dimensional relative coordinate, a biological structure could be approximated by a one-dimensional space. For example, the GI tract could be approximated as a one-dimensional curved line (e.g., using the centroid of the cross-section of the GI tract to define a curved line) and determining the sensor or transmitter position along this one-dimensional space as a geodesic distance from a pre-defined origin location (e.g., mouth, anus, etc.). The sensor or transmitted position in the space could be determined via various means, such as:
         1. Using ex vivo imaging to measure the location of the sensor or transmitter and projecting that location onto the one-dimensional space.
         2. Measuring time elapsed since introduction of the sensor/transmitter and using accelerometer or velocimeter measurements (or assuming a predefined or user-input defined constant velocity) to determine travel within the approximately one-dimensional space.
   c. A sensor or transmitter that generates a categorical location relative to the subject. In other cases, the anatomical location may be determined automatically by measuring biological characteristics surrounding the sample acquisition, such as pH, local gas composition (oxygen, oxygen-equivalent concentration profile, hydrogen, nitrogen, carbon dioxide, etc.), temperature, etc., and matching those measured biological characteristics with known biological characteristics of different locations. For example, a sample acquired in the GI tract that measured a surrounding pH between 1.5-3.5 can be determined to have been taken in the stomach. Categorical locations may be defined narrowly (e.g., proximal duodenum) or broadly (e.g., GI tract) and may be defined in an anatomical or functional taxonomy describing relationships between different categorical locations (e.g., proximal duodenum is part of duodenum which is part of the small intestine which is part of the GI tract, etc.). Examples of categorical locations may include any anatomical locations, sublocations, etc.
   d. A location-activated switch that triggers sample collection at a particular location. A location-activated switch using any of the same mechanisms (such as those below) could also be used to turn off sample collection and therefore limit sample collection to a target location or set of locations. By specifying a location-activated switch, the system knows that the sample was taken from the location specified by the switch. In this case, the term switch refers to any change of device state that initiates sample collection. There are many such types of location-activated sample collection switches, which may include but are not limited to the following mechanisms:
      i. Activation after a certain time (e.g., by electronically or magnetically opening/closing gates to allow fluidic capture), where the location could be determined by assessing the expected location of the device after a certain time (possibly also using accelerometer or velocimeter readings).
      ii. A GI-ingestible device could be coated such that the coating dissolves at a target location (due to pH, enteric coating, etc). One such example is Cellulose Acetate Phthalate.
      iii. The switch of a sampling device could be made to trigger electronically by any biological profile associated with a certain location, such as pH, local gas composition (oxygen, hydrogen, nitrogen, carbon dioxide, etc.), local chemical concentration, local microbiome sampling composition, local sweat, local temperature, etc. A switch could also be made to trigger mechanically via different mechanisms in response to a biological profile, such as a hydrogel that responds by swelling to initiate or terminate sample collection.
      iv. Magnetically opened and closed sampling (triggered) for sampling at different locations
      v. Electrically powered sample collection that is triggered by a magnetic reed switch or exposure to gastric acids to generate power
      vi. A machine learning or statistical method could be trained by collecting a series of known locations and biological profiles to identify a location by a biological profile and to trigger the sample collection when the biological profile was determined to match the biological profile of a known location
   e. Active movement of the sampling device to a target location via a control mechanism. This control mechanism could be performed via a range of methods, including manual manipulation, actuators controlled via wired or wireless controllers, moving the sampling device with one or more external magnets, etc. This control mechanism may or may not include feedback for the sampling operator.
9. One or more different communication mechanisms to communicate externally, for example to transmit and/or receive sensor data, time/location information, body exit signal, sample information, etc. External communication may also enable a kill signal to be sent to the capsule to disable a device (e.g., a malfunctioning device). Note that any communication signals may or may not have different features, such as security (e.g., signal encryption), compression, etc. Communication with the capsule may be performed from one or more of multiple devices such as a mobile device (e.g., smartphone), wearable transceiver, etc. Communication devices may or may not conform to the Medical Implant Communication Service (MICS), which is a 402-405 MHz band that is a licensed band for diagnostic and therapeutic medical implants and body-worn medical devices. These communication mechanisms may take many different forms, including but not limited to:
   a. Radiofrequency antenna (e.g., 433 MHz)
   b. Outer wall loop antenna connected to a high-speed, high-efficiency transceiver system
   c. Miniaturized flexible antennas
   d. Using the subject body as a transmission medium. For example, utilizing gold electrodes for transmitting data and an array of electrodes attached to the human skin to receive data, using the human body as an electronic conductor
   e. Bluetooth
   f. Quartz crystal
   g. MICS transceiver with coverage of ±160 ppm carrier frequency offset and a 4.8-VSWR antenna impedance
10. One or more microcontrollers, processors and/or electronic storage devices (e.g., a circuit board). Such on-board processing may be used for multiple purposes, including but not limited to:
   a. In vivo analysis of samples and/or sensors
   b. Interfacing with other components (e.g., sensors, actuators, communication devices, storage devices, etc.)
   c. Error correction of sample and/or sensor data (e.g., self-supervised learning)
   d. Location detection
11. One or more elements to aid ex vivo retrieval (finding and retrieval), including but not limited to:
   a. Dyes (e.g., to color stool for the capsule)
   b. Magnets (e.g., to enable an external magnet to find and retrieve)

For clarity, note that the consumer may or may not ingest multiple capsules that include one or more different or the same elements described above to achieve sufficient sampling, sensing, monitoring, etc. Multiple capsules may be ingested at multiple different time intervals, (e.g., at the same time, evenly spaced, on a predefined schedule, etc.). For example, the consumer may ingest a first capsule to measure body temperature at different locations, a second capsule with a biosensor designed to detect internal bleeding (heme) at different locations, a third capsule with a biosensor designed to detect and quantify nutritional content (e.g., carbohydrates) at different locations, a fourth capsule with a biosensor designed to detect and quantify a first microbe (e.g., Escherichia coli), a fifth capsule with a biosensor designed to detect and quantify a second microbe (e.g., Lactobacillus gasseri), etc. For clarity, note that each capsule sample may be analyzed in vivo (e.g., via on-board sensors, analysis, transmission, etc.), ex vivo (e.g., via retrieval and lab analysis) and/or a combination thereof. Each capsule may be analyzed differently (e.g., one capsule analyzes and transmits signals in vivo, a second capsule is retrieved for ex vivo analysis, a third capsule is retrieved for data transfer of on-board analysis, etc.).

One or more capsules could be manufactured in many different ways. Some examples include, but are not limited to a 3D printer, stereolithography (e.g., biocompatible methacrylate photocurable polymer), high temperature resin (possibly including photo-curing), etc. A hydrophilic surface modification may be performed on the surface of 3D-printed housing to ensure and facilitate sampling fluid to enter the capsule's aperture (e.g., via surface activation with an air plasma treatment followed by poly-ethylene glycol treatment).

### II. Consumer Use of the System

After supplying one or more elements of subject/user context data and performing at least one microbiome sampling, multiple ways are subsequently described in which the consumer may use the system in this embodiment. The user may access their system account via one or more device, including but not limited to a mobile device (e.g., smartphone, tablet, etc.), computer, laptop, network connection, wifi connection, satellite link, etc.

### i. Subject/User Predispositions

The user may look through all of their subject/user predispositions to identify health risks, traits, etc. to generally assess their health, wellness, receive recommendations, track changes in predispositions across multiple time points (e.g., microbiome samples, changes in context factor, etc.), purchase products and to compare themselves to other populations (e.g., a total population, local geography, people with a certain medical condition, etc.). Some possibilities include, but are not limited to:
1. If the user identifies that they might be at high risk for a health condition, the user may send a message to their doctor and/or invite their doctor as a user of the system with access to one or more elements of subject/user data (e.g., perhaps selected by the user).
2. The user may identify that some predispositions are concerning (e.g., elevated risk of diabetes) and set an alert that if the subject's predisposition crosses a preset threshold, or approaches the preset threshold above a predetermined rate, they may receive a push notification and/or an email.
3. The user may access educational materials through the system to learn more and better understand many different topics including, but not limited to, medical conditions, subject/user predispositions, nutrition, pregnancy, fitness, wellness, etc. Additionally, the subject/user may explore links between their microbiome and/or other subject/user data. The user prediction engine may also suggest or recommend personalized or customized educational material (or configurations, curricula, etc. of existing educational material) for the user. The user may also search, filter and request educational material.
4. The user may create hypothesized subject data by adjusting some of their data elements (e.g., weight, sleep habits, diet, fitness, vitamins, pregnancy status, getting a new pet) in order to determine the impact of these changes on subject predispositions produced by the subject prediction engine. Perhaps the user identifies that small changes to the user's sleep habits, fitness and vitamins could have a significant positive impact on the user's predispositions. As a result, the user is recommended certain vitamins and fitness programs in the system marketplace (e.g., online store) which can most benefit the user. The user may read/write reviews, monitor, learn more, purchase, etc. these products through the platform.
5. The user may learn through the system that there is a study or clinical trial that the user is eligible for. As a result, the user may use the system to indicate interest, enroll online, contact their doctor, etc. to possibly get involved in the study or trial.
6. The user may learn through the system that there is a new product or service offer that the user is eligible for. As a result, the user may use the system to indicate interest, enroll online, purchase, contact the vendor, etc. to possibly engage with the product or service.
7. The user may also engage with the system to initiate and/or evaluate subject prediction engine determinations of future states, e.g., likely evolution of the user's microbiome, menstrual cycle, etc.
8. Engage with one or more features detailed in the user guidance section
9. Engage with social media accounts that are linked to the user's system account. For example:
   a. The user choosing to sharing some of their user/subject data and/or predispositions on the social media platform
   b. Receiving suggestions for new linkages (e.g., based on a user and their spouse both being on a social network)
   c. Receive dating suggestions on a linked dating application. For example, similarities in the two individuals' microbiome (and/or context data) may suggest a higher predisposition for a successful match
10. The user may use the system to assess how well certain beauty or cosmetic products work for them. For example, to assess subject predispositions for certain shampoos to affect the quality of their hair in different ways, likelihood that certain makeup or skin creams will cause dry skin, how long lasting certain lipsticks or nail polish are likely to last on the subject's skin. The user may also receive recommendations for beauty and cosmetic products and services, as well as have the ability to purchase products and services in a marketplace through the system.
11. The user may assess the change, prevalence, incidence and/or spread of a microbe, pathogen, condition or disease with the system. Some examples include, but are not limited to, the user might look at a display visualizing the spread of an infectious disease (e.g., influenza) within different geographies, the sharing of a microbe between linked subjects, prevalence of a medical condition for all subjects sharing the same water supply, co-incidence of diabetes and heart disease in an older population, prevalence of a skin dryness resulting from use of a certain cosmetic, etc. These displays may be static or show changes over time. For clarity, in some cases a second user may have to explicitly share data for the other user's data to be included or accessible in any way with the user.

### ii. Exemplary User Interface

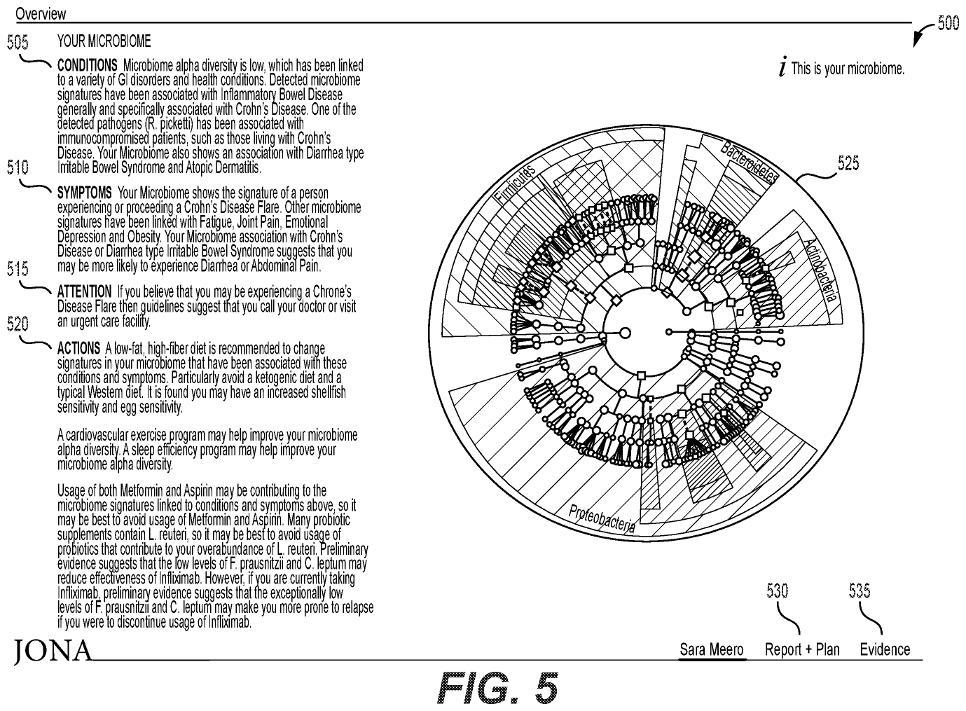

Referring now to FIGS. 5-8, an exemplary user interface is provided according to one or more embodiments. The exemplary user interface is populated with sample data for an imaginary individual—“Sara”. In these non-limiting examples, Sara is the subject and may also be a user.

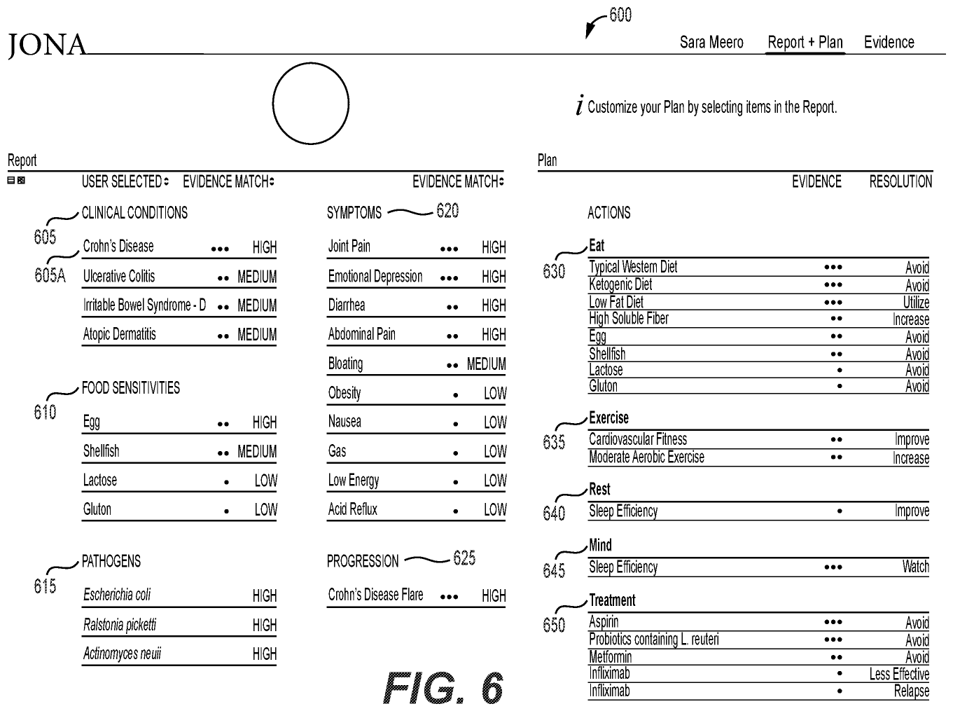

Referring now to FIG. 5, a summary and visualization page 500 is illustrated. This page 500 may provide various types of information to the user, such as: the conditions 505 associated with the subject's microbiome, the projected symptoms 510 of an individual that may share the same microbiome conditions as the subject, a notification alert 515, and/or one or more recommendations for actions 520 the subject could take to alleviate one or more of the conditions and/or symptoms. For example, this summary overview may be generated automatically via an NLP system and/or via a template based on the detailed recommendations. Additionally to the foregoing, the page 500 may provide the user with an exemplary visualization 525 of the subject's microbiome. Please note that the visualization 525 is a non-limiting example of how a subject's microbiome may be presented and other visualizations, of varying styles and/or containing more or less information, may be provided. A user may navigate away from the screen represented by FIG. 5 via selection of one or more icons located on the bottom of the screen, .e.g., a Report+Plan icon 530 or an Evidence icon 535. For example, the user may navigate to an overview summary screen 600, as represented in FIG. 6, via selection of a “Report+Plan” icon 530.

In an embodiment, the overview summary screen 600 may contain a representative summary of a variety of characteristics associated with the subject. This information may be organized into sections (e.g., Clinical Conditions 605, Food Sensitivities 610, Pathogens 615, Symptoms 620, Progression 625, etc.) that each contain various elements associated with that section. Additionally, each element may contain a corresponding indication of how strongly the subject's microbiome matches what has been described in the literature for the condition and the level of evidence describing that match under the relevant section. For example, the Clinical Conditions section 505 may contain four listed conditions for which there is evidence in the literature describing a microbiome composition associated with a clinical condition that matches the subject's microbiome composition—Crohn's Disease, Ulcerative Colitis, Irritable Bowel Syndrome— D, and Atopic Dermatitis. In this example, the system states that the subject's microbiome closely matches the microbiome of people with Crohn's Disease with significant evidence with the other listed conditions being designated a medium level of evidence. In an embodiment, the overview summary screen 600 may also provide a summarized listing of characteristics of an action plan that a subject can implement to adjust their microbiome in such a way that it would lower the associations with clinical conditions. The information in the action plan may be organized into sections (e.g., Eat 630, Exercise 635, Rest 640, Mind 645, Treatment 650, etc.) that each contain various elements associated with that section. Additionally, each element main contain a corresponding indication of a recommended action the subject can take with respect to that element (e.g., avoid consumption of certain foods, increase exercise of a particular type, avoid certain medications, etc.). Additionally or alternatively to the foregoing, the overview summary screen 600 may also provide an indication of associations of the subject's current microbiome with symptoms, conditions and progressions.

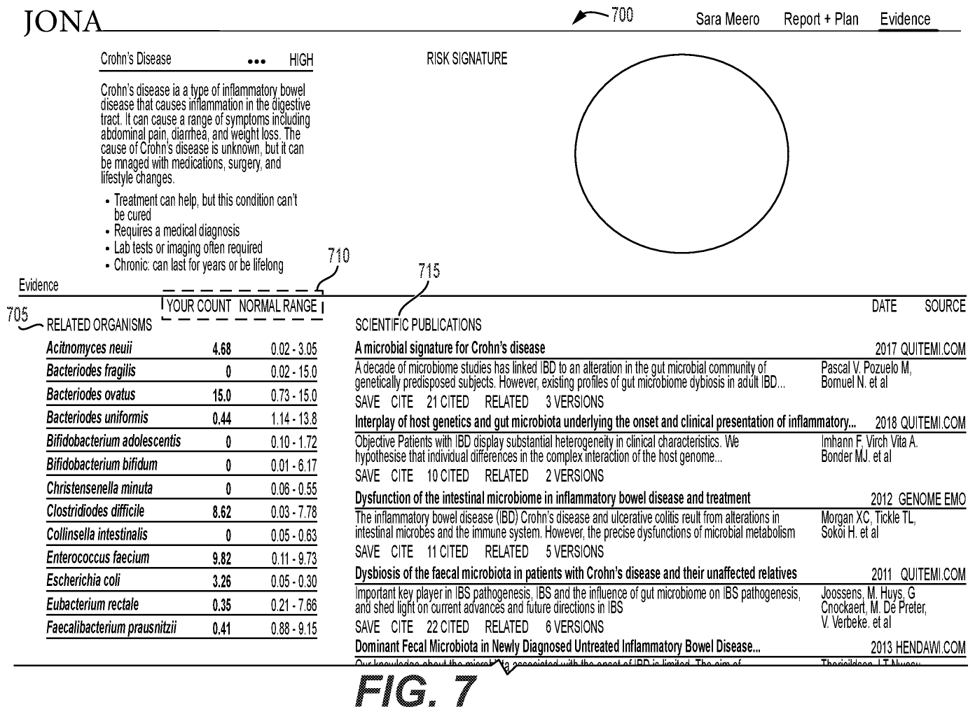

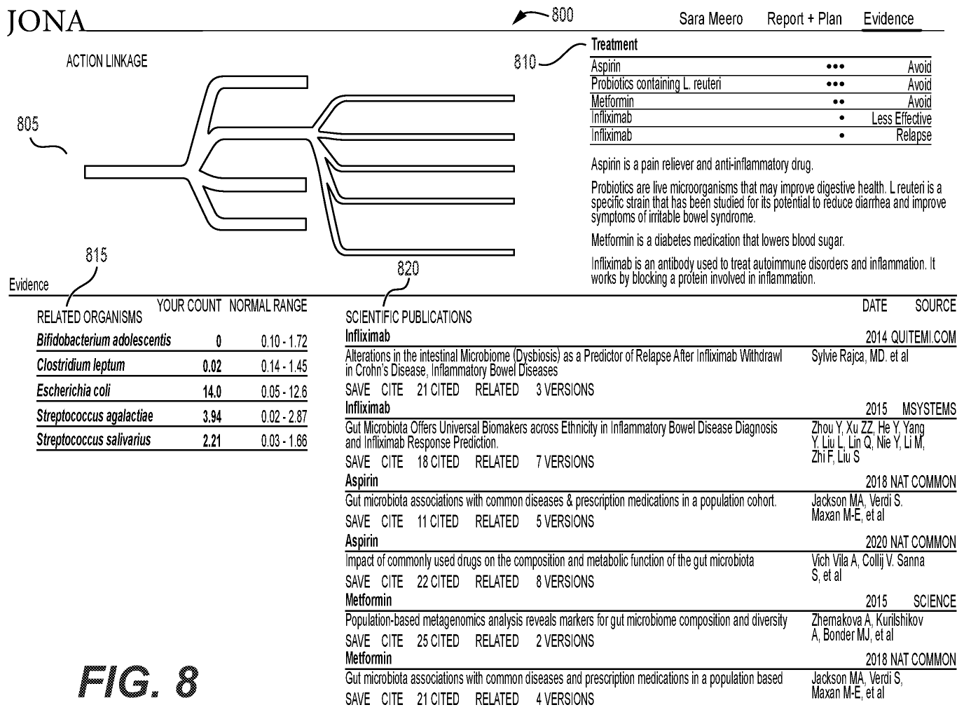

In an embodiment, a user may be apprised of additional information associated with each section or element by interacting with a desired icon. For example, a user interested in obtaining more detail about the subject's microbiome association with Crohn's Disease may select the Crohn's Disease element 605A under the Clinical Conditions section 605. Upon selection, a user may be redirected to a dedicated Crohn's Disease page 700, such as the one illustrated in FIG. 7. In an embodiment, the Crohn's Disease page 700 may contain a variety of different information about Crohn's Disease such as a summarized definition of the disease 700, organisms and levels that have been associated with the disease that were measured to be in the subject 705, prevalence of the related organisms in the subject's microbiome with respect to a normal range 710, scientific publications linking the related organisms to the clinical condition 715, and the like. As another example, a user interested in obtaining additional information about treatment options may select the Treatment section header 650. Upon selection, a user may be redirected to a dedicated treatment page 800, such as the one illustrated in FIG. 8. In an embodiment, the dedicated treatment page 800 may contain a variety of different types of treatment-related information such as: a visualization of an action linkage 805; medication listings and summaries associated therewith 810, an indication of organisms that have been shown in the literature to interact with the treatment (i.e., those organisms that may be directly affected by certain medications) 815, publications linking the medications with the related organisms 820, and the like.

### iii. Goals and Coaching

The user may decide that the user wants to improve their sleep quality and reduce back pain. The user may also want to maximize their chance of pregnancy (fertility). The user may use the system for this purpose by setting goals and establishing which factors the user is able to modify. The user may either select or be matched with one or more coaches to support the user in their goals. In this example, the user might be matched with three separate coaches to help the user achieve their goals: A sleep coach, a physical therapy coach and a pregnancy and fertility coach.

The user may engage with these coaches via one or more mechanisms including, but not limited to, regular (e.g., biweekly) video/phone discussions, on-demand, as the user needs through messaging, etc. The user may also choose to interact with (read/write) message boards, possibly including one or more other users/coaches. The user may also engage with chatbots through the system to help answer questions and learn more.

Based on coach data, user data, subject data, user-coach interaction data and historical data in the system, each coach may receive one or more guidances to help the coach guide the user interactions between each coach and the user to help the user achieve their goals of better sleep, increased chance of pregnancy and reduced back pain. As the user achieves one or more of these goals, the user may elect to set new goals and/or shift their interaction with these coaches into maintenance and monitoring.

### III. Virtual Trials or Studies

Clinical trials and studies are expensive, time consuming and risky. However, a clinical trial or study is often the only way for a clinical researcher, population researcher or industrial researcher at a drug company, medical device company, food and beverage company, cosmetic company, beauty company, animal supply company, agricultural company, wellness company, etc. to assess the effectiveness and possible safety or side effects of a new treatment, device or product. In this embodiment, it is described how the inventive concepts could be used to perform a virtual trial or study that could substantially reduce costs, ensure safety and improve speed of a trial or study.

Consider a company who wants to test or evaluate a new product. In this example, a new drug to reduce Mild Cognitive Impairment (MCI) is contemplated, which is often a precursor to Alzheimer's disease. The company may target the drug at non-diabetic people over 50. The researcher could use the inventive concepts described herein to perform a virtual clinical trial of this drug by following these steps:
1. Using the system to create a new population of virtual subjects, where the number of virtual subjects is set to provide sufficient statistical power for the study
2. Set subject context factors for the virtual subjects to set their age to a distribution of ages over 50 (e.g., uniformly distributed between 50 and 90, etc.)
3. Set subject context factors for the virtual subjects that each virtual subject's diabetes status is set to negative
4. Optionally establish a distribution of one or more additional elements of subject data for the virtual subjects, e.g., by sampling different genders, ethnicities, locations, etc. such that the researcher believes that the distribution represents a representative sample of the target population
5. Optionally the researcher could pre-specify virtual clinical trial endpoints prior to the virtual clinical trial
6. For any elements of the subject data not defined by the researcher, the system could fill in one or more data elements for the virtual subjects by sampling from the distribution of subjects in the database and/or using published sources. For example, if dietary or microbiome information is not established for these virtual subjects, then for each virtual subject (for example, a virtual 55 year old woman, with high education, low socioeconomic status, living in Houston, etc.) the system could fill in the missing dietary or microbiome data in one of multiple different ways, including but not limited to, by sampling likelihoods (e.g., marginal or conditional probabilities) from the system subject database, referencing publication sources, the subject prediction engine to produce subject predispositions for these subject data elements, etc.
7. The researcher could then divide the population of virtual subjects into a control and test group
8. The researcher could then set the subject data in the virtual test group to include use of the new drug and/or drug's underlying compounds, mechanism of action, etc. while the subject data for the virtual control group does not include this new drug.
9. The researcher could then apply the subject prediction engine to both the virtual subjects in the control group and the virtual subjects in the test group. The subject predispositions for both groups could be compared using standard biostatistical methods to assess whether the drug was effective in reducing MCI for the virtual test subjects compared to the virtual control subjects (e.g., by comparing the subject predispositions for MCI in the virtual test group to the virtual control group) and/or whether the drug had any toxic, side effects or off-target effects by comparing the subject predispositions of the virtual test group with the virtual control group.
10. If endpoints for the virtual clinical trial were specified, evaluate these endpoints with the virtual clinical trial data

For clarity, this virtual clinical trial could first be run on virtual mice subjects prior to a real mouse trial and then run on virtual human subjects prior to a real human trial.

The same methodology for conducting virtual trials or studies could be used to evaluate a variety of new products and services (clinical, consumer, animal, plant, etc.) including but not limited to new food products (e.g., comparing subject predisposition for food preference in virtual control subjects versus virtual test subjects), new medical devices (e.g., comparing subject predisposition for patient outcome in virtual control subjects versus virtual test subjects), new animal food products (e.g., comparing subject predisposition for milk production quality and quantity in virtual control subjects versus virtual test subjects), new plant fertilizer products (e.g., comparing subject predisposition for crop yield quality and quantity in virtual control subjects versus virtual test subjects), new beauty or cosmetic products (e.g., comparing subject predisposition for consumer dry skin in virtual control subjects versus virtual test subjects of a new moisturizer), etc.

### IV. Forensics

The inventive concepts described herein may also be utilized in the context of forensics to help an investigator or relative better understand a deceased subject. In this embodiment, the following associations may be made:
1. Subject—The deceased (human, animal or plant)
2. Subject-subject linkages—Other individuals who may have shared a microbial environment with the deceased, such as other individuals involved with the deceased in a shared location of death, shared living situation, romantic relationship, geography, workplace, environment, etc.
3. User—A deceased may have multiple users associated with them, including but not limited to:
   a. An investigator (e.g., detective, autopsy physician, etc.)
   b. A relative or family member of the deceased
   c. Pet, animal or plant owner (e.g., if the deceased is an animal or plant)
4. Coaches—A user may have one or more of different coaches, including but not limited to:
   a. Grief counselor
   b. Law enforcement
   c. Medical specialist
   d. Forensics expert
   e. Agricultural expert

In this embodiment, subject context factor data may be supplied in one or more of multiple ways, including but not limited to, being entered by one or more users, having been entered previously be the deceased (e.g., if the deceased had an account with the system while still alive), hospital/medical/morgue records (e.g., EMR, PACS, LIS, etc.), farm or agricultural records, law enforcement records/database, etc. Microbiome information about the subject may or may not be obtained in one or more ways, including but not limited to taking biological samples (e.g., fluids, tissue, hair, biofilms, etc.) with one or more of the methods described previously (e.g., swabs, biopsy, fluid collection, surgical removal, autopsy, etc.).

The user may use the subject prediction engine to assess a variety of factors, including subject predispositions about subject mortality (e.g., cause of death, time of death, etc.) as well as assess other subject predispositions that may provide important information (e.g., likely health risk factors that were being untreated, etc.). An investigator may also decide to adjust some hypothesized subject data to account for unknown information and assess different scenarios with the subject prediction engine. If an investigator needed assistance (e.g., advice, further evidence gathering, more information, etc.), the investigator may benefit from accessing a coach such as a law enforcement professional, medical specialist, forensics expert, etc.

An aggrieved family member user may want to use the system on their own in one or more of many different ways, including but not limited to investigating the deceased on their own by learning more about the deceased (e.g., via subject data or determined subject predispositions), adjusting hypothesized subject data to determine the effect of subject predispositions, accessing and interacting with message boards, accessing (possibly personalized and customized) educational materials, accessing related products and services on a marketplace, talking to a grief counselor (coach), etc.

### V. Animal and Plant Health

Understanding of plant and animal health and wellness is important for a wide variety of uses, including agriculture (livestock), farming (crops), fishing, veterinary, medical, etc. The inventive concepts described herein may be used to improve animal and plant health, improve agricultural output and better manage environmental resources. In this embodiment, the following associations may be made:
1. Subject—One or more animals and/or plants
2. Subject-subject linkages—Other animals and/or plants that may share a microbial environment with a subject, such as other animals and plants who share a living environment, food/water/soil source, animals or plants who might be visited by the same pests, mating partners, etc.
3. User—One or more animals and/or plants may have one or more users associated with them, including but not limited to
   a. A farm owner or employee
   b. A pet owner
   c. A fishing professional
   d. A veterinarian
4. Coaches— A user may have one or more of different coaches, including but not limited to
   a. Medical specialist
   b. Agricultural specialist
   c. Fishing specialist

In this embodiment, subject context factor data may be supplied in one or more of multiple ways, including but not limited to, being entered by one or more users, veterinary records, agricultural records (database), fishery records (database), atmospheric databases, water sensors/samples, air sensors/samples, soil sensors/samples, consumer surveys/databases (e.g., if a particular fruit (e.g., plant and/or animal product) was deemed better quality by consumers, sold for a higher price, etc.), etc. Microbiome information about one or more subjects may or may not be obtained in one or more ways, including but not limited to taking biological samples (e.g., fluids, tissue, hair/fur, biofilms, roots, stems, leaves, etc.) with one or more of the methods described previously (e.g., swabs, biopsy, fluid collection, resection, autopsy, etc.). In some situations, there may be more subjects than can be easily measured (e.g., a farm with millions of wheat plants). In these cases, the user may or may not elect to sample a subset of subjects in the population and create one or more virtual subjects (e.g., in a manner similar to the virtual clinical trial embodiment) to represent one or more of the unsampled population.

User context factors could be initialized for each user type in one or more of multiple ways, including user questionnaires, professional accounts, professional profile information, social media, online purchase history, etc. Following initialization, all of this data could be kept current by propagating changes to the subject data for a subject, additional survey questions, social media updates, coach-user interactions, etc.

After supplying one or more elements of subject/user data, multiple ways are subsequently described in which a user may use the system in this embodiment. A user may access their system account via one or more devices, including but not limited to a mobile device (e.g., smartphone, tablet, etc.), computer, laptop, network connection, wireless (Wi-Fi) connection, satellite link, etc.

The user may look through all of the subject predispositions to identify health risks, traits, etc. to generally assess animal or plant health, wellness, receive recommendations, track changes in predispositions across multiple time points (e.g., microbiome samples, changes in context factor, etc.), purchase products and to compare the animal and/or plant subjects to other populations (e.g., a total population, local geography, similar farms, similar fishing environments, etc.).

Some specific examples of ways in which the user might use the system include, but are not limited to:
1. If the user identifies that one or more animals or plants might be at high risk for a health condition, the user may send a message to their veterinarian, agricultural specialist, fishery specialist, etc. and/or invite one or more of these individuals as a user of the system with access to one or more elements of subject/user data (e.g., perhaps selected by the user).
2. The user may identify that some predispositions are concerning (e.g., elevated risk of disease, elevated risk of reduced milk quality, etc.) and set an alert that if one or more subjects' predisposition(s) cross a preset threshold, the user will receive a push notification and/or an email.
3. The user may access educational materials through the system to learn more and better understand many different topics including, but not limited to, medical conditions, crop rotation, livestock management, subject predispositions, nutrition, pregnancy, fitness, wellness, best practices, governmental warnings/alerts, etc. The user prediction engine may also suggest or recommend personalized or customized educational material (or configurations, curricula, etc., of existing educational material) for the user. The user may also search, filter and request educational material.
4. The user may create hypothesized subject data for one or more subjects by adjusting one or more of the subjects' data elements (e.g., soil, milking frequency, crop rotation, weight, sleep habits, feed, fertilizers, exercise, vitamins, antibiotic usage, pregnancy status, introduction or removal of plants or animals) in order to determine the impact of these changes on subject predispositions produced by the subject prediction engine. Perhaps the user identifies that small changes to the animals' feed or a crop's water schedule could have a significant positive impact on the subject's predispositions. As a result, the user is recommended certain feed and irrigation programs in the system marketplace (e.g., online store) which can most benefit the user. The user may read/write reviews, monitor, learn more, purchase, etc. these products through the platform.
5. The user may learn through the system that there is a study or clinical trial that one or more subjects associated with the user is eligible for. As a result, the user may use the system to indicate interest, enroll online, request more information, etc. to possibly get involved in the study or trial.
6. The user may learn through the system that there is a new product or service offer that the user is eligible for. As a result, the user may use the system to indicate interest, enroll online, purchase, contact the vendor, etc. to possibly engage with the product or service.
7. The user may also engage with the system to initiate and/or evaluate subject prediction engine determinations of future states, e.g., likely evolution of the one or more subjects' microbiome, egg production yield over time, etc.
8. Engage with one or more features detailed in the user guidance section
9. Engage with social media accounts that are linked to the user's system account. For example:
   a. The user choosing to share some of their subject data and/or predispositions on the social media platform
   b. Receiving offers for related products and services
10. The user may assess the change, prevalence, incidence and/or spread of a microbe, pathogen, condition or disease with the system. Some examples include, but are not limited to, the user might look at a display visualizing the spread of an infectious disease (e.g., foot-and-mouth disease) within different geographies, the sharing of a microbe between linked subjects, prevalence of a medical condition for all subjects sharing the same soil, feed and/or water supply, co-incidence weight loss in an older population, prevalence of lower wool quality resulting from certain environmental factors, etc. These displays may be static or show changes over time.

If a user needed assistance (e.g., advice, more information, etc.), a user may benefit from accessing a coach such as a veterinarian, agricultural specialist, fishery specialist, etc. which the user may do through the coaching functionality. The user may also set one or more goals (e.g., improved dairy production, restoring a fishery population, improved crop yields, cost reduction, etc.). To help the user achieve this one or more goals, the system may make one or more recommendations using the user prediction engine and/or one or more coaches may be matched to assist the user to achieve these goals. As above, the coach may also receive recommendations based on user-coach interactions to help the coach be more effective in helping the user achieve their one or more goals.

The systems, apparatuses, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these the apparatuses, devices, systems, or methods unless specifically designated as mandatory. For ease of reading and clarity, certain components, modules, or methods may be described solely in connection with a specific figure. In this disclosure, any identification of specific techniques, arrangements, etc. are either related to a specific example presented or are merely a general description of such a technique, arrangement, etc. Identifications of specific details or examples are not intended to be, and should not be, construed as mandatory or limiting unless specifically designated as such. Any failure to specifically describe a combination or sub-combination of components should not be understood as an indication that any combination or sub-combination is not possible. It will be appreciated that modifications to disclosed and described examples, arrangements, configurations, components, elements, apparatuses, devices, systems, methods, etc. can be made and may be desired for a specific application. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Throughout this disclosure, references to components or modules generally refer to items that logically can be grouped together to perform a function or group of related functions. Like reference numerals are generally intended to refer to the same or similar components. Components and modules can be implemented in software, hardware, or a combination of software and hardware. The term “software” is used expansively to include not only executable code, for example machine-executable or machine-interpretable instructions, but also data structures, data stores and computing instructions stored in any suitable electronic format, including firmware, and embedded software. The terms “information” and “data” are used expansively and includes a wide variety of electronic information, including executable code; content such as text, video data, and audio data, among others; and various codes or flags. The terms “information,” “data,” and “content” are sometimes used interchangeably when permitted by context.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method of leveraging subject microbiome data, the computer-implemented method comprising operations including:
detecting, on a graphical user interface of an application platform of a user computing device, a selection of a subject by a user;
accessing, based on the detecting, data associated with the subject, wherein the data comprises the subject microbiome data;
generating, by a processor, an overview report comprising a first set of subject predispositions; and
displaying, on the application platform, the generated overview report.

2. The computer-implemented method of claim 1, wherein the generated overview report comprises: a list of expected conditions associated with the subject microbiome data, an expected symptom set associated with the subject microbiome data, and one or more recommended actions to improve a condition of a subject microbiome based on the subject microbiome data.

3. The computer-implemented method of claim 1, further comprising:
receiving, at the application platform, user modification input that adjusts one or more elements of the first set of subject predispositions; and
identifying, using the processor, a predictive change to the subject microbiome data based on the user modification input.

4. The computer-implemented method of claim 3, further comprising:
generating, using the processor, a second set of subject predispositions based on the predictive change; and
displaying, on the application platform and subsequent to the generating, the second set of subject predispositions.

5. The computer-implemented method of claim 4, wherein the displaying the second set of subject predispositions comprises visually distinguishing, on the graphical user interface, differences between the first set of subject predispositions and the second set of subject predispositions that are greater than a predetermined threshold.

6. The computer-implemented method of claim 1, further comprising:
receiving, at the application platform, an indication of a target goal for the subject;
retrieving, using the processor, microbiome data of a reference subject having achieved the target goal;
comparing, using the processor, the subject microbiome data to the microbiome data of the reference subject;
identifying a change to one or more elements of the first set of subject predispositions needed to adjust characteristics of the subject microbiome data to the microbiome data of the reference subject;
generating a second set of subject predispositions that incorporates the change; and
displaying, on the application platform, a plan that includes the second set of subject predispositions.

7. The computer-implemented method of claim 6, wherein the plan comprises a list of recommended actions the subject should take for each of the second set of subject predispositions to achieve the target goal.

8. The computer-implemented method of claim 6, further comprising:
determining, based on the target goal and/or the data associated with the subject, a coaching individual; and
automatically matching, based on the determined coaching individual, the coaching individual with the user.

9. The computer-implemented method of claim 8, further comprising:
identifying interaction data between the coaching individual and the subject;
analyzing, using the processor, the interaction data with respect to a progress rate of the user toward the target goal;
determining, using the processor and based on the analyzed interaction data, an aspect of the interaction data that accelerates or impedes the progress rate of the subject toward the target goal; and
transmitting, by the user computing device, instructions to another computing device associated with the coaching individual to display the aspect.

10. The computer-implemented method of claim 1, wherein the subject microbiome data is derived from a microbiome sample from the subject and/or a second microbiome sample from a second subject that is linked to the subject.

11. A user computing device, comprising:
one or more computer processors; and
a non-transitory computer-readable storage medium storing instructions executable by the one or more computer processors, the instructions when executed by the one or more computer processors causing the one or more computer processors to perform operations including:
detecting, on a graphical user interface of an application platform associated with the user computing device, a selection of a subject by a user;
accessing, based on the detecting, data associated with the subject, wherein the data comprises the subject microbiome data;
generating, by a processor, an overview report comprising a first set of subject predispositions; and
displaying, on the application platform, the generated overview report.

12. The user computing device of claim 11, wherein the generated overview report comprises: a list of expected conditions associated with the subject microbiome data, an expected symptom set associated with the subject microbiome data, and one or more recommended actions to improve a condition of a subject microbiome based on the subject microbiome data.

13. The user computing device of claim 11, further comprising:
receiving, at the application platform, user modification input that adjusts one or more elements of the first set of subject predispositions; and
identifying, using the processor, a predictive change to the subject microbiome data based on the user modification input.

14. The user computing device of claim 13, further comprising:
generating, using the processor, a second set of subject predispositions based on the predictive change; and
displaying, on the application platform and subsequent to the generating, the second set of subject predispositions.

15. The user computing device of claim 14, wherein the displaying the second set of subject predispositions comprises visually distinguishing, on the graphical user interface, differences between the first set of subject predispositions and the second set of subject predispositions that are greater than a predetermined threshold.

16. The user computing device of claim 11, further comprising:
receiving, at the application platform, an indication of a target goal for the subject;
retrieving, using the processor, microbiome data of a reference subject having achieved the target goal;
comparing, using the processor, the subject microbiome data to the microbiome data of the reference subject;
identifying a change to one or more elements of the first set of subject predispositions needed to adjust characteristics of the subject microbiome data to the microbiome data of the reference subject;
generating a second set of subject predispositions that incorporates the change; and
displaying, on the application platform, a plan that includes the second set of subject predispositions.

17. The user computing device of claim 16, wherein the plan comprises a list of recommended actions the subject should take for each of the second set of subject predispositions to achieve the target goal.

18. The user computing device of claim 16, further comprising:
determining, based on the target goal and/or the data associated with the subject, a coaching individual; and
automatically matching, based on the determined coaching individual, the coaching individual with the user.

19. The user computing device of claim 18, further comprising:
identifying interaction data between the coaching individual and the subject;
analyzing, using the processor, the interaction data with respect to a progress rate of the user toward the target goal;
determining, using the processor and based on the analyzed interaction data, an aspect of the interaction data that accelerates or impedes the progress rate of the subject toward the target goal; and
transmitting, by the user computing device, instructions to another computing device associated with the coaching individual to display the aspect.

20. A non-transitory computer-readable medium storing instructions executable by one or more computer processors of a computer system, the instructions when executed by the one or more computer processors cause the one or more computer processors to perform operations comprising:
detecting, on a graphical user interface of an application platform of a user computing device, a selection of a subject by a user;
accessing, based on the detecting, data associated with the subject, wherein the data comprises the subject microbiome data;
generating, by a processor, an overview report comprising a first set of subject predispositions; and
displaying, on the application platform, the generated overview report.
